(19) United States
(12) Patent Application Publication
(10) Pub. No.: US 2025/0049713 A1
(43) Pub. Date: Feb. 13, 2025

(54) LIPID NANOPARTICLES FOR DELIVERY OF AGENTS

(71) Applicants: Massachusetts Institute of Technology, Cambridge, MA (US); FUJIFILM Corporation, Tokyo (JP)

(72) Inventors: Sho Toyonaga, Cambridge, MA (US); Daniel G. Anderson, Framingham, MA (US); Theresa Raimondo, Greenville, RI (US); Dennis Zheng Shi, Somerville, MA (US)

(21) Appl. No.: 18/634,595

(22) Filed: Apr. 12, 2024

Related U.S. Application Data
(60) Provisional application No. 63/495,958, filed on Apr. 13, 2023.

(51) Int. Cl. A61K 9/127; A61K 9/51; A61K 31/7088

(52) U.S. Cl. CPC A61K 9/1272; A61K 9/5123; A61K 31/7088

(57) ABSTRACT

The present disclosure provides compositions which enable the delivery of cargo, including therapeutic agents such as RNA, to organs or tissues in addition to the liver, lungs, and spleen, such as brain, heart, kidney, and muscle tissue. More specifically, the compositions comprise a plurality of lipid particles comprising an agent, cationic lipids and ionizable lipid.

### RELATED APPLICATION

This application claims the benefit of U.S. Provisional Application No. 63/495,958, filed on Apr. 13, 2023. The entire teachings of the above application are incorporated herein by reference.

### INCORPORATION BY REFERENCE OF SEQUENCE LISTING

The content of the electronically submitted sequence listing, file name: Q300376 Sequence listing as filed; size: 6,831 bytes; and date of creation: Oct. 9, 2024, filed herewith, is incorporated herein by reference in its entirety.

### BACKGROUND OF THE INVENTION

Nucleic acid-based therapeutics are an emerging and promising drug class with the potential for broad therapeutic approaches for many currently untreatable diseases. Lipid nanoparticles (LNPs) are self-assembling nanostructures which encapsulate and deliver nucleic acids. A liver-targeting LNP encapsulating therapeutic small interfering RNA (siRNA) has been clinically approved for the treatment of patients with transthyretin-mediated amyloidosis (ONPATTRO™). Incorporating cationic lipids into liver-targeting LNPs has been demonstrated to have the potential to redirect LNPs to lung and spleen. However, despite significant efforts to expand the scope of intravenously administered LNPs, delivery of LNPs to organs other than the liver, lung and spleen has remained a challenge.

Accordingly, there remains a need for compositions which enable delivery of agents to organs other than or in addition to liver, lung and/or spleen.

### SUMMARY OF THE INVENTION

The present disclosure is based, at least in part, on the discovery of compositions which enable delivery of agents, particularly therapeutic agents, to organs or tissues in addition to the liver, lung, and/or spleen.

Some embodiments provide a composition comprising a plurality of lipid nanoparticles, wherein the lipid nanoparticles comprise an agent, an ionizable lipid and a fully saturated cationic lipid, wherein said lipid nanoparticle does not contain a zwitterionic phospholipid.

Some embodiments provide a composition comprising a plurality of lipid nanoparticles, wherein the lipid nanoparticles comprise an agent, an ionizable lipid, and a cationic lipid, wherein the cationic lipid is a lipid according to formula VI:

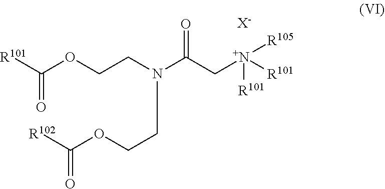

wherein values for the variables (e.g., R¹⁰¹, R¹⁰², R¹⁰⁵, X) are as defined herein.

Some embodiments provide a composition comprising a plurality of lipid nanoparticles, wherein the lipid nanoparticles comprise an agent, an ionizable lipid, and a cationic lipid, wherein the ionizable lipid is a lipid according to formula I or a pharmaceutically acceptable salt thereof:

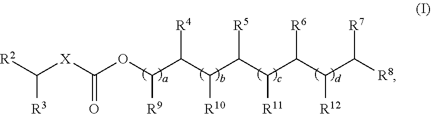

wherein values for the variables (e.g., X, R², R³, R⁴, R⁵, R⁶, R⁷, R⁸, R⁹, R¹⁰, R¹¹, R¹², a, b, c, d) are as defined herein.

Some embodiments provide a composition comprising a plurality of lipid nanoparticles, wherein the lipid nanoparticles comprise an agent, an ionizable lipid, and at least one of EDPPC and DPTAP, wherein the ionizable lipid is a lipid according to formula I or II or a pharmaceutically acceptable salt thereof:

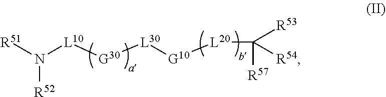

wherein values for the variables (e.g., R⁵¹, R⁵², L¹⁰, G³⁰, a′, L³⁰, G¹⁰, L²⁰, b′, R⁵³, R⁵⁴, R⁵⁷) are as defined herein.

The present disclosure additionally provides, in some embodiments, a method of delivering an agent to one or more of the brain, heart, muscles, and kidneys of a subject in need thereof, comprising systemically administering to the subject a composition comprising a plurality of lipid nanoparticles, wherein the lipid nanoparticles comprise the agent, an ionizable lipid, and a fully saturated cationic lipid.

Some embodiments provide a method of delivering an agent to a subject in need thereof, comprising administering to the subject a composition of the present disclosure.

Some embodiments provide a method of treating a subject having a disease, disorder or condition beneficially treated by an agent, comprising administering to the subject a therapeutically effective amount of a composition of the present disclosure comprising the agent.

Also provided herein, in some embodiments, is use of a composition of the present disclosure in accordance with a method described herein, e.g., use of a composition of the present disclosure comprising an agent for delivering the agent to a subject (e.g., a subject in need thereof); use of a composition of the present disclosure comprising an agent for treating a subject having a disease, disorder or condition beneficially treated by the agent. Also provided herein, in some embodiments, is use of a composition of the present disclosure in the manufacture of a medicament for use in a method described herein e.g., delivering an agent to a subject (e.g., a subject in need thereof); treating a subject having a disease, disorder or condition beneficially treated by an agent. Also provided herein, in some embodiments, is a composition for use in accordance with a method described herein, e.g., delivering an agent to a subject (e.g., a subject in need thereof); treating a disease, disorder or condition beneficially treated by an agent.

Surprisingly, it has been discovered that by incorporating certain cationic lipids into liver hepatocyte-targeting LNPs at optimized molar percentages, the LNPs can deliver RNA to various organs in addition to liver, lung, and spleen, including brain and muscle endothelial cells. In addition, by adjusting the cationic lipid ratio in LNPs, RNAs have been successfully delivered to organs in addition to the liver and lung. Vigorous formulation screening identified the cationic lipids 1,2-dipalmitoyl-sn-glycero-O-ethyl-3-phosphocholine (EDPPC), 1,2-dipalmitoyl-3-trimethylammonium-propane (DPTAP) and O,O′-ditetradecanoyl-N-(α-trimethylammonioacetyl) diethanolamine (DC-6-14), which enable RNA delivery to endothelial cells much more efficiently than 1,2-dioleoyl-3-trimethylammonium-propane (DOTAP) and 1,2-dimyristoyl-sn-glycero-O-ethyl-3-phosphocholine (EDMPC).

### BRIEF DESCRIPTION OF THE FIGURES

FIG. 1 shows the name and structure of several ionizable lipids and classes of ionizable lipids that may be suitable for use in the present disclosure.

FIG. 2A-2G shows the results of replacing a zwitterionic phospholipid with a cationic lipid. In this experiment LNPs encapsulating siRNA against VE-cadherin (Cdh5) were intravenously administered to mice at 0.5 mg/kg and organs were harvested 72 hours after injection. After isolation and purification of total RNA from organs, VE-cadherin (Cdh5) mRNA was quantified relative to a housekeeping gene, Gusb using RT-qPCR. FIG. 2A shows the data from this experiment in the liver. FIG. 2B shows the data from this experiment in the spleen. FIG. 2C shows the data from this experiment in the kidneys. FIG. 2D shows the data from this experiment in the lungs. FIG. 2E shows the data from this experiment in the heart. FIG. 2F shows the data from this experiment in the brain. FIG. 2G shows the data from this experiment in muscle.

FIG. 3A-3E shows a comparison of 20% EDOPC versus (vs.) 50% DOTAP in an LNP for delivery to selected organs. LNPs encapsulating siRNA against VE-cadherin (Cdh5) were intravenously administered to mice at 0.3 mg/kg and organs were harvested 72 hours after injection. After isolation and purification of total RNA from organs, VE-cadherin (Cdh5) mRNA was quantified relative to a housekeeping gene, Gusb using RT-qPCR. FIG. 3A shows the data from this experiment in the liver. FIG. 3B shows the data from this experiment in the spleen. FIG. 3C shows the data from this experiment in the kidneys. FIG. 3D shows the data from this experiment in the lungs. FIG. 3E shows the data from this experiment in the heart.

FIG. 4A-4G shows the results of a screen of cationic lipids at 20 mol % in LNPs in selected organs. Formulations including an siRNA against VE-cadherin were intravenously administered to mice at 0.5 mg/kg and organs were harvested 72 hours after injection. After isolation and purification of total RNA from organs, VE-cadherin (Cdh5) mRNA was quantified relative to a housekeeping gene, Gusb. A PBS control was also tested. FIG. 4A shows the data from this experiment in the liver. FIG. 4B shows the data from this experiment in the spleen. FIG. 4C shows the data from this experiment in the kidneys. FIG. 4D shows the data from this experiment in the lungs. FIG. 4E shows the data from this experiment in the heart. FIG. 4F shows the data from this experiment in the brain. FIG. 4G shows the data from this experiment in muscle.

FIG. 5A-5G shows the comparison of EDPPC and EDMPC at 20 mol % in LNPs with MC3 and MD1 as ionizable lipid. LNPs encapsulating siRNA against Cdh5 were intravenously administered to mice at 0.5 mg/kg and organs were harvested 72 hours after injection. After isolation and purification of total RNA from the organs, Cdh5 mRNA was quantified relative to a housekeeping gene, Gusb. FIG. 5A shows the data from this experiment in the liver. FIG. 5B shows the data from this experiment in the spleen. FIG. 5C shows the data from this experiment in the kidneys. FIG. 5D shows the data from this experiment in the lungs. FIG. 5E shows the data from this experiment in the heart. FIG. 5F shows the data from this experiment in the brain. FIG. 5G shows the data from this experiment in muscle.

FIG. 6A-6G shows the comparison of DPTAP and DOTAP at 20 mol % in LNPs with MC3 and MD1 as ionizable lipid. LNPs encapsulating siRNA against Cdh5 were intravenously administered to mice at 0.5 mg/kg and organs were harvested 72 hours after injection. After isolation and purification of total RNA from the organs, Cdh5 mRNA was quantified relative to a housekeeping gene, Gusb. FIG. 6A shows the data from this experiment in the liver. FIG. 6B shows the data from this experiment in the spleen. FIG. 6C shows the data from this experiment in the kidneys. FIG. 6D shows the data from this experiment in the lungs. FIG. 6E shows the data from this experiment in the heart. FIG. 6F shows the data from this experiment in the brain. FIG. 6G shows the data from this experiment in muscle.

FIG. 7A-7D shows the improvement in RNA delivery when an ionizable lipid and cationic lipid are used in combination compared to separately. LNPs encapsulating siRNA against Cdh5 were intravenously administered to mice at 0.3 mg/kg and organs were harvested 72 hours after injection. After isolation and purification of total RNA from the organs, Cdh5 mRNA was quantified relative to a housekeeping gene, Gusb. FIG. 7A shows the data from this experiment in the liver. FIG. 7B shows the data from this experiment in the lungs. FIG. 7C shows the data from this experiment in the heart. FIG. 7D shows the data from this experiment in the muscle.

FIG. 8A-8G shows the results of 20 mol % EDPPC in combination with various ionizable lipids. Formulations encapsulating an siRNA against Cdh5 were intravenously administered to mice at 0.5 mg/kg and organs were harvested 72 hours after injection. After isolation and purification of total RNA from organs, VE-cadherin mRNA was quantified relative to a housekeeping gene, GusB. A PBS control was also tested. FIG. 8A shows the data from this experiment in the liver. FIG. 8B shows the data from this experiment in the spleen. FIG. 8C shows the data from this experiment in the kidneys. FIG. 8D shows the data from this experiment in the lungs. FIG. 8E shows the data from this experiment in the heart. FIG. 8F shows the data from this experiment in the brain. FIG. 8G shows the data from this experiment in muscle.

FIG. 9A-9G shows the results of 20 mol % EDPPC in combination with various ionizable lipids. Formulations encapsulating an siRNA against Cdh5 were intravenously administered to mice at 0.5 mg/kg and organs were harvested 72 hours after injection. After isolation and purification of total RNA from organs, VE-cadherin mRNA was quantified relative to a housekeeping gene, GusB. A PBS control was also tested. FIG. 9A shows the data from this experiment in the liver. FIG. 9B shows the data from this experiment in the spleen. FIG. 9C shows the data from this experiment in the kidneys. FIG. 9D shows the data from this experiment in the lungs. FIG. 9E shows the data from this experiment in the heart. FIG. 9F shows the data from this experiment in the brain. FIG. 9G shows the data from this experiment in muscle.

FIG. 10A-10G shows the results of 20 mol % EDPPC in combination with various ionizable lipids. Formulations encapsulating an siRNA against Cdh5 were intravenously administered to mice at 0.5 mg/kg and organs were harvested 72 hours after injection. After isolation and purification of total RNA from organs, VE-cadherin mRNA is quantified relative to a housekeeping gene, GusB. A PBS control was also tested. FIG. 10A shows the data from this experiment in the liver. FIG. 10B shows the data from this experiment in the spleen. FIG. 10C shows the data from this experiment in the kidneys. FIG. 10D shows the data from this experiment in the lungs. FIG. 10E shows the data from this experiment in the heart. FIG. 10F shows the data from this experiment in the brain. FIG. 10G shows the data from this experiment in muscle.

FIG. 11A-11G shows the results of 20 mol % DPTAP in combination with various ionizable lipids. Formulations including an siRNA against VE-cadherin were intravenously administered to mice at 0.5 mg/kg and organs were harvested 72 hours after injection. After isolation and purification of total RNA from organs, VE-cadherin mRNA was quantified relative to a housekeeping gene, GusB. A PBS control was also tested. FIG. 11A shows the data from this experiment in the liver. FIG. 11B shows the data from this experiment in the spleen. FIG. 11C shows the data from this experiment in the kidneys. FIG. 11D shows the data from this experiment in the lungs. FIG. 11E shows the data from this experiment in the heart. FIG. 11F shows the data from this experiment in the brain. FIG. 11G shows the data from this experiment in muscle.

FIG. 12A-12G shows the results of 20 mol % DPTAP in combination with various ionizable lipids. Formulations including an siRNA against VE-cadherin were intravenously administered to mice at 0.5 mg/kg and organs were harvested 72 hours after injection. After isolation and purification of total RNA from organs, VE-cadherin mRNA was quantified relative to a housekeeping gene, GusB. A PBS control was also tested. FIG. 12A shows the data from this experiment in the liver. FIG. 12B shows the data from this experiment in the spleen. FIG. 12C shows the data from this experiment in the kidneys. FIG. 12D shows the data from this experiment in the lungs. FIG. 12E shows the data from this experiment in the heart. FIG. 12F shows the data from this experiment in the brain. FIG. 12G shows the data from this experiment in muscle.

FIG. 13A-13G shows the results of 20 mol % DC-6-14 in combination with various ionizable lipids. Formulations including an siRNA against VE-cadherin were intravenously administered to mice at 0.5 mg/kg and organs were harvested 72 hours after injection. After isolation and purification of total RNA from organs, VE-cadherin mRNA was quantified relative to a housekeeping gene, GusB. A PBS control was also tested. FIG. 13A shows the data from this experiment in the liver. FIG. 13B shows the data from this experiment in the spleen. FIG. 13C shows the data from this experiment in the kidneys. FIG. 13D shows the data from this experiment in the lungs. FIG. 13E shows the data from this experiment in the heart. FIG. 13F shows the data from this experiment in the brain. FIG. 13G shows the data from this experiment in muscle.

FIG. 14A-14G shows the results of 20 mol % DC-6-14 in combination with various ionizable lipids. Formulations including an siRNA against VE-cadherin were intravenously administered to mice at 0.5 mg/kg and organs were harvested 72 hours after injection. After isolation and purification of total RNA from organs, VE-cadherin mRNA was quantified relative to a housekeeping gene, GusB. A PBS control was also tested. FIG. 14A shows the data from this experiment in the liver. FIG. 14B shows the data from this experiment in the spleen. FIG. 14C shows the data from this experiment in the kidneys. FIG. 14D shows the data from this experiment in the lungs. FIG. 14E shows the data from this experiment in the heart. FIG. 14F shows the data from this experiment in the brain. FIG. 14G shows the data from this experiment in muscle.

FIG. 15A-15G shows the results of experiments to optimize the ratio of EDPPC in the LNP. To optimize the cationic lipid ratio for extrahepatic delivery, a series of LNPs with systematically increasing percentage of EDPPC were prepared and tested in vivo with single 0.5 mg/kg siCdh5 administration. LNPs containing 10-50% EDDPC showed efficacious Cdh5 gene silencing in liver, spleen, kidney, lung, and brain. LNP containing 20-30% EDPPC showed most potent gene silencing in heart and skeletal muscle. A PBS control was also tested. FIG. 15A shows the data from this experiment in the liver. FIG. 15B shows the data from this experiment in the spleen. FIG. 15C shows the data from this experiment in the kidneys. FIG. 15D shows the data from this experiment in the lungs. FIG. 15E shows the data from this experiment in the heart. FIG. 15F shows the data from this experiment in the brain. FIG. 15G shows the data from this experiment in muscle.

FIG. 16A-16G shows the results of experiments to optimize the ratio of DPTAP in the LNP. To optimize the cationic lipid ratio for extrahepatic delivery, a series of LNPs with systematically increasing percentage of DPTAP were prepared and tested in vivo with single 0.5 mg/kg siCdh5 administration. LNPs containing 10-50% DPTAP showed efficacious Cdh5 gene silencing in liver, spleen, kidney, lung, heart, and skeletal muscle A PBS control was also tested. FIG. 16A shows the data from this experiment in the liver. FIG. 16B shows the data from this experiment in the spleen. FIG. 16C shows the data from this experiment in the kidneys. FIG. 16D shows the data from this experiment in the lungs. FIG. 16E shows the data from this experiment in the heart. FIG. 16F shows the data from this experiment in the brain. FIG. 16G shows the data from this experiment in muscle.

FIG. 17A-17G shows the results of experiments to optimize the ratio of DC-6-14 in the LNP. To optimize the cationic lipid ratio for extrahepatic delivery, a series of LNPs with systematically increasing percentage of DC-6-14 were prepared and tested in vivo with single 0.5 mg/kg siCdh5 administration. LNPs containing 10-50% DC-6-14 showed efficacious Cdh5 gene silencing in liver, spleen, kidney, lung, heart, and skeletal muscle A PBS control was also tested. FIG. 17A shows the data from this experiment in the liver. FIG. 17B shows the data from this experiment in the spleen. FIG. 17C shows the data from this experiment in the kidneys. FIG. 17D shows the data from this experiment in the lungs. FIG. 17E shows the data from this experiment in the heart. FIG. 17F shows the data from this experiment in the brain. FIG. 17G shows the data from this experiment in muscle.

FIG. 18 shows the efficacy of siRNA delivery to the lungs when using a liver-targeting ionizable lipid in combination with DOTAP. To examine whether 50% DOTAP incorporation into liver-targeting LNPs comprising ionizable lipids of formula I or III redirects to the lung, five LNPs were prepared with different ionizable lipid (MC3 as a positive control), including one LNP without ionizable lipid as a negative control. Their lung endothelial delivery efficiency in vivo was tested using a single 0.4 mg/kg siCdh5 intravenous administration. The data from this experiment are shown.

FIG. 19A-19E shows the results on tissue tropism for changing the alkyl tail length of the PEG lipid in the LNP. To examine whether alkyl chain length of PEG-lipid affects tissue tropism of LNPs containing 50% DOTAP, three LNPs with different PEG lipids were prepared and tested for their endothelial delivery efficiency in vivo using a single 1.0 mg/kg siCdh5 intravenous administration. Three days post administration, the tissues were harvested and Cdh5 mRNA was quantified by RT-qPCR. All the LNPs showed comparable Cdh5 gene silencing in all the tissues collected. FIG. 19A shows the data from this experiment in the liver. FIG. 19B shows the data from this experiment in the spleen. FIG. 19C shows the data from this experiment in the kidneys. FIG. 19D shows the data from this experiment in the lungs. FIG. 19E shows the data from this experiment in the heart.

FIG. 20A-20G shows the results of 20 mol % EDOPC in combination with various ionizable lipids. To examine the generalizability of EDOPC for extrahepatic delivery, various ionizable lipids were formulated with 20% EDOPC. LNPs with five different ionizable lipids were prepared, and their endothelial delivery efficiency tested in vivo using a single 0.5 mg/kg siCdh5 intravenous administration. Three days post administration, the tissues were harvested and Cdh5 mRNA was quantified by RT-qPCR. All the LNPs containing 20% EDOPC showed significant Cdh5 gene silencing in all the tissues tested, indicating that these novel formulations can be used for RNA delivery to the endothelial RNA delivery in various tissues. FIG. 20A shows the data from this experiment in the liver. FIG. 20B shows the data from this experiment in the spleen. FIG. 20C shows the data from this experiment in the kidneys. FIG. 20D shows the data from this experiment in the lungs. FIG. 20E shows the data from this experiment in the heart. FIG. 20F shows the data from this experiment in the brain. FIG. 20G shows the data from this experiment in muscle.

FIG. 21A-21E shows the results on tissue tropism for changing the alkyl tail length of the PEG lipid in an LNP with 18.5% EDOPC. To examine whether alkyl chain length of the PEG-lipid affects tissue tropism of LNPs containing 18.5% EDOPC, three LNPs with different PEG lipids were prepared and tested for their endothelial delivery efficiency in vivo using a single 1.0 mg/kg siCdh5 intravenous administration. Three days post administration, the tissues were harvested and Cdh5 mRNA was quantified by RT-qPCR. All the LNPs showed comparable Cdh5 gene silencing in all the tissues collected. FIG. 21A shows the data from this experiment in the liver. FIG. 21B shows the data from this experiment in the spleen. FIG. 21C shows the data from this experiment in the kidneys. FIG. 21D shows the data from this experiment in the lungs. FIG. 21E shows the data from this experiment in the heart.

FIG. 22A-22F shows the results of 10 mol % DPTAP in combination with various ionizable lipids. To examine the generalizability of DPTAP for extrahepatic delivery, various ionizable lipids were formulated with 10% DPTAP. LNPs with six different ionizable lipids were prepared, and their endothelial delivery efficiency tested in vivo using a single 0.5 mg/kg siCdh5 intravenous administration. Three days post administration, the tissues were harvested and Cdh5 mRNA was quantified by RT-qPCR. All the LNPs containing 10% DPTAP showed significant Cdh5 gene silencing in all the tissues tested, indicating that these novel formulations can be used for RNA delivery to the endothelial RNA delivery in various tissues. FIG. 22A shows the data from this experiment in the liver. FIG. 22B shows the data from this experiment in the kidneys. FIG. 22C shows the data from this experiment in the lungs. FIG. 22D shows the data from this experiment in the heart. FIG. 22E shows the data from this experiment in the brain. FIG. 22F shows the data from this experiment in the muscle.

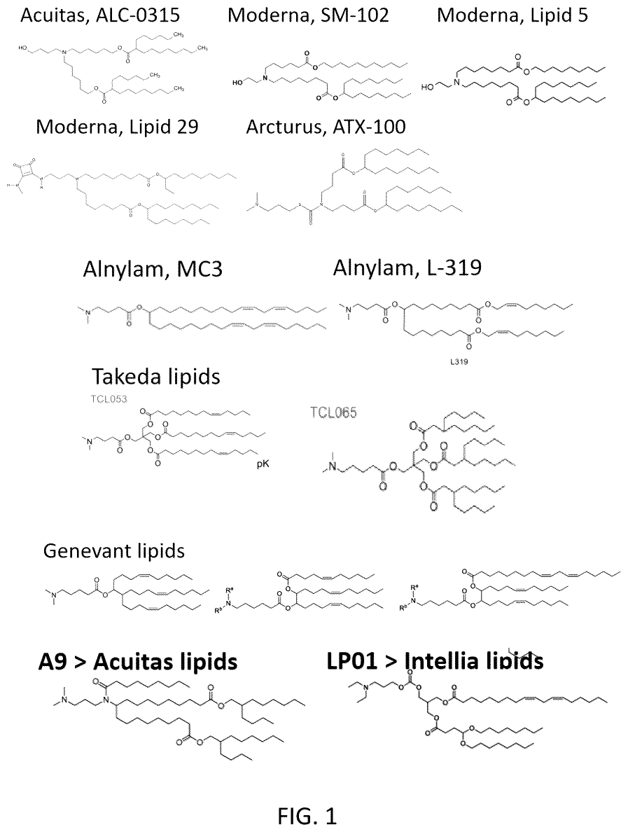

### DETAILED DESCRIPTION

Unless otherwise defined, all technical and scientific terms used herein have the same meaning as commonly understood by one of ordinary skill in the art to which this disclosure belongs. Although methods and materials similar or equivalent to those described herein can be used in accordance with the present disclosure, suitable methods and materials are described below. All publications, patent applications, patents, and other references mentioned herein are incorporated by reference in their entirety. In addition, the materials, methods, and examples are illustrative only and not intended to be limiting. Other features and advantages of the present disclosure will be apparent from the following detailed description, and from the claims.

### A. Definitions

The compounds described herein include any and all possible isomers, stereoisomers, enantiomers diastereomers, tautomers, salt (e.g., pharmaceutically acceptable salts), and solvates thereof. Thus, the terms “compound” and “compounds” as used in this disclosure refer to the compounds of this disclosure and any of all possible isomers, stereoisomers, enantiomers diastereomers, tautomers, pharmaceutically-acceptable salts, and solvates thereof. This applies except where otherwise specifically noted either in text or by use of standard notation of the art to depict particular regioisomers, stereoisomers, epimers, enantiomers, or configurations.

Compounds described herein may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers (including cis and trans double bond isomers), conformational isomers (including rotamers and atropisomers), tautomers and intermediate mixtures, with all possible isomers and mixtures thereof being included, unless otherwise indicated.

When a compound is depicted by structure without indicating the stereochemistry, and the compound has one or more chiral centers, it is to be understood that the structure encompasses one enantiomer or diastereomer of the compound separated or substantially separated from the corresponding optical isomer(s), a racemic mixture of the compound, and mixtures enriched in one enantiomer or diastereomer relative to its corresponding optical isomer(s). When a compound is depicted by a structure indicating stereochemistry, and the compound has more than one chiral center, the stereochemistry indicates relative stereochemistry, rather than the absolute configuration of the substituents around the one or more chiral carbon atoms. Unless indicated otherwise at the point of use herein, when a compound is depicted by a structure indicating stereochemistry using solid and/or broken wedges, and the compound has one or more chiral centers, the stereochemistry indicates absolute configuration of the substituents around the one or more chiral centers. Unless indicated otherwise as the point of use herein, when a compound is depicted by a structure indicating stereochemistry using solid and/or broken bolded lines, and the compound has one or more chiral centers, the stereochemistry indicates relative stereochemistry of unspecified absolute configuration of the substituents around the one or more chiral centers. “R” and “S” can also or alternatively be used to indicate the absolute configuration of substituents around one or more chiral centers (e.g. carbon atoms).

Thus, for example, a single stereoisomer with known relative and absolute configuration of two chiral centers can be designated using the conventional RS system (e.g., (1S,2S)); diastereomers in a racemic mixture can be designated using the RS system with two letters (e.g., (1RS,2RS) as a racemic mixture of (1R,2R) and (1S,2S); (1RS,2SR) as a racemic mixture of (1R,2S) and (1S,2R)).

The articles “a” and “an” are used in this disclosure to refer to one or more than one (i.e., to at least one) of the grammatical object of the article. By way of example, “an element” means one element or more than one element. Likewise, when introducing elements disclosed herein, the articles “a,” “an,” “the,” and “said” are intended to mean that there are one or more of the elements. Further, the one or more elements may be the same or different.

The term “or” is used in this disclosure to mean, and is used interchangeably with, the term “and/or,” unless indicated otherwise.

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, e.g., ±10%, ±5% or ±1% of a given value. It is to be understood that the term “about” can precede any particular value specified herein, except for particular values used in the Exemplification.

Unless otherwise specifically defined, “alkyl” refers to a linear or branched hydrocarbon radical, which is fully saturated and can include divalent radicals, having from 1 to about 30 carbon atoms if it is saturated, or from 2 to about 30 carbon atoms if it is unsaturated. Examples of saturated alkyl include, but are not limited to, groups such as methyl (Me), ethyl (Et), n-propyl, isopropyl, n-butyl, t-butyl, isobutyl, sec-butyl, isomers of, for example, n-pentyl, n-hexyl, n-heptyl, n-octyl, 1,1-dimethyl-heptyl, 1,2-dimethyl-heptyl, and the like. An unsaturated alkyl may include one or more double bonds, triple bonds or combinations thereof. Examples of unsaturated alkyl include, but are not limited to, vinyl, propenyl, isopropenyl, crotyl, 2-isopentenyl, allenyl, butenyl, butadienyl, pentenyl, pentadienyl, 3-(1,4-pentadienyl), hexenyl, hexadienyl, ethynyl, propynyl, butynyl, and other isomers. The term “divalent alkyl radicals” unless otherwise specifically defined refers to the general formula:-alkyl-. Divalent alkyl radicals refer to alkyl groups with two points of attachment to a larger structure and/or other substituents. The term “C₁₋ₙ-alkyl” refers to an alkyl having from 1 to about n carbon atoms, where n is an integer greater than 1.

“Alkenyl” refers to a branched-or straight-chain, aliphatic radical having the specified number of carbon atoms and at least one (e.g., one, two, three, four, five, etc.) carbon-carbon double bonds. Thus, “(C₁-C₁₅) alkenyl” refers to a radical having from 1-15 carbon atoms and at least one carbon-carbon double bond in a branched or linear arrangement. In some embodiments, alkenyl is (C₁-C₃₀) alkenyl, e.g., (C₁-C₂₀) alkenyl, (C₁-C₁₅) alkenyl, (C₁-C₁₀) alkenyl, (C₁-C₅) alkenyl or (C₁-C₃) alkenyl. Examples of alkyl groups include vinyl, allyl, and the like. In some embodiments, alkenyl is optionally substituted, e.g., with one or more substituents described herein.

The term “alkynyl” refers to a brached- or straight-chain aliphatic group with a carbon atom as the point of attachment and at least one carbon-carbon triple bond. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

As used herein, the term “hydrocarbon” refers to aliphatic moieties comprised of carbon and hydrogen. A “hydrocarbon group” is considered to be an alkyl, alkenyl, or alkynl group, or a combination thereof.

“Alkoxy” refers to an alkyl radical attached through an oxygen linking atom, wherein alkyl is as described herein. Examples of alkoxy include, but are not limited to, methoxy, ethoxy, propoxy, isopropoxy, and the like.

“Aryl” refers to a monocyclic or polycyclic (e.g., bicyclic, tricyclic), carbocyclic, aromatic ring system having the specified number of ring atoms, and includes aromatic ring(s) fused to non-aromatic rings, as long as one of the fused rings is an aromatic ring comprised of hydrogen and carbon. Thus, “(C₆-C₁₅) aryl” means an aromatic ring system having from 6-15 ring atoms. In some embodiments, aryl is (C₆-C₂₅) aryl, for example, (C₆-C₂₀) aryl, (C₆-C₁₅) aryl, (C₆-C₁₂) aryl or (C₆-C₁₀) aryl. Examples of aryl include phenyl, naphthyl and fluorenyl. In some embodiments, aryl is phenyl or fluorenyl.

The term “heteroaryl” refers to a monovalent aromatic group with an aromatic carbon atom or nitrogen atom as the point of attachment, the carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen, and aromatic sulfur. Heteroaryl rings may contain 1, 2, 3, or 4 ring atoms selected from nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl, isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “heteroaryl” may also refer to a divalent aromatic group, with two aromatic carbon atoms, two aromatic nitrogen atoms, or one aromatic carbon atom and one aromatic nitrogen atom as the two points of attachment, the atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkyl, or alkenyl groups (carbon number limitation permitting). As used herein, the term does not preclude the presence of one or more alkyl, aryl, and/or aralkyl groups (carbon number limitation permitting) attached to the aromatic ring or aromatic ring system.

Unless otherwise specifically defined, the term “cycloalkyl” refers to a fully saturated cyclic alkyl group containing from 1 to 4 rings and 3 to 8 carbon atoms per ring. Exemplary such groups include, but are not limited to, cyclopropyl, cyclobutyl, cyclopentyl, cyclohexyl, cycloheptyl, adamantyl, bicyclo[1.1.1]pentane, etc.

In general, “substituted” or “optionally substituted” refers to groups (e.g., an alkyl group, an aryl group) in which one or more bonds to a hydrogen atom contained therein may be replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, which may be the same or different, unless otherwise specified. In some embodiments, a substituted group is substituted with 1, 2, 3, 4, 5, or 6 substituents. It will be understood that “substitution” or “substituted with” includes the implicit proviso that such substitution is in accordance with permitted valence of the substituted atom. Examples of substituent groups include, but are not limited to, alkyl, alkynyl, alkenyl, aryl, heteroaryl, aralkyl, alkaryl, alcohol, halogens, hydroxyls; alkoxy, alkenoxy, alkynoxy, aryloxy, aralkyloxy, heterocyclyloxy, heterocyclylalkoxy groups, carbonyls, carboxyls, esters, urethanes, oximes, hydroxylamines, alkoxyamines, aralkoxyamines, thiols, sulfides, sulfoxides, sulfones, sulfonyls, sulfonamides, sulfonates, sulfates, amines, N-oxides, hydrazines, hydrazides, hydrazones, azides, amides, ureas, amidines, guanidines, enamines, imides, isocyanates, isothiocyanates, cyanates, thiocyanates, imines, nitro groups, nitriles, ═CF₂, —CF₃, —CF₂H, ═CH₂, —CHO, C(halogen)₃, —ONO₂, -alkyl-ONO₂, —C(O)OP(O)(O-alkyl)₂, —OPO₃H₂, and polyethylene glycol.

“Hydroxy” refers to —OH.

Unless otherwise specifically defined, “alkylamino” refers to the general formula —(NH)-alkyl. Unless otherwise specifically defined, “di-alkylamino” refers to the general formula —N-(alkyl)₂. Unless otherwise specifically limited di-alkylamino includes cyclic amine compounds such as piperidine, piperazine, azetidine, pyrrolidine, morpholine and their derivatives.

The terms “salt” and “salts”, as employed in the disclosure, denotes acidic and/or basic salts formed with inorganic and/or organic acids and bases.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds and lipids described herein include pharmaceutically acceptable acid addition salts and pharmaceutically acceptable base addition salts. In some embodiments, one or more components of the pharmaceutically acceptable salt is not an acid addition salt or base addition salt. The term “pharmaceutically acceptable salt” is intended to include salts derived from inorganic or organic acids including, for example hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2 sulfonic and other acids. Pharmaceutically acceptable salt forms may also include forms wherein the ratio of molecules comprising the salt is not 1:1. For example, the salt may comprise more than one inorganic or organic acid molecule per molecule of base, such as two hydrochloric acid molecules per molecule of a formula provided herein. As another example, the salt may comprise less than one inorganic or organic acid molecule per molecule of base, such as two molecules of a lipid or a compound of a formula provided herein per molecule of tartaric acid. Additional examples of pharmaceutically acceptable acid addition salts are salts of an amino group formed with inorganic acids such as hydrochloric acid, hydrobromic acid, phosphoric acid, sulfuric acid and perchloric acid, or with organic acids, such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like. Either the mono-, di- or tri-acid salts can be formed, and such salts can exist in either a hydrated, solvated or substantially anhydrous form.

Pharmaceutically acceptable base addition salts include salts formed with inorganic bases, such as alkali metal, alkaline earth metal, and ammonium bases, and salts formed with aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable base addition salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease, a stabilized (i.e., not worsening) state of disease, preventing spread of disease, delay or slowing of disease progression, amelioration or palliation of the disease state and remission (whether partial or total), whether detectable or undetectable. “Treatment” can also mean prolonging survival as compared to expected survival if not receiving treatment.

In some embodiments, “treating,” as used herein, refers to taking steps to deliver a therapy to a subject, such as a mammal, in need thereof (e.g., as by administering to a subject one or more therapeutic agents). In some embodiments, “treating” also includes inhibiting a disease or condition (e.g., as by slowing or stopping its progression or causing regression of the disease or condition), and relieving the symptoms resulting from a disease or condition.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some aspects, a subject is a human.

As used herein an “effective amount” or “therapeutically effective amount” of a composition is the quantity of a composition which, when administered to a subject, results in a discernible physiological effect in the individual or animal. The compositions disclosed herein may have pharmacological properties when administered in therapeutically effective amounts for providing a physiological effect useful to treat a number of physiological conditions. The actual amount which comprises the “effective amount” or “therapeutically effective amount” will vary depending on a number of conditions including, but not limited to, the agent, the particular disorder being treated, the severity of the disorder, the size and health of the patient, and the route of administration. A skilled medical practitioner can readily determine the appropriate amount using methods known in the medical arts.

Additionally, “a therapeutically effective amount” can refer to an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual. A therapeutically effective amount of an agent to be administered can be determined by a clinician of ordinary skill using the guidance provided herein and other methods known in the art.

### B. Example Embodiments

Example embodiments of the present disclosure include:
1. A composition comprising a plurality of lipid nanoparticles, wherein the lipid nanoparticles comprise an agent, an ionizable lipid and a fully saturated cationic lipid, wherein said lipid nanoparticle does not contain a zwitterionic phospholipid.
2. The composition of embodiment 1, wherein the lipid nanoparticles have an apparent ionization constant (pKa) between about 4.5 and about 7.
3. The composition of embodiment 1 or 2, wherein the lipid nanoparticles have an apparent ionization constant (pKa) between about 5 and about 7.
4. The composition of any one of embodiments 1-3, wherein the lipid nanoparticles have an apparent ionization constant (pKa) between about 5.5 and about 7.
5. The composition of any one of embodiments 1-4, wherein the lipid nanoparticles have an apparent ionization constant (pKa) between about 6 and about 7.
6. The composition of any one of embodiments 1-5, wherein the cationic lipid is less than about 50 mol percent of the lipid nanoparticle.
7. The composition of any one of embodiments 1-6, wherein the cationic lipid is less than about 30 mol percent of the lipid nanoparticle.
8. The composition of any one of embodiments 1-7, wherein the cationic lipid is less than about 20 mol percent of the lipid nanoparticle.
9. The composition of any one of embodiments 1-8, wherein the fully saturated cationic lipid is a C₁₂-C₁₈ fully saturated cationic lipid.
10. The composition of any one of embodiments 1-9, further comprising a sterol.
11. The composition of any one of embodiments 1-10, further comprising a PEG-lipid.
12. The composition of any one of embodiments 1-11, wherein the agent is a bioactive agent.
13. The composition of embodiment 12, wherein the bioactive agent is a therapeutic agent.
14. The composition of embodiment 13, wherein the therapeutic agent is a nutraceutical agent.
15. The composition of any one of embodiments 1-11, wherein the agent is an imaging agent.
16. The composition of any one of embodiments 1-11, wherein the agent is a diagnostic agent.
17. The composition of any one of embodiments 1-11, wherein the agent is a nucleic acid.
18. The composition of embodiment 17, wherein the nucleic acid is a ribonucleic acid.
19. The composition of embodiment 17, wherein the nucleic acid is a deoxyribonucleic acid.
20. The composition of embodiment 17, wherein the nucleic acid is a non-natural nucleic acid.
21. The composition of any one of embodiments 1-20, wherein the ionizable lipid is a lipid according to formula I or II or a pharmaceutically acceptable salt thereof:

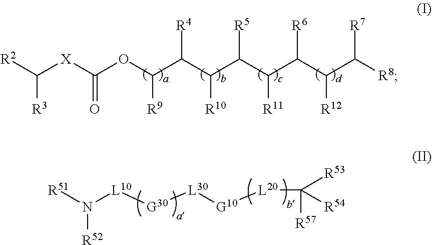

wherein
X is —NR¹— or —O—;
R¹ is a hydrogen atom, a hydrocarbon group having 6 to 24 atoms, or R²¹-L¹-R²²—;
R²¹ is a hydrocarbon group having 1 to 24 carbon atoms;
L¹ is —O(CO)O—, —O(CO), —(CO)O—, —O—, or

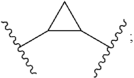 [SMILES: *C1CC1*]

R²² is a divalent hydrocarbon linking group having 1 to 18 carbon atoms;
R² and R³ are each independently a hydrogen atom, a hydrocarbon group having 3 to 24 carbon atoms, or R³¹-L¹-R³²—;
R³¹ is a hydrocarbon group having 1 to 24 carbon atoms;
L² is —O(CO)O—, —O(CO)—, —(CO)O—, —O—, or

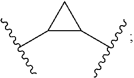 [SMILES: *C1CC1*]

R³² is a divalent hydrocarbon linking group having 1 to 18 carbon atoms;
R⁴, R⁵, R⁶, R⁷, R⁸, R⁹, R¹⁰, R¹¹, and R¹² are each independently a hydrogen atom or an alkyl group having 1 to 18 carbon atoms optionally substituted with R³³, where one or more pairs of groups selected from R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸ may be linked to each other to form a 4- to 7-membered ring of carbon and nitrogen atoms which may contain an oxygen atom;
R³³ is a hydroxyl group, a carboxyl group, —NR⁴⁵R⁴⁶, —O(CO)O—R⁴¹, —O(CO)—R⁴², —(CO)O—R⁴³, —O—R⁴⁴, or an aryl or heteroaryl group optionally substituted with R³⁴;
R³⁴ is an alkyl group having 1 to 18 carbon atoms, a hydroxyl group, a carboxyl group, —NR⁴⁵R⁴⁶, —O(CO)O—R⁴¹, —O(CO)—R⁴², —(CO)O—R⁴³, or —O—R⁴⁴;
R⁴¹, R⁴², R⁴³, R⁴⁴, R⁴⁵, and R⁴⁶ are each independently a hydrocarbon group having 1 to 18 carbon atoms;
a, b, c, and d are each independently an integer from 0 to 3, where a+b is 1 or more and c+d is 1 or more;
R⁵¹ and R⁵² are each independently an alkyl group having 1 to 18 carbon atoms optionally substituted with R³⁵;
R³⁵ is a hydroxyl group, -G²⁰-CH(R⁵⁵)(R⁵⁶), —N(R⁵⁸)(R⁵⁹), or -G²⁰-R⁶⁰.
G²⁰ is —(CO)O— or —O(CO)—;
R⁵⁵ and R⁵⁶ are each independently hydrogen or an alkyl group having 1 to 18 carbon atoms;
R⁵⁸ and R⁵⁹ are each independently hydrogen or a cycloalkyl group having 3 to 6 carbon atoms, optionally substituted with R³⁶;
R⁶⁰ is alkyl group having 1 to 18 carbon atoms or a cycloalkyl group having 3 to 6 carbon atoms;
R³⁶ is —N(R⁶¹)(R⁶²), or -G²⁰-R⁶⁵,
R⁶¹ and R⁶² are each independently hydrogen or a cycloalkyl group having 3 to 6 carbon atoms;
R⁶⁵ is an alkyl group having 1 to 18 carbon atoms, -L⁴⁰-CH(R⁶⁶)(R⁶⁷), or -G²⁰-R⁶⁶.
L⁴⁰ is a divalent alkyl group containing 1 to 6 carbon atoms;
R⁶⁶ and R⁶⁷ are each independently an alkyl group containing 1 to 10 carbon atoms or an alkoxy group containing 1 to 10 carbon atoms;
L¹⁰ is a divalent alkyl group containing 1 to 10 carbon atoms;
G³⁰ is —S(CO)N(R⁶⁴);
R⁶⁴ is -L³⁰-G²⁰-CH(R⁵⁵)(R⁵⁶);
a′ is 0 or 1;
G¹⁰ is G²⁰, —O(CO)O—, or —N(R⁶³)C(O)—;
R⁶³ is an alkyl group containing 1 to 18 carbon atoms;
L²⁰ is a divalent alkyl group containing 1 to 6 carbon atoms;
b′ is 0 or 1;
R⁵³, R⁵⁴, and R⁵⁷ are each independently hydrogen or an alkyl group containing 1 to 18 carbon atoms optionally substituted with R³⁶, and
L³⁰ is a single bond or an alkyl group containing 1 to 18 carbon atoms.
22. The composition of any one of embodiments 1-20, wherein the ionizable lipid is a lipid according to formula VII or a pharmaceutically acceptable salt thereof:

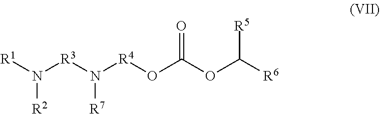

wherein
R¹ and R² are each independently a hydrocarbon group having 1 to 18 carbon atoms, and R³ is a hydrocarbon group having 2 to 8 carbon atoms, wherein the hydrocarbon groups represented by R¹, R², and R³ are each independently optionally substituted with one or more substituents selected from —OH, COOH, —NR⁵¹R⁵², —OC(O)O—R⁵³, —C(O)O—R⁵⁴, —OC(O)—R⁵⁵, and —O—R⁵⁶;
R⁴ is a hydrocarbon group having 1 to 8 carbon atoms;
R⁵ and R⁶ are each independently a hydrocarbon group having 1 to 8 carbon atoms or —R⁸-L¹-R⁹, excluding a case where both R⁵ and R⁶ are hydrocarbon groups having 1 to 8 carbon atoms;
R⁷ is —R¹⁰-L²-R¹¹-L³-R¹²,
R⁵¹ and R⁵² are each independently a hydrocarbon group having 1 to 8 carbon atoms;
R⁵³, R⁵⁴, R⁵⁵, and R⁵⁶ are each independently a hydrocarbon group having 1 to 24 carbon atoms;
the hydrocarbon groups represented by R⁵³, R⁵⁴, R⁵⁵, and R⁵⁶ are each independently optionally substituted with an aryl group having 6 to 20 carbon atoms or —S—R⁵⁸, wherein the aryl group having 6 to 20 carbon atoms is optionally substituted with —OH, COOH, —NR⁵¹R⁵², —OC(O)O—R⁵³, —C(O)O—R⁵⁴, —OC(O)—R⁵⁵, —O—R⁵⁶, or —R⁵⁹—R⁵⁷, wherein
R⁵⁸ and R⁵⁹ are each independently a hydrocarbon group having 1 to 12 carbon atoms;
R⁵⁷ is —OH, COOH, —NR⁶¹R⁶², —OC(O)O—R⁶³, —C(O)O—R⁶⁴, —OC(O)—R⁶⁵, or —O—R⁶⁶;
R⁶¹ and R⁶² are each independently a hydrocarbon group having 1 to 8 carbon atoms;
R⁶³, R⁶⁴, R⁶⁵, and R⁶⁶ are each independently a hydrocarbon group having 1 to 24 carbon atoms;
the hydrocarbon groups represented by R⁶³, R⁶⁴, R⁶⁵, and R⁶⁶ are each independently optionally substituted with an aryl group having 6 to 20 carbon atoms or —S—R⁶⁸ wherein the aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or —R⁶⁹—R⁶⁷,
R⁶⁸ and R⁶⁹ are each independently a hydrocarbon group having 1 to 12 carbon atoms;
R⁶⁷ is —OH, COOH, —NR⁶¹R⁶², —OC(O)O—R⁶³, —C(O)O—R⁶⁴, —OC(O)—R⁶⁵, or —O—R⁶⁶,
L¹, L², and L³ are each independently —OC(O)O—, —C(O)O—, —OC(O)—, or —O—;
R⁸ is a hydrocarbon group having 1 to 12 carbon atoms;
R⁹ is a hydrocarbon group having 1 to 24 carbon atoms;
R¹⁰ is a hydrocarbon group having 1 to 8 carbon atoms;
R¹¹ is a hydrocarbon group having 1 to 24 carbon atoms;
R¹² is a hydrocarbon group having 1 to 24 carbon atoms;
the hydrocarbon groups represented by R⁹ and R¹² are each independently optionally substituted with an aryl group, —OC(O)O—R⁵³, —C(O)O—R⁵⁴, —OC(O)—R⁵⁵, or —S—R⁵⁸; and
the hydrocarbon groups represented by R¹¹ are each independently optionally substituted with —OC(O)O—R⁵³, —C(O)O—R⁵⁴, or —OC(O)—R⁵⁵.
23. The composition of any one of embodiments 1-21, wherein the ionizable lipid is a lipid according to formula III or a pharmaceutically acceptable salt thereof:

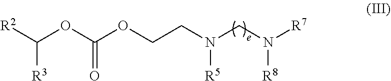

Wherein
R² and R³ are each independently a hydrogen atom, a hydrocarbon group having 3 to 24 carbon atoms, or R³¹-L²-R³²—;
R³¹ is a hydrocarbon group having 1 to 24 carbon atoms;
L² is —O(CO)O—, —O(CO), —(CO)O—, —O—, or

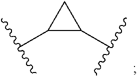 [SMILES: *C1CC1*]

R³² is a divalent hydrocarbon linking group having 1 to 18 carbon atoms;
R⁵ is a hydrogen atom or an alkyl group having 1 to 18 carbon atoms optionally substituted with R³³;
R⁷ and R⁸ are each independently a hydrogen atom or an alkyl group having 1 to 18 carbon atoms optionally substituted with R³³;
R³³ is a hydroxyl group, a carboxyl group, —NR⁴⁵R⁴⁶, —O(CO)O—R⁴¹, —O(CO)—R⁴², —(CO)O—R⁴³, —O—R⁴⁴, or an aryl or heteroaryl group optionally substituted with R³⁴;
R³⁴ is an alkyl group having 1 to 18 carbon atoms, a hydroxyl group, a carboxyl group, —NR⁴⁵R⁴⁶, —O(CO)O—R⁴¹, —O(CO)—R⁴², —(CO)O—R⁴³, or —O—R⁴⁴;
R⁴¹, R⁴², R⁴³, R⁴⁴, R⁴⁵, and R⁴⁶ are each independently a hydrocarbon group having 1 to 18 carbon atoms; and
e is an integer selected from 2 or 3.
24. The composition of any one of embodiments 1-23, wherein the ionizable lipid is selected from the group consisting of

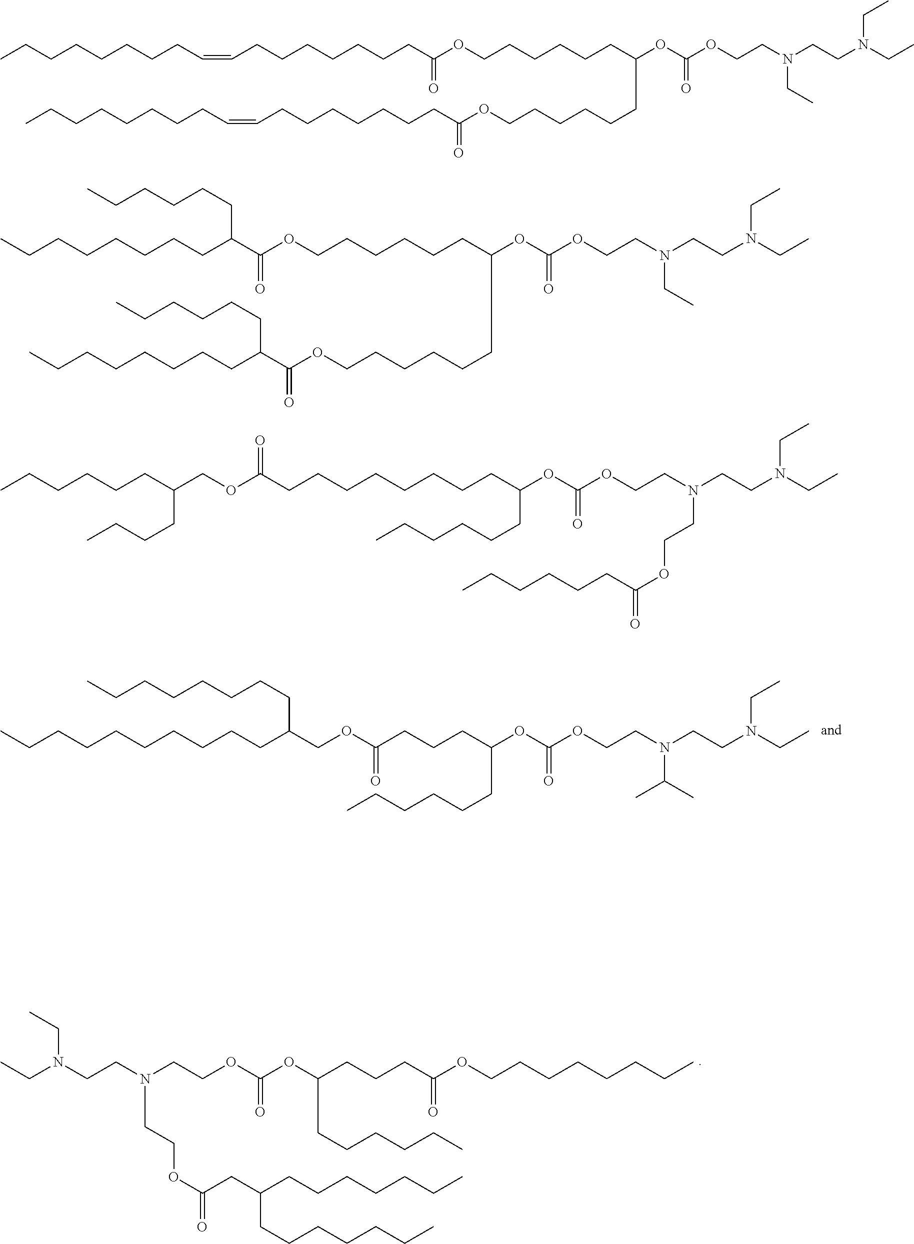

25. The composition of any one of embodiments 1-20 and 22, wherein the ionizable lipid is selected from the group consisting of

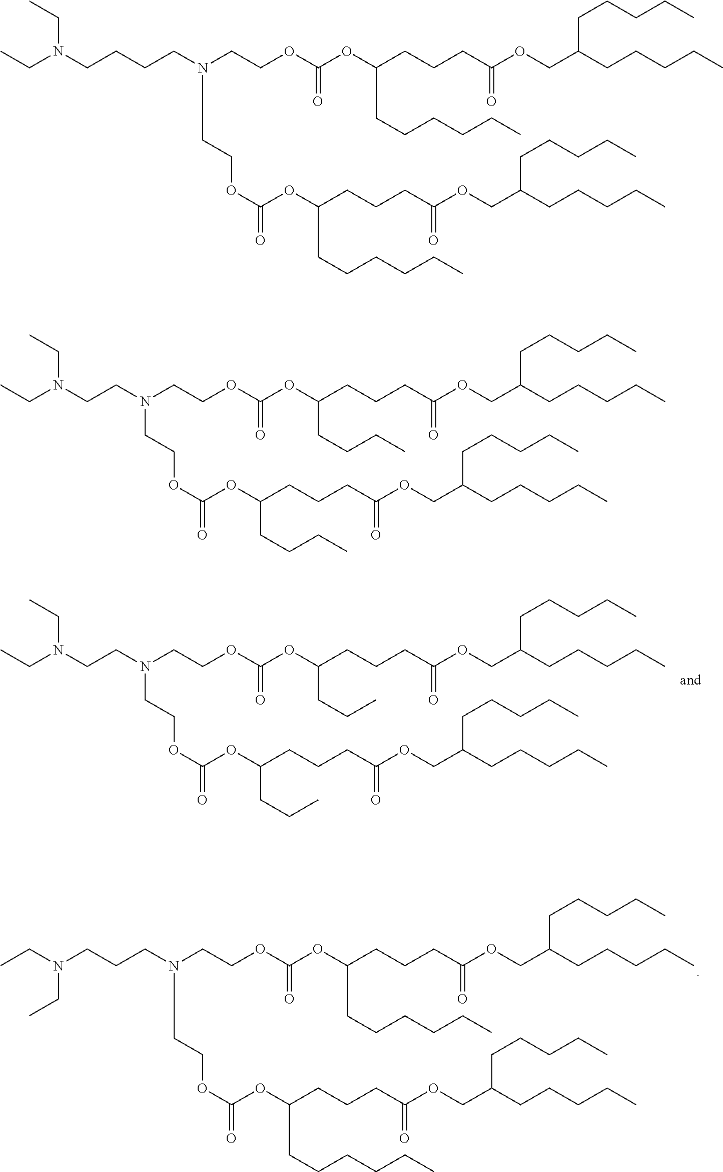

26. The composition of any one of embodiments 1-25, wherein the cationic lipid is a lipid according to formula IV:

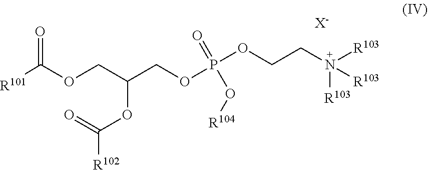

wherein:
R¹⁰¹ and R¹⁰² are each independently optionally substituted (C₈-C₂₄) alkyl or optionally substituted (C₈-C₂₄) alkenyl;
R¹⁰³ is independently in each instance optionally substituted (C₁-C₆) alkyl;
R¹⁰⁴ is optionally substituted (C₁-C₆) alkyl; and
X⁻ is a monovalent anion.
27. The composition of any one of embodiments 1-26, wherein the cationic lipid is 1,2-dilauroyl-sn-glycero-O-ethyl-3-phosphocholine (EDLPC), 1,2-dimyristoyl-sn-glycero-O-ethyl-3-phosphocholine (EDMPC), 1,2-dimyristoleoyl-sn-glycero-O-ethyl-3-phosphocholine (14:1 EPC), 1,2-dipalmitoyl-sn-glycero-O-ethyl-3-phosphocholine (EDPPC), 1,2-distearoyl-sn-glycero-O-ethyl-3-phosphocholine (EDSPC), 1,2-dioleoyl-sn-glycero-3-ethylphosphocholine (EDOPC), or 1-palmitoyl-2-oleoyl-sn-glycero-O-ethyl-3-phosphocholine (16:0-18:1 EPC).
28. The composition of any one of embodiments 1-27, wherein the cationic lipid is EDPPC.
29. The composition of any one of embodiments 1-25, wherein the cationic lipid is a lipid according to formula V:

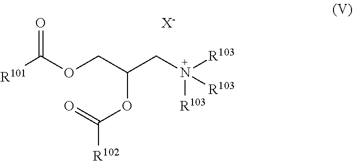

wherein:
R¹⁰¹ and R¹⁰² are each independently optionally substituted (C₈-C₂₄) alkyl or optionally substituted (C₈-C₂₄) alkenyl;
R¹⁰³ is independently in each instance optionally substituted (C₁-C₆) alkyl; and
X⁻ is a monovalent anion.
30. The composition of any one of embodiments 1-25 and 29, wherein the cationic lipid is selected from the group consisting of 1,2-dimyristoyl-3-trimethylammonium-propane (DMTAP), 1,2-dipalmitoyl-3-trimethylammonium-propane (DPTAP), 1,2-distearoyl-3-trimethylammonium-propane (DSTAP), 1,2-dioleoyl-3-trimethylammonium-propane (DOTAP), and N-(2-hydroxyethyl)-N,N-dimethyl-2,3-bis (oleoyloxy) propan-1-aminium (DORI).
31. The composition of any one of embodiments 1-25, 29, and 30, wherein the cationic lipid is DPTAP.
32. The composition of any one of embodiments 1-25, wherein the cationic lipid is a lipid according to formula VI:

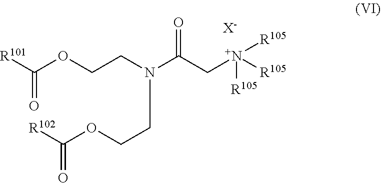

wherein:
R¹⁰¹ and R¹⁰² are each independently optionally substituted (C₈-C₂₄) alkyl or optionally substituted (C₈-C₂₄) alkenyl;
R¹⁰⁵ are each independently (C₁-C₆) alkyl; and
X⁻ is a monovalent anion.
33. The composition of any one of embodiments 1-25 and 32 wherein the cationic lipid is

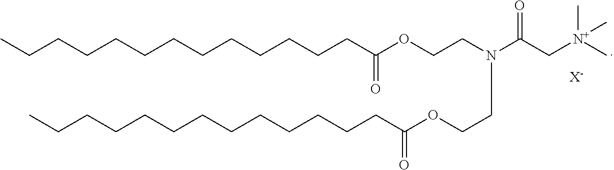

34. A composition comprising a plurality of lipid nanoparticles, wherein the lipid nanoparticles comprise an agent, an ionizable lipid, and a cationic lipid, wherein the cationic lipid is a lipid according to formula VI:

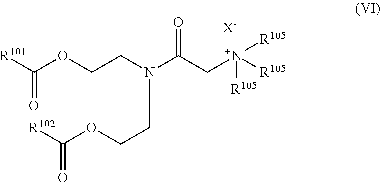

wherein:
R¹⁰¹ and R¹⁰² are each independently optionally substituted (C₈-C₂₄) alkyl or optionally substituted (C₈-C₂₄) alkenyl;
R¹⁰⁵ are each independently (C₁-C₆) alkyl; and
X⁻ is a monovalent anion.
35. The composition of embodiment 34 wherein the cationic lipid is

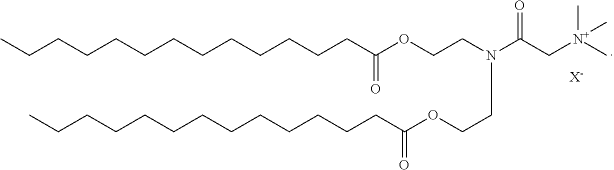

36. A composition comprising a plurality of lipid nanoparticles, wherein the lipid nanoparticles comprise an agent, an ionizable lipid, and a cationic lipid, wherein the ionizable lipid is a lipid according to formula I or a pharmaceutically acceptable salt thereof:

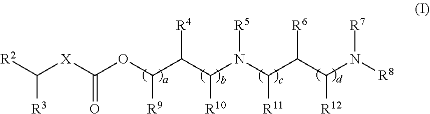

wherein
X is —NR¹— or —O—;
R¹ is a hydrogen atom, a hydrocarbon group having 6 to 24 atoms, or R²¹-L¹-R²²—;
R²¹ is a hydrocarbon group having 1 to 24 carbon atoms;
L¹ is —O(CO)O—, —O(CO)—, —(CO)O—, —O—, or

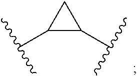 [SMILES: *C1CC1*]

R²² is a divalent hydrocarbon linking group having 1 to 18 carbon atoms;
R² and R³ are each independently a hydrogen atom, a hydrocarbon group having 3 to 24 carbon atoms, or R³¹-L¹-R³²—;
R³¹ is a hydrocarbon group having 1 to 24 carbon atoms;
L² is —O(CO)O—, —O(CO)—, —(CO)O—, —O—, or

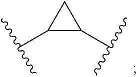 [SMILES: *C1CC1*]

R³² is a divalent hydrocarbon linking group having 1 to 18 carbon atoms;
R⁴, R⁵, R⁶, R⁷, R⁸, R⁹, R¹⁰, R¹¹, and R¹² are each independently a hydrogen atom or an alkyl group having 1 to 18 carbon atoms optionally substituted with R³³, where one or more pairs of groups selected from R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸ may be linked to each other to form a 4- to 7-membered ring of carbon and nitrogen atoms which may contain an oxygen atom;
R³³ is a hydroxyl group, a carboxyl group, —NR⁴⁵R⁴⁶, —O(CO)O—R⁴¹, —O(CO)—R⁴², —(CO)O—R⁴³, —O—R⁴⁴, or an aryl or heteroaryl group optionally substituted with R³⁴,
R³⁴ is an alkyl group having 1 to 18 carbon atoms, a hydroxyl group, a carboxyl group, —NR⁴⁵R⁴⁶, —O(CO)O—R⁴¹, —O(CO)—R⁴², —(CO)O—R⁴³, or —O—R⁴⁴;
R⁴¹, R⁴², R⁴³, R⁴⁴, R⁴⁵, and R⁴⁶ are each independently a hydrocarbon group having 1 to 18 carbon atoms; and
a, b, c, and d are each independently an integer from 0 to 3, where a+b is 1 or more and c+d is 1 or more.
37. The composition of embodiment 36, wherein the ionizable lipid is a lipid according to formula III or a pharmaceutically acceptable salt thereof:

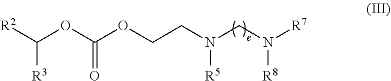

wherein
R² and R³ are each independently a hydrogen atom, a hydrocarbon group having 3 to 24 carbon atoms, or R³¹-L²-R³²—;
R³¹ is a hydrocarbon group having 1 to 24 carbon atoms;
L² is —O(CO)O—, —O(CO)—, —(CO)O—, —O—, or

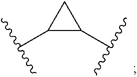 [SMILES: *C1CC1*]

R³² is a divalent hydrocarbon linking group having 1 to 18 carbon atoms;
R⁵ is a hydrogen atom or an alkyl group having 1 to 18 carbon atoms optionally substituted with R³³;
R³³ is a hydroxyl group, a carboxyl group, —NR⁴⁵R⁴⁶, —O(CO)O—R⁴¹, —O(CO)—R⁴², —(CO)O—R⁴³, —O—R⁴⁴, or an aryl or heteroaryl group optionally substituted with R³⁴;
R⁷ and R⁸ are each independently a hydrogen atom or an alkyl group having 1 to 18 carbon atoms optionally substituted with R³³;
R³⁴ is an alkyl group having 1 to 18 carbon atoms, a hydroxyl group, a carboxyl group, —NR⁴⁵R⁴⁶, —O(CO)O—R⁴¹, —O(CO)—R⁴², —(CO)O—R⁴³, or —O—R⁴⁴;
R⁴¹, R⁴², R⁴³, R⁴⁴, R⁴⁵, and R⁴⁶ are each independently a hydrocarbon group having 1 to 18 carbon atoms; and
e is an integer selected from 2 or 3.
38. The composition of embodiment 36 or 37, wherein the ionizable lipid is selected from the group consisting of

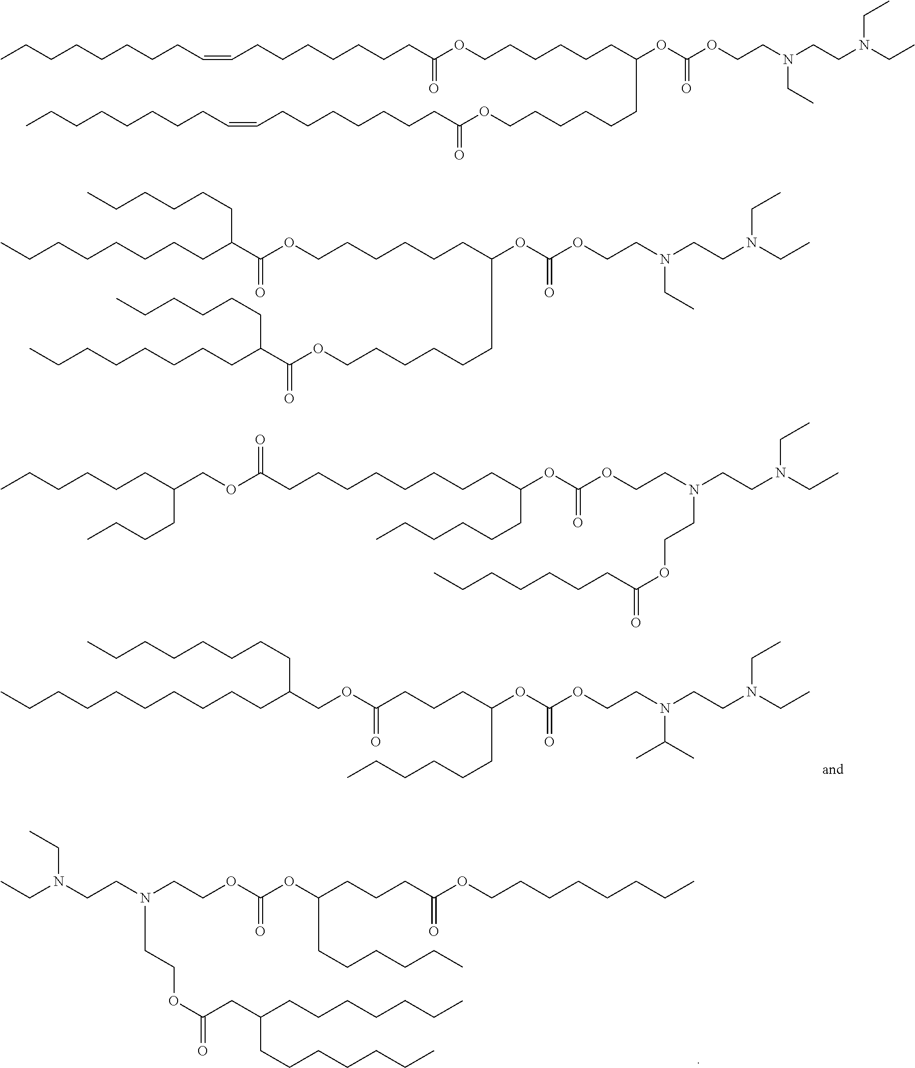

39. A composition comprising a plurality of lipid nanoparticles, wherein the lipid nanoparticles comprise an agent, an ionizable lipid, and a cationic lipid, wherein the ionizable lipid is a lipid according to formula VII or a pharmaceutically acceptable salt thereof:

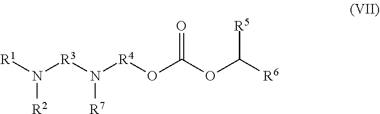

wherein
R¹ and R² are each independently a hydrocarbon group having 1 to 18 carbon atoms, and R³ is a hydrocarbon group having 2 to 8 carbon atoms, wherein the hydrocarbon groups represented by R¹, R², and R³ are each independently optionally substituted with one or more substituents selected from —OH, COOH, —NR⁵¹R⁵², —OC(O)O—R⁵³, —C(O)O—R⁵⁴, —OC(O)—R⁵⁵, and —O—R⁵⁶;
R⁴ is a hydrocarbon group having 1 to 8 carbon atoms;
R⁵ and R⁶ are each independently a hydrocarbon group having 1 to 18 carbon atoms or —R⁸-L¹-R⁹, excluding a case where both R⁵ and R⁶ are hydrocarbon groups having 1 to 8 carbon atoms;
R⁷ is —R¹⁰-L²-R¹¹-L³-R¹²;
R⁵¹ and R⁵² are each independently a hydrocarbon group having 1 to 8 carbon atoms;
R¹⁴, R⁵³, R⁵⁴, R⁵⁵, and R⁵⁶ are each independently a hydrocarbon group having 1 to 24 carbon atoms;
the hydrocarbon groups represented by R⁵³, R⁵⁴, R⁵⁵, and R⁵⁶ are each independently optionally substituted with an aryl group having 6 to 20 carbon atoms or —S—R⁵⁸, wherein the aryl group having 6 to 20 carbon atoms is optionally substituted with —OH, COOH, —NR⁵¹R⁵², —OC(O)O—R⁵³, —C(O)O—R⁵⁴, —OC(O)—R⁵⁵, —O—R⁵⁶, or —R⁵⁹—R⁵⁷, wherein
R⁵⁸ and R⁵⁹ are each independently a hydrocarbon group having 1 to 12 carbon atoms;
R⁵⁷ is —OH, COOH, —NR⁶¹R⁶², —OC(O)O—R⁶³, —C(O)O—R⁶⁴, —OC(O)—R⁶⁵, or —O—R⁶⁶.
R⁶¹ and R⁶² are each independently a hydrocarbon group having 1 to 8 carbon atoms;
R⁶³, R⁶⁴, R⁶⁵, and R⁶⁶ are each independently a hydrocarbon group having 1 to 24 carbon atoms;
the hydrocarbon groups represented by R⁶³, R⁶⁴, R⁶⁵, and R⁶⁶ are each independently optionally substituted with an aryl group having 6 to 20 carbon atoms or —S—R⁶⁸ wherein the aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or —R⁶⁹-R⁶⁷;
R⁶⁸ and R⁶⁹ are each independently a hydrocarbon group having 1 to 12 carbon atoms;
R⁶⁷ is —OH, COOH, —NR⁶¹R⁶², —OC(O)O—R⁶³, —C(O)O—R⁶⁴, —OC(O)—R⁶⁵, or —O—R⁶⁶.
L¹, L², and L³ are each independently —OC(O)O—, —C(O)O—, —OC(O)—, or —O—;

R⁸ is a hydrocarbon group having 1 to 12 carbon atoms;
R⁹ is a hydrocarbon group having 1 to 24 carbon atoms;
R¹⁰ is a hydrocarbon group having 1 to 8 carbon atoms;
R¹¹ is a hydrocarbon group having 1 to 24 carbon atoms;
R¹² is a hydrocarbon group having 1 to 24 carbon atoms;
the hydrocarbon groups represented by R⁹ and R¹² are each independently optionally substituted with an aryl group, —OC(O)O—R⁵³, —C(O)O—R⁵⁴, —OC(O)—R⁵⁵, or —S—R⁵⁸; and
the hydrocarbon groups represented by R¹¹ and R¹⁴ are each independently optionally substituted with —OC(O)O—R⁵³, —C(O)O—R⁵⁴, or —OC(O)—R⁵⁵.
40. The composition of embodiment 39, wherein the ionizable lipid is selected from the group consisting of

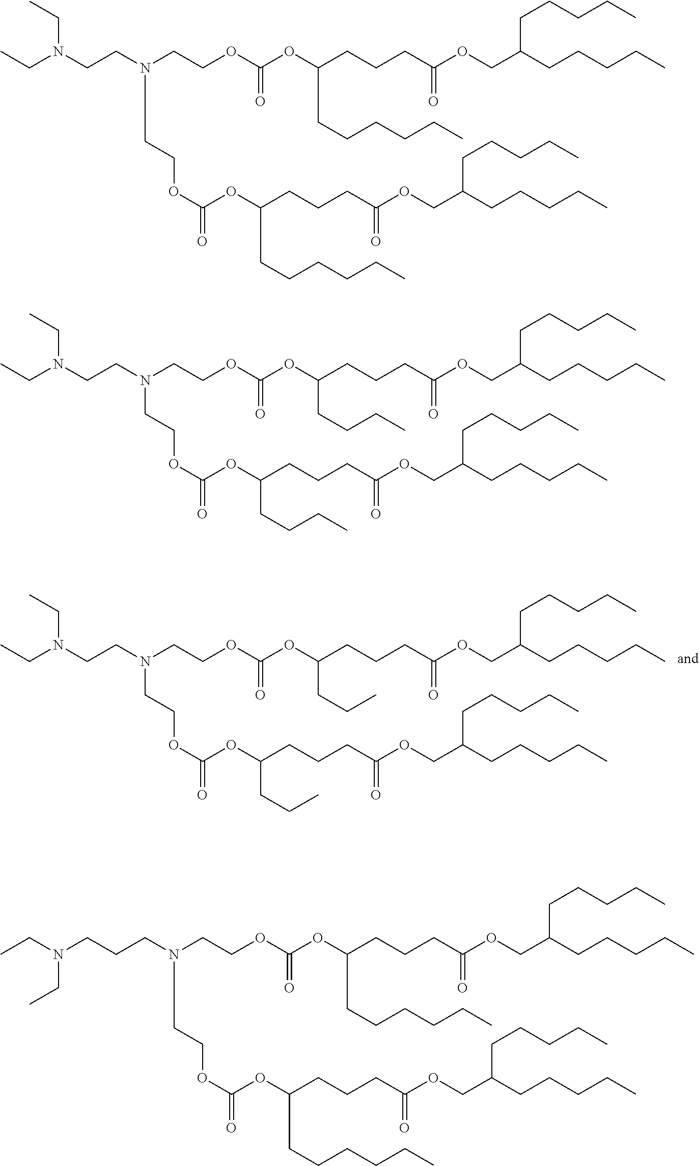

41. A composition comprising a plurality of lipid nanoparticles, wherein the lipid nanoparticles comprise an agent, an ionizable lipid, and at least one of EDPPC and DPTAP, wherein the ionizable lipid is a lipid according to formula I or II or a pharmaceutically acceptable salt thereof.

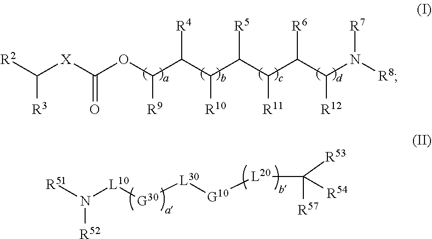

wherein
X is —NR¹— or —O—;
R¹ is a hydrogen atom, a hydrocarbon group having 6 to 24 atoms, or R²¹-L¹-R²²—;
R²¹ is a hydrocarbon group having 1 to 24 carbon atoms;
L¹ is —O(CO)O—, —O(CO)—, —(CO)O—, —O—, or

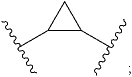 [SMILES: *C1CC1*]

R²² is a divalent hydrocarbon linking group having 1 to 18 carbon atoms;
R² and R³ are each independently a hydrogen atom, a hydrocarbon group having 3 to 24 carbon atoms, or R³¹-L¹-R³²—;
R³¹ is a hydrocarbon group having 1 to 24 carbon atoms;
L² is —O(CO)O—, —O(CO)—, —(CO)O—, —O—, or

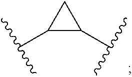 [SMILES: *C1CC1*]

R³² is a divalent hydrocarbon linking group having 1 to 18 carbon atoms;
R⁴, R⁵, R⁶, R⁷, R⁸, R⁹, R¹⁰, R¹¹, and R¹² are each independently a hydrogen atom or an alkyl group having 1 to 18 carbon atoms optionally substituted with R³³, where one or more pairs of groups selected from R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸ may be linked to each other to form a 4- to 7-membered ring of carbon and nitrogen atoms which may contain an oxygen atom;
R³³ is a hydroxyl group, a carboxyl group, —NR⁴⁵R⁴⁶, —O(CO)O—R⁴¹, —O(CO)—R⁴², —(CO)O—R⁴³, —O—R⁴⁴, or an aryl or heteroaryl group optionally substituted with R³⁴;
R³⁴ is an alkyl group having 1 to 18 carbon atoms, a hydroxyl group, a carboxyl group, —NR⁴⁵R⁴⁶, —O(CO)O—R⁴¹, —O(CO)—R⁴², —(CO)O—R⁴³, or —O—R⁴⁴;
R⁴¹, R⁴², R⁴³, R⁴⁴, R⁴⁵, and R⁴⁶ are each independently a hydrocarbon group having 1 to 18 carbon atoms;
a, b, c, and d are each independently an integer from 0 to 3, where a+b is 1 or more and c+d is 1 or more;
R⁵¹ and R⁵² are each independently an alkyl group having 1 to 18 carbon atoms optionally substituted with R³⁵,
R³⁵ is a hydroxyl group, -G²⁰-CH(R⁵⁵)(R⁵⁶), —N(R⁵⁸)(R⁵⁹), or -G²⁰-R⁶⁰;
G²⁰ is —(CO)O— or —O(CO)—;
R⁵⁵ and R⁵⁶ are each independently hydrogen or an alkyl group having 1 to 18 carbon atoms;
R⁵⁸ and R⁵⁹ are each independently hydrogen or a cycloalkyl group having 3 to 6 carbon atoms, optionally substituted with R³⁶,
R⁶⁰ is alkyl group having 1 to 18 carbon atoms or a cycloalkyl group having 3 to 6 carbon atoms;
R³⁶ is —N(R⁶¹)(R⁶²), or -G²⁰-R⁶⁵;
R⁶¹ and R⁶² are each independently hydrogen or a cycloalkyl group having 3 to 6 carbon atoms;
R⁶⁵ is an alkyl group having 1 to 18 carbon atoms, -L⁴⁰-CH(R⁶⁶)(R⁶⁷), or -G²⁰-R⁶⁶,
L⁴⁰ is a divalent alkyl group containing 1 to 6 carbon atoms;
R⁶⁶ and R⁶⁷ are each independently an alkyl group containing 1 to 10 carbon atoms or an alkoxy group containing 1 to 10 carbon atoms;
L¹⁰ is a divalent alkyl group containing 1 to 10 carbon atoms;
G³⁰ is —S(CO)N(R⁶⁴);
R⁶⁴ is -L³⁰-G²⁰-CH(R⁵⁵)(R⁵⁶);
a′ is 0 or 1;
G¹⁰ is G²⁰, —O(CO)O—, or —N(R⁶³)C(O)—;
R⁶³ is an alkyl group containing 1 to 18 carbon atoms;
L²⁰ is a divalent alkyl group containing 1 to 6 carbon atoms;
b′ is 0 or 1;
R⁵³, R⁵⁴, and R⁵⁷ are each independently hydrogen or an alkyl group containing 1 to 18 carbon atoms optionally substituted with R³⁶, and
L³⁰ is a single bond or an alkyl group containing 1 to 18 carbon atoms.
42. The composition of any one of embodiments 34-41, wherein the agent is a bioactive agent.
43. The composition of embodiment 42, wherein the bioactive agent is a therapeutic agent.
44. The composition of embodiment 43, wherein the therapeutic agent is a nutraceutical agent.
45. The composition of any one of embodiments 34-41, wherein the agent is an imaging agent.
46. The composition of any one of embodiments 34-41, wherein the agent is a diagnostic agent.
47. The composition of any one of embodiments 34-41, wherein the agent is a nucleic acid.
48. The composition of embodiment 47, wherein the nucleic acid is a ribonucleic acid.
49. The composition of embodiment 47, wherein the nucleic acid is a deoxyribonucleic acid.
50. The composition of embodiment 47, wherein the nucleic acid is a non-natural nucleic acid.
51. A method of delivering an agent to one or more of the brain, heart, muscles, and kidneys of a subject in need thereof, comprising systemically administering to the subject a composition comprising a plurality of lipid nanoparticles, wherein the lipid nanoparticles comprise the agent, an ionizable lipid, and a fully saturated cationic lipid.
52. The method of embodiment 51, wherein the agent is delivered to the brain.
53. The method of embodiment 51, wherein the agent is delivered to the heart.
54. The method of embodiment 51, wherein the agent is delivered to the muscles.
55. The method of embodiment 51, wherein the agent is delivered to the kidneys.
56. The method of any one of embodiments 51-55, wherein the ionizable lipid is a lipid of formula (I).
57. The method of any one of embodiments 51-56, wherein the ionizable lipid is a lipid of formula (III).
58. The method of any one of embodiments 51-55, wherein the ionizable lipid is a lipid of formula (II).
59. The method of any one of embodiments 51-55, wherein the ionizable lipid is a lipid of formula (VII).
60. The method of any one of embodiments 51-59, wherein the cationic lipid is a lipid of formula (IV).
61. The method of any one of embodiments 51-59, wherein the cationic lipid is a lipid of formula (V).
62. The method of any one of embodiments 51-59, wherein the cationic lipid is a lipid of formula (VI).
63. A method of delivering an agent to a subject in need thereof, comprising administering to the subject a composition of any one of embodiments 1-33.
64. A method of delivering an agent to a subject in need thereof, comprising administering to the subject a composition of any one of embodiments 34-35.
65. A method of delivering an agent to a subject in need thereof, comprising administering to the subject a composition of any one of embodiments 36-38.
66. A method of delivering an agent to a subject in need thereof, comprising administering to the subject a composition of any one of embodiments 39-50.
67. A method of treating a subject having a disease, disorder or condition beneficially treated by an agent, comprising administering to the subject a therapeutically effective amount of a composition of any one of embodiment 1-50.

### C. Lipid Particles

In some embodiments, the lipid particles of the present disclosure comprise an ionizable lipid, a cationic lipid, and an agent. In some embodiments, the lipid particles further comprise a sterol. In some embodiments, the lipid particles further comprise a phospholipid. In some embodiments, the lipid particles further comprise a PEG-lipid. Exemplary and preferred lipids of each type are as provided herein. In some embodiments, the molar percent distribution for the ionizable lipid and cationic lipid is 10-90:10-90, respectively, wherein the sum of each molar percent must be 100. In some embodiments, the molar percent distribution for the ionizable lipid, cationic lipid, and sterol is 10-90:10-90:10-90, respectively, wherein the sum of each molar percent must be 100. In some embodiments, the molar percent distribution for the ionizable lipid, cationic lipid, and sterol is 15-75:5-75:25-80, respectively, wherein the sum of each molar percent must be 100. In some embodiments, the molar percent distribution for the ionizable lipid, cationic lipid, and sterol is 20-60:15-50:20-60, respectively, wherein the sum of each molar percent must be 100. In some embodiments, the molar percent distribution for the ionizable lipid, cationic lipid, sterol, and phospholipid is 15-75:5-75:25-80:0.1-10, respectively, wherein the sum of each molar percent must be 100. In some embodiments, the molar percent distribution for the ionizable lipid, cationic lipid, sterol, and phospholipid is 20-60:15-50:20-60:2.5-7.5, respectively, wherein the sum of each molar percent must be 100. In some embodiments, the molar percent distribution for the ionizable lipid, cationic lipid, sterol, phospholipid, and PEG lipid is 20-60:15-50:20-60:2.5-7.5:0.5-2.5, respectively, wherein the sum of each molar percent must be 100. In some embodiments, the molar percent distribution for the ionizable lipid, cationic lipid, sterol, and PEG lipid is 20-60:15-50:20-60:0.5-2.5, respectively, wherein the sum of each molar percent must be 100.

As used herein “lipid particles” refers to particles composed of at least one lipid. Lipid particles may have a variety of structures, for example, a lipid particle may be in the form of a lipid aggregate, a micelle, a liposome, a lipoplex, a lipid nanoparticle, and the like in accordance with the present disclosure. For example, in some embodiments, a lipid particle has a single lipid bilayer and an internal aqueous phase. In other embodiments, the lipid particle is a liposome with multiple lipid bilayers stacked together with multiple internal aqueous phase sections. The bilayers of lipid particles of the present disclosure may be substantially gel-like, substantially fluid, or substantially solid. In some embodiments, the phase of the bilayer may change (e.g., by melting). This phase change can be due to, for example, temperature change, pH change, composition change, storage, administration, solvent change or any other factor.

In some embodiments, the lipid particle is a lipid nanoparticle. As used herein, “lipid nanoparticle” refers to a nanoparticle comprising a plurality of lipid molecules. Further, a “nanoparticle” refers to a particle that is 1 to 1000 nm in diameter, but is particularly 10 to 1000 nm. More particularly the particle is 30 to 500 nm in diameter, even more particularly 50 to 250 nm, particularly particularly 50 to 200 nm, and most particularly 50 to 150 nm.

In some embodiments, the agent is contained within an internal aqueous phase of a lipid particle. In other embodiments, the agent is bound on the surface of a lipid particle (covalently or non-covalently). In some embodiments, all of or a portion of the agent is embedded in a lipid bilayer of a lipid particle. In some embodiments, the agent is covalently bound to a lipid particle.

In some embodiments, the agent is a bioactive agent. Examples of bioactive agents include pharmaceutical compounds (e.g. small molecules), nucleic acids, oligonucleotides, dietary supplements, proteins, peptides, amino acids, and the like. In some embodiments the agent is a therapeutic agent.

In some embodiments the agent is a nucleic acid. Nucleic acid agents can be double-stranded or single-stranded. In addition, nucleic acid agents can be linear or circular nucleic acid molecules. In some embodiments the nucleic acid is DNA, RNA, or a non-natural nucleic acid. The RNA agent may be RNA encoding a peptide or a non-coding RNA. The RNA agent may be mRNA, siRNA, miRNA, an enhancer RNA, long noncoding RNA, and the like. The nucleic acid may regulate gene expression, encode a peptide, or be an aptamer, among other possibilities. In some embodiments, a nucleic acid has a length of about 15 to about 30 nucleotides, for example, about 20-25 nucleotides (e.g., 20, 21, 22, 23, 24 or 25 nucleotides). In some embodiments, a nucleic acid has a length of greater than about 30 nucleotides, such as, for example, about 30, 35, 40, 45, 50, 60, 70, 80, 90, 100, 150, 200, 250, 300, 400, 500, 750, 1000 or more nucleotides.

In some embodiments the agent is a nutraceutical agent, such as, for example, a vitamin, an antioxidant, or a dietary supplement.

In some embodiments the agent is an imaging agent. Examples of imaging agents include but are not limited to chemical or biological dyes, fluorescent probes, radioactive isotopes, spin-labeled tracers, and the like.

The lipid particles of the present disclosure may comprise one or more agents. (e.g., 2, 3, 4, 5, 6, 7, 8, 9, or 10 agents).

As used herein, “zeta potential” refers to the electrical potential at the interface of the bulk solution and the stationary solvent and solutes associated with the particle. The zeta potential reflects the degree of electrostatic repulsion between particles in solution. As a result, the zeta potential of a lipid particle can be used as a predictor of the stability of the particles in solution (e.g., resistance to coagulation or aggregation). In some embodiments the lipid particles of the present disclosure have a zeta potential of less than −20 millivolts. In some embodiments the lipid particles of the present disclosure have a zeta potential of less than-10 millivolts. In some embodiments the lipid particles of the present disclosure have a zeta potential of less than −5 millivolts. In some embodiments the lipid particles of the present disclosure have a zeta potential of less than 0 millivolts. In some embodiments the lipid particles of the present disclosure have a zeta potential of less than 5 millivolts. In some embodiments the lipid particles of the present disclosure have a zeta potential of less than 10 millivolts. In some embodiments the lipid particles of the present disclosure have a zeta potential of less than 20 millivolts. In some embodiments the lipid particles of the present disclosure have a zeta potential of more than −20 millivolts. In some embodiments the lipid particles of the present disclosure have a zeta potential of more than-10 millivolts. In some embodiments the lipid particles of the present disclosure have a zeta potential of more than-5 millivolts. In some embodiments the lipid particles of the present disclosure have a zeta potential of more than 0 millivolts. In some embodiments the lipid particles of the present disclosure have a zeta potential of more than 5 millivolts. In some embodiments the lipid particles of the present disclosure have a zeta potential of more than 10 millivolts. In some embodiments the lipid particles of the present disclosure have a zeta potential of more than 20 millivolts. In some embodiments the lipid particles have a zeta potential between −20 and 20 millivolts. In some embodiments the lipid particles have a zeta potential between −10 and 20 millivolts. In some embodiments the lipid particles have a zeta potential between 0 and 20 millivolts. In some embodiments the lipid particles have a zeta potential between −20 and 10 millivolts. In some embodiments the lipid particles have a zeta potential between −20 and 0 millivolts. In some embodiments the lipid particles have a zeta potential between −10 and 10 millivolts. In some embodiments the lipid particles have a zeta potential between 0 and 10 millivolts. In some embodiments the lipid particles have a zeta potential between 5 and 10 millivolts. In some embodiments, the zeta potential is as measured by light scattering, e.g., using a Zetasizer Nano ZS (Malvern Instruments) in a 0.1X PBS solution, e.g., as described in the Exemplification.

As used herein “pKa” refers to the apparent ionization constant, e.g., of a lipid nanoparticle. The pKa of a lipid nanoparticle of the present disclosure may be between about 4 and about 9. In some embodiments the pKa of a lipid nanoparticle of the present disclosure is between about 5 and about 8. In some embodiments the pKa of a lipid nanoparticle of the present disclosure is between about 5.5 and about 7. Preferably, the pKa of a lipid nanoparticle of the present disclosure is between about 6 and about 7. In some embodiments the pKa is as measured using the 2-(p-toluidinyl) naphthalene-6-sulphonic acid (TNS) assay, e.g., as described in Heyes J. et al. Journal of Controlled Release 107 (2005) 276-287 and the Exemplification.

As used herein, encapsulation efficiency refers to the amount of an agent encapsulated within particles of a composition, expressed as a percent of the total amount of agent. In some embodiments the encapsulation efficiency is greater than 50%. In some embodiments the encapsulation efficiency is greater than 60%. In some embodiments the encapsulation efficiency is greater than 70%. In some embodiments the encapsulation efficiency is greater than 80%. In some embodiments the encapsulation efficiency is greater than 90%. In some embodiments is greater than 91%. In some embodiments the encapsulation efficiency is greater than 93%. In some embodiments the encapsulation efficiency is greater than 95%. In some embodiments the encapsulation efficiency is greater than 97%. In some embodiments the encapsulation efficiency is greater than 98%. In some embodiments the encapsulation efficiency is greater than 99%. In some embodiments, the encapsulation efficiency is as determined by the modified Quant-iT RiboGreen RNA assay (Thermo Fisher), e.g., as described in Walsh C. et al. Methods Mol Biol. 2014; 1141:109-20 and the Exemplification.

As used herein “polydispersity index,” “PDI,” and “dispersity” are used interchangeably, and are measures of the heterogeneity in size of lipid particles in a population of lipid particles. Lower values indicate more uniform size distributions. In some embodiments, the PDI of the lipid particles in a composition described herein are less than 0.5. In some embodiments, the PDI of the lipid particles in a composition described herein are less than 0.3. In some embodiments, the PDI of the lipid particles in a composition described herein are less than 0.2. In some embodiments, the PDI of the lipid particles in a composition described herein are less than 0.15. In some embodiments, the PDI of the lipid particles in a composition described herein are less than 0.1. In some embodiments, the PDI of the lipid particles in a composition described herein are less than 0.08. In some embodiments, the PDI of the lipid particles in a composition described herein are less than 0.07. In some embodiments, the PDI of the lipid particles in a composition described herein are less than 0.06. In some embodiments, the PDI of the lipid particles in a composition described herein are less than 0.05. In some embodiments PDI is as measured by dynamic light scattering, e.g., using a Zetasizer Nano ZS (Malvern Instruments), e.g. as in the Exemplification.

As used herein, the “Z-average” refers to the intensity weighted average diameter of a lipid nanoparticle in a composition described herein. In some embodiment, the Z-average particle diameter of a lipid nanoparticle of the present disclosure may be between 10 and 500 nm. In some embodiments the Z-average particle diameter of a lipid nanoparticle of the present disclosure may be between 10 and 300 nm. In some embodiments the Z-average particle diameter of a lipid nanoparticle of the present disclosure may be between 10 and 200 nm. In some embodiments the Z-average particle diameter of a lipid nanoparticle of the present disclosure may be between 10 and 150 nm. In some embodiments the Z-average particle diameter of a lipid nanoparticle of the present disclosure may be between 10 and 100 nm. In some embodiments the Z-average particle diameter of a lipid nanoparticle of the present disclosure may be less than 300 nm. In some embodiments the Z-average particle diameter of a lipid nanoparticle of the present disclosure may be less than 250 nm. In some embodiments the Z-average particle diameter of a lipid nanoparticle of the present disclosure may be less than 200 nm. In some embodiments the Z-average particle diameter of a lipid nanoparticle of the present disclosure may be less than 150 nm. In some embodiments the Z-average particle diameter of a lipid nanoparticle of the present disclosure may be less than 100 nm. In some embodiments the Z-average particle diameter of a lipid nanoparticle of the present disclosure is as determined by dynamic light scattering, e.g., measured using a Zetasizer Nano ZS (Malvern Instruments), e.g. as in the Exemplification.

### D. Ionizable Lipids

In some embodiments of the composition of the present disclosure, the composition comprises an ionizable lipid. In some embodiments, the ionizable lipids contain one or more groups which is ionic at physiological pH but may have no charge at a certain pH outside of the range of physiological pH values. Typically, the ionizable lipids of the present disclosure are cationic ionizable lipids which have a cationic charge at physiological pH values. The ionizable cationic group may contain one or more protonatable amines which are able to form a cationic group at physiological pH. The ionizable lipid compound may also further comprise one or more lipid components such as two or more fatty acids with C₁-C₃₀ alkyl or alkenyl carbon groups. In some embodiments of the composition of the present application, the ionizable cationic lipids refer to lipid and lipid-like molecules with nitrogen atoms that can acquire charge (pKa). These molecules with amino groups typically have between 2 and 6 hydrophobic chains, often alkyl or alkenyl such as C₁-C₃₀ alkyl or alkenyl groups, but may have at least 1 or more than 6 tails.

The ionizable lipid component of the lipid nanoparticle of the present invention may comprise of between 1 and 99 molar percent of the lipid molecules in the nanoparticle. In other embodiments, the ionizable lipid component may comprise of between 5 and 90 molar percent of the lipid molecules in the lipid nanoparticles. In some embodiments the ionizable lipid component comprises between 10 and 75, 20 and 75, 20 and 60, or 20 and 55 molar percent of the lipid molecules in the lipid nanoparticles.

The ionizable lipids of the present application may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Ionizable lipids may occur as racemates and racemic mixtures, single enantiomers, diastereomeric mixtures and individual diastereomers. In some embodiments, a single diastereomer is obtained. The chiral centers of the cationic ionizable lipids of the present application can have the S or the R configuration.

The ionizable lipids of the present application may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In some embodiments, the ionizable cationic lipid comprises an ammonium group which is positively charged at physiological pH and contains at least two hydrophobic groups. In some embodiments, the ammonium group is positively charged at a pH from about 6 to about 8. In some embodiments, the ionizable cationic lipid comprises at least two C₁-C₃₀ alkyl or alkenyl groups.

In some embodiments, an ionizable lipid suitable for use in the present disclosure is biodegradable. As used herein “biodegradable” refers to the quality of a lipid or other compound to be broken into smaller groups that are then metabolized, catabolized, excreted, and/or otherwise removed from the body by natural chemical and biological processes within the subject. The biodegradation may take place by chemical means without the action of an enzyme, such as an ester breaking down into a carboxylate and an alcohol in a high- or low-pH environment. In other embodiments, the biodegradation is catalyzed by an enzyme, such as the breakdown of esters by esterase enzymes. Certain groups are more easily or rapidly biodegraded than others and contribute to how quickly a larger molecule such as a lipid or lipid particle will biodegrade. The incorporation of biodegradable group(s) into the ionizable lipid results in faster metabolism and removal of the ionizable lipid from the body following delivery of the active agent to a target area. As a result, these ionizable lipids may have lower toxicity than similar ionizable lipids without the biodegradable groups.

Examples of biodegradable groups suitable for use in the present disclosure include, but are not limited to: —OC(O), —C(O)O—, —SC(O)—, —C(O)S—, —OC(S)—, —C(S)O—, —S—S—, —C(Z³)═N—,
—N═C(Z³), —C(Z³)═N—O—, —O—N═C(Z³)—, —C(O) (NZ³)—, —N(Z³)C(O)—, —C(S)(NZ³)—, —N(Z³) C(O)—,
—N(Z³)C(O)N(Z³)—, —OC(O)O—, —OSi(Z³)₂O—, —C(O)(CZ¹Z²)C(O)O—, or —OC(O)(CZ¹Z²)C(O)—; wherein Z¹ and Z² are independently H, OH, alkyl, alkoxy, —NH₂, alkylamino, or dialkylamino and Z³ is H or alkyl. Typically, a compound containing more biodegradable groups is itself said to be more biodegradable. In some embodiments, a biodegradable lipid of the present disclosure contains two or more biodegradable groups. In some embodiments, a biodegradable lipid of the present disclosure contains three or more biodegradable groups. In some embodiments, a biodegradable lipid of the present disclosure contains four or more biodegradable groups. In some embodiments, a lipid is considered biodegradable despite not having any of the biodegradable groups listed above.

In certain embodiments the ionizable lipid for use in the present disclosure is a lipid of formula (I) or a pharmaceutically acceptable salt thereof:

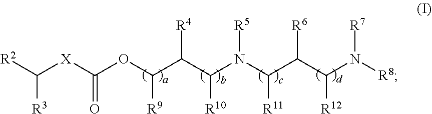

wherein
X is —NR¹— or —O—;
R¹ is a hydrogen atom, a hydrocarbon group having 6 to 24 atoms, or R²¹-L¹-R²²—;
R²¹ is a hydrocarbon group having 1 to 24 carbon atoms;

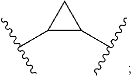 [SMILES: *C1CC1*]

L¹ is —O(CO)O—, —O(CO)—, —(CO)O—, —O—, or
R²² is a divalent hydrocarbon linking group having 1 to 18 carbon atoms;
R² and R³ are each independently a hydrogen atom, a hydrocarbon group having 3 to 24 carbon atoms, or R³¹-L¹-R³²—;
R³¹ is a hydrocarbon group having 1 to 24 carbon atoms;
L² is —O(CO)O—, —O(CO)—, —(CO)O—, —O—, or

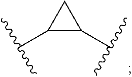 [SMILES: *C1CC1*]

R³² is a divalent hydrocarbon linking group having 1 to 18 carbon atoms;
R⁴, R⁵, R⁶, R⁷, R⁸, R⁹, R¹⁰, R¹¹, and R¹² are each independently a hydrogen atom or an alkyl group having 1 to 18 carbon atoms optionally substituted with R³³, where one or more pairs of groups selected from R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸ may be linked to each other to form a 4- to 7-membered ring of carbon and nitrogen atoms which may contain an oxygen atom;
R³³ is a hydroxyl group, a carboxyl group, —NR⁴⁵R⁴⁶, —O(CO)O—R⁴¹, —O(CO)—R⁴², —(CO)O—R⁴³, —O—R⁴⁴, or an aryl or heteroaryl group optionally substituted with R³⁴,
R³⁴ is an alkyl group having 1 to 18 carbon atoms, a hydroxyl group, a carboxyl group, —NR⁴⁵R⁴⁶, —O(CO)O—R⁴¹, —O(CO)—R⁴², —(CO)O—R⁴³, or —O—R⁴⁴,
R⁴¹, R⁴², R⁴³, R⁴⁴, R⁴⁵, and R⁴⁶ are each independently a hydrocarbon group having 1 to 18 carbon atoms; and
a, b, c, and d are each independently an integer from 0 to 3, where a+b is 1 or more and c+d is 1 or more.

For the hydrocarbon group having 6 to 24 carbon atoms from R¹ and the hydrocarbon group having 3 to 24 carbon atoms from R² and R³, an alkyl group, an alkenyl group, or an alkynyl group is preferable, and an alkyl group or an alkenyl group is more preferable. An alkyl group may be linear or branched or may be chainlike or cyclic. An alkyl group having 6 to 24 carbon atoms is preferably an alkyl group having 6 to 20 carbon atoms, and an alkyl group having 3 to 24 carbon atoms is more preferably an alkyl group having 6 to 20 carbon atoms. Specific examples thereof include a hexyl group, a heptyl group, an octyl group, a nonyl group, a decyl group, an undecyl group, a dodecyl group, a tridecyl group, a trimethyldodecyl group (preferably a 3,7,11-trimethyldodecyl group), a tetradecyl group, a pentadecyl group, a hexadecyl group, a tetramethylhexadecyl group (preferably a 3,7,11,15-tetramethylhexadecyl group), a heptadecyl group, an octadecyl group, a nonadecyl group, an icosyl group, and the like. An alkenyl group having 6 to 24 carbon atoms and an alkenyl group having 3 to 24 carbon atoms may be linear or branched or may be chainlike or cyclic. An alkenyl group having 6 to 24 carbon atoms is preferably an alkenyl group having 6 to 20 carbon atoms, and an alkenyl group having 3 to 24 carbon atoms is more preferably an alkenyl group having 6 to 20 carbon atoms. Specific examples thereof include a hexenyl group, a heptenyl group, an octenyl group, a nonenyl group, a decenyl group, an undecenyl group, a dodecenyl group, a dodecadienyl group, a tridecenyl group, a tetradecenyl group, a pentadecenyl group, a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8,11-dienyl group), an octadecenyl group (preferably a (Z)-octadec-9-enyl group), an octadecadienyl group (preferably a (9Z,12Z)-octadeca-9,12-dienyl group), a nonadecenyl group, an icosenyl group (preferably a (Z)-icos-11-enyl group), an icosadienyl group (preferably a (11Z,14Z)-icosa-11,14-dienyl group), and the like. An alkynyl group having 6 to 24 carbon atoms is preferably an alkynyl group having 6 to 20 carbon atoms, and an alkynyl group having 3 to 24 carbon atoms is more preferably an alkynyl group having 6 to 20 carbon atoms. Specifically, examples thereof include a hexynyl group, a heptynyl group, an octynyl group, a nonynyl group, a decynyl group, an undecynyl group, a dodecynyl group, a tetradecynyl group, a pentadecynyl group, a hexadecynyl group, a heptadecynyl group, an octadecynyl group, and the like. All of the above alkenyl groups preferably have one double bond or two double bonds. All of the above alkynyl groups preferably have one triple bond or two triple bonds.

For the hydrocarbon group having 1 to 24 carbon atoms from R²¹ and R³¹, an alkyl group having 10 to 24 carbon atoms, an alkenyl group having 10 to 24 carbon atoms, or an alkynyl group having 10 to 24 carbon atoms is preferable. An alkyl group having 10 to 24 carbon atoms may be linear or branched or may be chainlike or cyclic. An alkyl group having 10 to 24 carbon atoms is preferably an alkyl group having 12 to 24 carbon atoms. Specific examples thereof include a decyl group, an undecyl group, a dodecyl group, a tridecyl group, a trimethyldodecyl group (preferably a 3,7,11-trimethyldodecyl group), a tetradecyl group, a pentadecyl group, a hexadecyl group, a tetramethylhexadecyl group (preferably a 3,7,11,15-tetramethylhexadecyl group), a heptadecyl group, an octadecyl group, a 2-butylhexyl group, a 2-butyloctyl group, a 1-pentylhexyl group, a 2-pentylheptyl group, a 3-pentyloctyl group, a 1-hexylheptyl group, a 1-hexylnonyl group, a 2-hexyloctyl group, a 2-hexyldecyl group, a 3-hexylnonyl group, a 1-heptyloctyl group, a 2-heptylnonyl group, a 2-heptylundecyl group, a 3-heptyldecyl group, a 1-octylnonyl group, a 2-octyldecyl group, a 2-octyldodecyl group, a 3-octylundecyl group, a 2-nonylundecyl group, a 3-nonyldodecyl group, a 2-decyldodecyl group, a 2-decyltetradecyl group, a 3-decyltridecyl group, a 2-(4,4-dimethylpentan-2-yl)-5,7,7-trimethyloctyl group, and the like. An alkenyl group having 10 to 24 carbon atoms may be linear or branched or may be chainlike or cyclic. Specific examples thereof include a decenyl group, an undecenyl group, a dodecenyl group, a dodecadienyl group, tridecenyl group (preferably a (Z)-tridec-8-enyl group), a tetradecenyl group (preferably a tetradec-9-enyl group), a pentadecenyl group (preferably a (Z)-pentadec-8-enyl group), a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8, 11-dienyl group), an octadecenyl group (preferably a (Z)-octadec-9-enyl group), an octadecadienyl group (preferably a (9Z,12Z)-octadeca-9,12-dienyl group), and the like. An alkynyl group having 10 to 24 carbon atoms may be linear or branched or may be chainlike or cyclic. Specific examples thereof include a decynyl group, an undecynyl group, a dodecynyl group, a tetradecynyl group, a pentadecynyl group, a hexadecynyl group, a heptadecynyl group, an octadecynyl group, and the like. All of the above alkenyl groups preferably have one double bond or two double bonds. All of the above alkynyl groups preferably have one triple bond or two triple bonds.

For the divalent hydrocarbon linking group having 1 to 18 carbon atoms from R²² and R³² an alkylene group having 1 to 18 carbon atoms or an alkenylene group having 2 to 18 carbon atoms is preferred. An alkylene group having 1 to 18 carbon atoms may be linear or branched or may be chainlike or cyclic. The number of carbon atoms in an alkylene group is preferably 1 to 12, more preferably 1 to 10, and still more preferably 2 to 10. Specific examples thereof include a methylene group, an ethylene group, a trimethylene group, a tetramethylene group, a pentamethylene group, a hexamethylene group, a heptamethylene group, an octamethylene group, a nonamethylene group, a decamethylene group, an undecamethylene group, a dodecamethylene group, and the like. An alkenylene group having 2 to 18 carbon atoms may be linear or branched or may be chainlike or cyclic. The number of carbon atoms in an alkenylene group is preferably 1 to 12, and more preferably 2 to 10.

Preferred embodiments of L¹ and L² are —O(CO)O—, —O(CO)—, and —(CO)O—. More preferred embodiments of L¹ and L² are —O(CO)— and —(CO)O—.

The optionally substituted alkyl group having 1 to 18 carbon atoms from R⁴, R⁶, R⁹, R¹⁰, R¹¹, and R¹² may be linear or branched or may be chainlike or cyclic. The number of carbon atoms in the alkyl group is preferably 1 to 12. Specific examples thereof include a methyl group, an ethyl group, a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, and the like. In a case where the alkyl group has a substituent, a hydroxyl group, a carboxyl group, or a group represented by —O(CO)O—R⁴¹, —O(CO)—R⁴², —(CO)O—R⁴³, or —O—R⁴⁴ is preferable, and a group represented by —O(CO)—R₄₂ or —(CO)O—R⁴³ is more preferable.

The optionally substituted alkyl group having 1 to 18 carbon atoms from R⁵, R⁷, and R⁸ may be linear or branched or may be chainlike or cyclic. The number of carbon atoms in the alkyl group is preferably 1 to 12, and more preferably 1 to 8. Specific examples thereof include a methyl group, an ethyl group, a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, and the like. In a case where the alkyl group has a substituent, a hydroxyl group, a carboxyl group, or a group represented by —O(CO)O—R⁴¹, —O(CO)—R⁴², —(CO)O—R⁴³, or —O—R⁴⁴ is preferable, and a group represented by —O(CO)—R⁴², —(CO)O—R⁴³, or —O—R⁴⁴ is more preferable.

Examples of the 4- to 7-membered ring of carbon and nitrogen atoms which may contain an oxygen atom include an azetidine ring, a pyrrolidine ring, a piperidine ring, a morpholine ring, and an azepane ring. The 4- to 7-membered ring is preferably a 6-membered ring and is preferably a piperidine ring or a morpholine ring.

When the optionally substituted alkyl group having 1 to 18 carbon atoms from R⁴, R⁵, R⁶, R⁷, R⁸, R⁹, R¹⁰, R¹¹, and R¹² is substituted with a substituted or unsubstituted aryl group, the number of carbon atoms in the aryl group is preferably 6 to 22, more preferably 6 to 18, and still more preferably 6 to 10. Specific examples of the aryl group include a phenyl group, a naphthyl group, an anthracenyl group, a phenanthrenyl group, and the like. For the substituent on the aryl group, an alkyl group having 1 to 18 carbon atoms, a hydroxyl group, a carboxyl group, an amino group represented by —NR⁴⁵R⁴⁶, or a group represented by —O(CO)O—R⁴¹, —O(CO)—R⁴², —(CO)O—R⁴³, or —O—R⁴⁴ is preferable, and a hydroxyl group or a carboxyl group is more preferable. Specific examples of the substituted aryl group include a hydroxyphenyl group, a carboxyphenyl group, and the like.

When the optionally substituted alkyl group having 1 to 18 carbon atoms from R⁴, R⁵, R⁶, R⁷, R⁸, R⁹, R¹⁰, R¹¹, and R¹² is substituted with a substituted or unsubstituted heteroaryl group as a substituent, the number of carbon atoms in the heteroaryl group is preferably 1 to 12, and more preferably 1 to 6. Specific examples of the heteroaryl group include a pyridyl group, a pyrazolyl group, an imidazolyl group, a benzimidazolyl group, a thiazolyl group, an oxazolyl group, and the like. For the substituent on the heteroaryl group, an alkyl group having 1 to 18 carbon atoms, a hydroxyl group, a carboxyl group, an amino group represented by —NR⁴⁵R⁴⁶, or a group represented by —O(CO)O—R⁴¹, —O(CO)—R⁴², —(CO)O—R⁴³, or —O—R⁴⁴ is preferable, and a hydroxyl group or a carboxyl group is more preferable. Specific examples of the substituted or unsubstituted heteroaryl group include a hydroxypyridyl group, a carboxypyridyl group, a pyridonyl group, and the like.

For the hydrocarbon group having 1 to 18 carbon atoms that is represented by R⁴¹, R⁴², R⁴³, R⁴⁴, R⁴⁵, and R⁴⁶, an alkyl group having 1 to 18 carbon atoms, an alkenyl group having 2 to 18 carbon atoms, or an alkynyl group having 2 to 18 carbon atoms is preferable, and an alkyl group having 1 to 18 carbon atoms or an alkenyl group having 2 to 18 carbon atoms is more preferable. The alkyl group having 1 to 18 carbon atoms may be linear or branched or may be chainlike or cyclic. The number of carbon atoms in the alkyl group is preferably 3 to 18, and more preferably 5 to 18. Specific examples thereof include a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, a tridecyl group, a trimethyldodecyl group (preferably a 3,7, 11-trimethyldodecyl group), a tetradecyl group, a pentadecyl group, a hexadecyl group, a heptadecyl group, an octadecyl group, and the like. The alkenyl group having 2 to 18 carbon atoms may be linear or branched or may be chainlike or cyclic. The number of carbon atoms in the alkenyl group is preferably 3 to 18, and more preferably 5 to 18. Specific examples thereof include an allyl group, a prenyl group, a pentenyl group, a hexenyl group, a heptenyl group, an octenyl group, a nonenyl group (preferably a (Z)-2-nonenyl group or an (E)-2-nonenyl group), a decenyl group, an undecenyl group, a dodecenyl group, a dodecadienyl group, a tridecenyl group (preferably a (Z)-tridec-8-enyl group), a tetradecenyl group (preferably a tetradec-9-enyl group), a pentadecenyl group (preferably a (Z)-pentadec-8-enyl group), a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8,11-dienyl group), an octadecenyl group (preferably a (Z)-octadec-9-enyl group), an octadecadienyl group (preferably a (9Z,12Z)-octadeca-9,12-dienyl group), and the like. The alkynyl group having 2 to 18 carbon atoms may be linear or branched or may be chainlike or cyclic. The number of carbon atoms in the alkynyl group is preferably 3 to 18, and more preferably 5 to 18. Specific examples thereof include a propargyl group, a butynyl group, a pentynyl group, a hexynyl group, a heptynyl group, an octynyl group, a nonynyl group, a decynyl group, an undecynyl group, a dodecynyl group, a tetradecynyl group, a pentadecynyl group, a hexadecynyl group, a heptadecynyl group, an octadecynyl group, and the like.

When X represents —NR¹—, R¹ preferably represents a hydrocarbon group having 6 to 24 carbon atoms or a group represented by R²¹-L¹-R²²—. When X represents R²¹-L¹-R²²—, it is preferable that one of R² and R³ represent a hydrogen atom and the other represent a hydrocarbon group having 6 to 24 carbon atoms or a group represented by R³¹-L²-R³²—.

When X represents X represents —O—, it is preferable that R² and R³ each independently represent a hydrocarbon group having 6 to 24 carbon atoms or a group represented by R³¹-L²-R³²—.

It is preferable that R⁴, R⁶, R⁹, R¹⁰, R¹¹, and R¹² each represent a hydrogen atom.

R⁵ is preferably a hydrogen atom, an alkyl group having 1 to 18 carbon atoms, an alkyl group having 1 to 18 carbon atoms substituted with —O(CO)—R⁴² or —(CO)O—R⁴³, an alkyl group having 1 to 18 carbon atoms substituted with an aryl group, or an alkyl group having 1 to 18 carbon atoms substituted with a hydroxyl group. Particularly, R⁵ is preferably an alkyl group having 1 to 18 carbon atoms, an alkyl group having 1 to 18 carbon atoms which may be substituted with —O(CO)—R⁴² or
—(CO)O—R⁴³, an alkyl group having 1 to 12 carbon atoms which may be substituted with an aryl group, or an alkyl group having 1 to 8 carbon atoms which may be substituted with a hydroxyl group, and more preferably an alkyl group having 1 to 18 carbon atoms or an alkyl group having 1 to 18 carbon atoms which may be substituted with —O(CO)—R⁴² or —(CO)O—R⁴³. Where R⁵ is an alkyl group, R⁵ may be linked to R⁴, R⁶, R¹⁰, or R¹² to form a ring which may contain an oxygen atom.

R⁷ and R⁸ preferably each independently represent a hydrogen atom, a hydrocarbon group having 1 to 18 carbon atoms, an alkyl group having 1 to 18 carbon atoms optionally substituted with
—O(CO) R⁴² or —(CO)O—R⁴³, an alkyl group having 1 to 8 carbon atoms which may be substituted with an aryl group, or an alkyl group having 1 to 8 carbon atoms which may be substituted with a hydroxyl group. Alternatively, it is preferable that R⁷ and R⁸ be linked to each other to form a 4- to 7-membered ring of carbon and nitrogen atoms which may contain an oxygen atom.

R⁵ is not linked to R⁷ or R⁸ and does not form a ring with R⁷ or R⁸.

The sum of a+b is preferably 1 or 2, and more preferably 1. The sum of c+d is preferably 1 or 2, and more preferably 1.

Lipids represented by formula (I) and a method for producing the same are described in US2021/0085604 and US2022/0273817, the contents of which are incorporated herein by reference in their entirety.

The compound represented by formula (I) is preferably a compound represented by formula (III) or a pharmaceutically acceptable salt thereof:

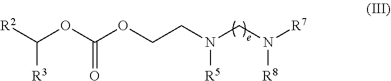

wherein
R² and R³ are each independently a hydrogen atom, a hydrocarbon group having 3 to 24 carbon atoms, or R³¹-L²-R³²—;
R³¹ is a hydrocarbon group having 1 to 24 carbon atoms;
L² is —O(CO)O—, —O(CO)—, —(CO)O—, —O—, or

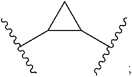 [SMILES: *C1CC1*]

R³² is a divalent hydrocarbon linking group having 1 to 18 carbon atoms;
R⁵ is a hydrogen atom or an alkyl group having 1 to 18 carbon atoms optionally substituted with R³³;
R⁷ and R⁸ are each independently a hydrogen atom or an alkyl group having 1 to 18 carbon atoms optionally substituted with R³³,
R³³ is a hydroxyl group, a carboxyl group, —NR⁴⁵R⁴⁶, —O(CO)O—R⁴¹, —O(CO)—R⁴²,
(CO)O—R⁴³, —O—R⁴⁴, or an aryl or heteroaryl group optionally substituted with R³⁴,
R³⁴ is an alkyl group having 1 to 18 carbon atoms, a hydroxyl group, a carboxyl group, —NR⁴⁵R⁴⁶, —O(CO)O—R⁴¹, —O(CO)—R⁴², —(CO)O—R⁴³, or —O—R⁴⁴,
R⁴¹, R⁴², R⁴³, R⁴⁴, R⁴⁵, and R⁴⁶ are each independently a hydrocarbon group having 1 to 18 carbon atoms; and
e is an integer selected from 2 or 3.

Examples of salts with basic groups include salts with mineral acids such as hydrochloric acid, hydrobromic acid, nitric acid, and sulfuric acid; salts with organic carboxylic acids such as formic acid, acetic acid, citric acid, oxalic acid, fumaric acid, maleic acid, succinic acid, malic acid, tartaric acid, aspartic acid, trichloroacetic acid, and trifluoroacetic acid; and salts with sulfonic acids such as methanesulfonic acid, benzenesulfonic acid, p-toluenesulfonic acid, mesitylenesulfonic acid, and naphthalenesulfonic acid.

Examples of salts with acidic groups include salts with alkali metals such as sodium and potassium; salts with alkaline earth metals such as calcium and magnesium; ammonium salts; salts with nitrogen-containing organic bases such as trimethylamine, triethylamine, tributylamine, pyridine, N,N-dimethylaniline, N-methylpiperidine, N-methylmorpholine, diethylamine, dicyclohexylamine, procaine, dibenzylamine, N-benzyl-β-phenethylamine, 1-ephenamine, and N,N′-dibenzylethylenediamine, and the like.

Among the above salts, pharmacologically acceptable salts are preferred.

Lipids represented by formula (III) and a method for producing the same are described in US2021/0085604 and US2022/0273817. The entirety of each publication is hereby incorporated by reference.

Example lipids represented by formula (III) include:

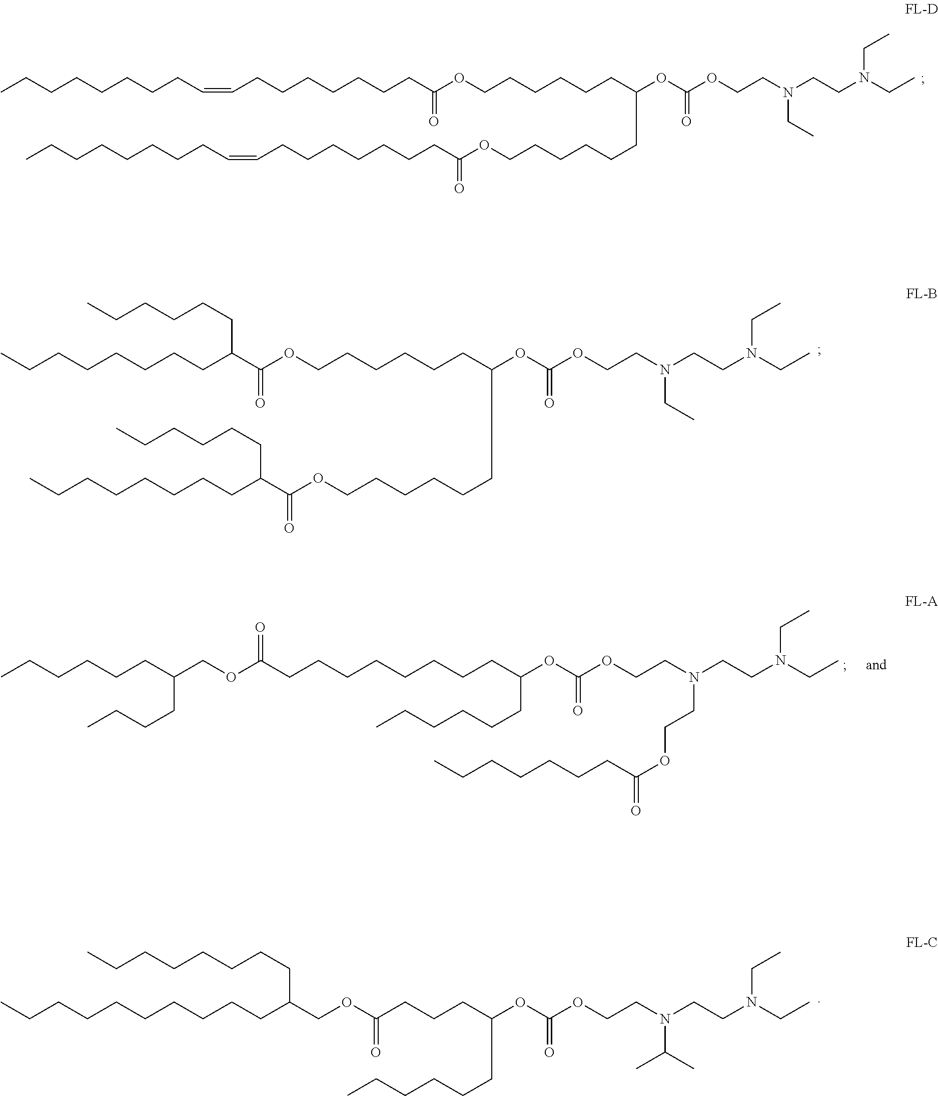

In some embodiments, the ionizable lipid suitable for use in the present disclosure are ionizable lipids of formula (II) or a pharmaceutically acceptable salt thereof:

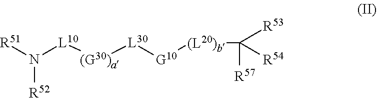

wherein
R⁵¹ and R⁵² are each independently a hydrocarbon group having 1 to 21 carbon atoms optionally substituted with R³⁵;
R³⁵ is a hydroxyl group, -G²⁰-CH(R⁵⁵)(R⁵⁶), —N(R⁵⁸)(R⁵⁹), or -G²⁰-R⁶⁰;
G²⁰ is —(CO)O— or —O(CO)—;
R⁵⁵ and R⁵⁶ are each independently hydrogen or a hydrocarbon group having 1 to 18 carbon atoms;
R⁵⁸ and R⁵⁹ are each independently hydrogen or a cyclic hydrocarbon group having 3 to 6 carbon atoms, optionally substituted with R³⁶;
R⁶⁰ is a hydrocarbon group having 1 to 18 carbon atoms;
R³⁶ is —N(R⁶¹)(R⁶²), or -G²⁰-R⁶⁵;
R⁶¹ and R⁶² are each independently hydrogen or a cyclic hydrocarbon group having 3 to 6 carbon atoms;
R⁶⁵ is a hydrocarbon group having 1 to 18 carbon atoms, -L⁴⁰-CH(R⁶⁶)(R⁶⁷), or -G²⁰-R⁶⁶.
L⁴⁰ is a divalent hydrocarbon group containing 1 to 6 carbon atoms;
R⁶⁶ and R⁶⁷ are each independently a hydrocarbon group containing 1 to 10 carbon atoms or an alkoxy group containing 1 to 10 carbon atoms;
L¹⁰ is a divalent hydrocarbon group containing 1 to 18 carbon atoms;
G³⁰ is —S(CO)N(R⁶⁴);
R⁶⁴ is -L³⁰-G²⁰-CH(R⁵⁵)(R⁵⁶);
a′ is 0 or 1;
G¹⁰ is G²⁰, —O(CO)O—, or —N(R⁶³)C(O);
R⁶³ is a hydrocarbon group containing 1 to 18 carbon atoms;
L²⁰ is a divalent hydrocarbon group containing 1 to 6 carbon atoms;
b′ is 0 or 1;
R⁵³, R⁵⁴, and R⁵⁷ are each independently hydrogen or a hydrocarbon group containing 1 to 18 carbon atoms optionally substituted with R³⁶, and
L³⁰ is a single bond or a hydrocarbon group containing 1 to 18 carbon atoms.

The compound represented by formula (II) may be represented by formula (IIa)

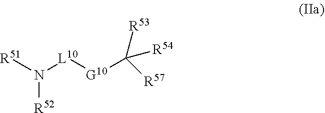

wherein
R⁵¹ and R⁵² are each independently a hydrocarbon group having 1 to 21 carbon atoms optionally substituted with R³⁵;
R³⁵ is a hydroxyl group or -G²⁰-CH(R⁵⁵)(R⁵⁶);
G²⁰ is —(CO)O— or —O(CO)—;
R⁵⁵ and R⁵⁶ are each independently hydrogen or a hydrocarbon group having 1 to 18 carbon atoms;
L¹⁰ is a divalent hydrocarbon group containing 1 to 18 carbon atoms;
G¹⁰ is —(CO)O— or —O(CO)—; and
R⁵³, R⁵⁴, and R⁵⁷ are each independently hydrogen or a hydrocarbon group containing 1 to 18 carbon atoms.

The compound represented by formula (II) may be represented by formula (IIb)

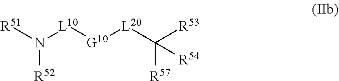

wherein
R⁵¹ and R⁵² are each independently a hydrocarbon group having 1 to 21 carbon atoms;
L¹⁰ is a divalent hydrocarbon group containing 1 to 18 carbon atoms;
G¹⁰ is —O(CO)O—;
L²⁰ is a divalent hydrocarbon group containing 1 to 6 carbon atoms;
R⁵³, R⁵⁴, and R⁵⁷ are each independently hydrogen or a hydrocarbon group containing 1 to 18 carbon atoms optionally substituted with R³⁶;
R³⁶ is —O(CO)—R⁶⁵;
R⁶⁵ is a hydrocarbon group having 1 to 18 carbon atoms or -L⁴⁰-CH(R⁶⁶)(R⁶⁷);
L⁴⁰ is a divalent hydrocarbon group containing 1 to 6 carbon atoms; and
R⁶⁶ and R⁶⁷ are each independently an alkoxy group containing 1 to 10 carbon atoms.

The compound represented by formula (II) may be represented by formula (IIc)

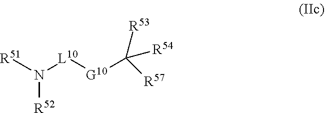

wherein
R⁵¹ and R⁵² are each independently a hydrocarbon group having 1 to 21 carbon atoms;
L¹⁰ is a divalent hydrocarbon group containing 1 to 18 carbon atoms;
G¹⁰ is —N(R⁶³)C(O)—;
R⁶³ is a hydrocarbon group containing 1 to 18 carbon atoms;
R⁵³, R⁵⁴, and R⁵⁷ are each independently hydrogen or a hydrocarbon group containing 1 to 18 carbon atoms optionally substituted with R³⁶;
R³⁶ is —(CO)OR⁶⁵;
R⁶⁵ is -L⁴⁰-CH(R⁶⁶)(R⁶⁷);
L⁴⁰ is a divalent hydrocarbon group containing 1 to 6 carbon atoms; and
R⁶⁶ and R⁶⁷ are each independently a hydrocarbon group containing 1 to 10 carbon atoms.

The compound represented by formula (II) may be represented by formula (IId)

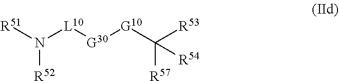

wherein
R⁵¹ and R⁵² are each independently a hydrocarbon group having 1 to 21 carbon atoms;
L¹⁰ is a divalent hydrocarbon group containing 1 to 18 carbon atoms;
G³⁰ is —S(CO)NR⁶⁴—;
R⁶⁴ is -L³⁰-G²⁰-CH(R⁵⁵)(R⁵⁶)
L³⁰ is a single bond or a hydrocarbon group containing 1 to 18 carbon atoms;
G²⁰ is —(CO)O—;
R⁵⁵ and R⁵⁶ are each independently hydrogen or a hydrocarbon group having 1 to 18 carbon atoms;
G¹⁰ is —(CO)O—; and
R⁵³, R⁵⁴, and R⁵⁷ are each independently hydrogen or a hydrocarbon group containing 1 to 18 carbon atoms.

In some embodiments a compound of formula (II), (IIa), (IIb), (IIc), or (IId) may be in the form of a salt.

In some embodiments, the ionizable lipid suitable for use in the present disclosure are ionizable lipids of formula (VII) or a pharmaceutically acceptable salt thereof:

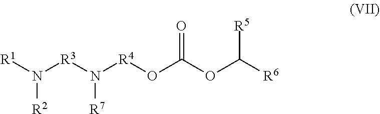

wherein
R¹ and R² are each independently a hydrocarbon group having 1 to 18 carbon atoms, and R³ is a hydrocarbon group having 2 to 8 carbon atoms, wherein the hydrocarbon groups represented by R¹, R², and R³ are each independently optionally substituted with one or more substituents selected from —OH, COOH, —NR⁵¹R⁵², —OC(O)O—R⁵³, —C(O)O—R⁵⁴, —OC(O)—R⁵⁵, and —O—R⁵⁶;
R⁴ is a hydrocarbon group having 1 to 8 carbon atoms;
R⁵ and R⁶ are each independently a hydrocarbon group having 1 to 8 carbon atoms or —R⁸-L¹-R⁹, excluding a case where both R⁵ and R⁶ are hydrocarbon groups having 1 to 8 carbon atoms;
R⁷ is —R¹⁰-L²-R¹¹-L³-R¹²,
R⁵¹ and R⁵² are each independently a hydrocarbon group having 1 to 8 carbon atoms;
R⁵³, R⁵⁴, R⁵⁵, and R⁵⁶ are each independently a hydrocarbon group having 1 to 24 carbon atoms;
the hydrocarbon groups represented by R⁵³, R⁵⁴, R⁵⁵, and R⁵⁶ are each independently optionally substituted with an aryl group having 6 to 20 carbon atoms or —S—R⁵⁸, wherein the aryl group having 6 to 20 carbon atoms is optionally substituted with —OH, COOH, —NR⁵¹R⁵², —OC(O)O—R⁵³, —C(O)O—R⁵⁴, —OC(O)—R⁵⁵, —O—R⁵⁶, or —R⁵⁹—R⁵⁷, wherein
R⁵⁸ and R⁵⁹ are each independently a hydrocarbon group having 1 to 12 carbon atoms;
R⁵⁷ is —OH, COOH, —NR⁶¹R⁶², —OC(O)O—R⁶³, —C(O)O—R⁶⁴, —OC(O)—R⁶⁵, or —O—R⁶⁶;
R⁶¹ and R⁶² are each independently a hydrocarbon group having 1 to 8 carbon atoms;
R⁶³, R⁶⁴, R⁶⁵, and R⁶⁶ are each independently a hydrocarbon group having 1 to 24 carbon atoms;
the hydrocarbon groups represented by R⁶³, R⁶⁴, R⁶⁵, and R⁶⁶ are each independently optionally substituted with an aryl group having 6 to 20 carbon atoms or —S—R⁶⁸ wherein the aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or —R⁶⁹—R⁶⁷,
R⁶⁸ and R⁶⁹ are each independently a hydrocarbon group having 1 to 12 carbon atoms;
R⁶⁷ is —OH, COOH, —NR⁶¹R⁶², —OC(O)O—R⁶³, —C(O)O—R⁶⁴, —OC(O)—R⁶⁵, or —O—R⁶⁶,
L¹, L², and L³ are each independently —OC(O)O—, —C(O)O—, —OC(O)—, or —O—;
R⁸ is a hydrocarbon group having 1 to 12 carbon atoms;
R⁹ is a hydrocarbon group having 1 to 24 carbon atoms;
R¹⁰ is a hydrocarbon group having 1 to 8 carbon atoms;
R¹¹ is a hydrocarbon group having 1 to 24 carbon atoms;
R¹² is a hydrocarbon group having 1 to 24 carbon atoms;
the hydrocarbon groups represented by R⁹ and R¹² are each independently optionally substituted with an aryl group, —OC(O)O—R⁵³, —C(O)O—R⁵⁴, —OC(O)—R⁵⁵, or —S—R⁵⁸; and
the hydrocarbon groups represented by R¹¹ are each independently optionally substituted with —OC(O)O—R⁵³, —C(O)O—R⁵⁴, or —OC(O)—R⁵⁵.

A hydrocarbon group having 1 to 24 carbon atoms, a hydrocarbon group having 1 to 18 carbon atoms, a hydrocarbon group having 1 to 12 carbon atoms, a hydrocarbon group having 2 to 8 carbon atoms, and a hydrocarbon group having 1 to 8 carbon atoms are each preferably an alkyl group, an alkenyl group, or an alkynyl group.

The alkyl group may be linear or branched, or may be chainlike or cyclic. Specifically, examples of the alkyl group include a methyl group, an ethyl group, a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, a tridecyl group, a trimethyldodecyl group (preferably a 3,7, 11-trimethyldodecyl group), a tetradecyl group, a pentadecyl group, a hexadecyl group, a tetramethylhexadecyl group (preferably a 3,7,11, 15-tetramethylhexadecyl group), a heptadecyl group, an octadecyl group, a 2-butylhexyl group, a 2-butyloctyl group, a 1-pentylhexyl group, a 2-pentylheptyl group, a 3-pentyloctyl group, a 1-hexylheptyl group, a 1-hexylnonyl group, a 2-hexyloctyl group, a 2-hexyldecyl group, a 3-hexylnonyl group, a 1-heptyloctyl group, a 2-heptylnonyl group, a 2-heptylundecyl group, a 3-heptyldecyl group, a 1-octylnonyl group, a 2-octyldecyl group, a 2-octyldodecyl group, a 3-octylundecyl group, a 2-nonylundecyl group, a 3-nonyldodecyl group, a 2-decyldodecyl group, a 2-decyltetradecyl group, a 3-decyltridecyl group, a 2-(4,4-dimethylpentan-2-yl)-5,7,7-trimethyloctyl group, and the like.

The alkenyl group may be linear or branched, or may be chainlike or cyclic. Specifically, examples of the alkenyl group include an allyl group, a prenyl group, a pentenyl group, a hexenyl group, a heptenyl group, an octenyl group, a nonenyl group (preferably a (Z)-2-nonenyl group or an (E)-2-nonenyl group), a decenyl group, an undecenyl group, a dodecenyl group, a dodecadienyl group, a tridecenyl group (preferably a (Z)-tridec-8-enyl group), a tetradecenyl group (preferably a tetradec-9-enyl group), a pentadecenyl group (preferably a (Z)-pentadec-8-enyl group), a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8,11-dienyl group), an octadecenyl group (preferably a (Z)-octadec-9-enyl group), an octadecadienyl group (preferably a (9Z,12Z)-octadeca-9, 12-dienyl group), and the like. The alkenyl groups of the foregoing preferably have one double bond or two double bonds.

The alkynyl group may be linear or branched, or may be chainlike or cyclic. Specifically, examples of alkynyl group include a propargyl group, a butynyl group, a pentynyl group, a hexynyl group, a heptynyl group, an octynyl group, a nonynyl group, a decynyl group, an undecynyl group, a dodecynyl group, a tetradecynyl group, a pentadecynyl group, a hexadecynyl group, a heptadecynyl group, an octadecynyl group, and the like. The alkynyl groups of the foregoing preferably have one triple bond or two triple bonds.

R⁶⁹ is preferably an alkylene group having 1 to 12 carbon atoms or an alkenylene group having 2 to 12 carbon atoms. The alkylene group having 1 to 12 carbon atoms and the alkenylene group having 2 to 12 carbon atoms may be linear or branched, or may be chainlike or cyclic. Specifically, examples thereof include a methylene group, an ethylene group, a trimethylene group, a tetramethylene group, a pentamethylene group, a hexamethylene group, a heptamethylene group, an octamethylene group, a nonamethylene group, a decamethylene group, an undecamethylene group, and the like.

The aryl group preferably has 6 to 20 carbon atoms, more preferably has 6 to 18 carbon atoms, and even more preferably 6 to 10 carbon atoms. Specifically, examples of the aryl group include a phenyl group, a naphthyl group, an anthracenyl group, a phenanthrenyl group, and the like.

R¹ and R² are each independently preferably a hydrocarbon group having 1 to 12 carbon atoms, more preferably a hydrocarbon group having 1 to 6 carbon atoms, and even more preferably a hydrocarbon group having 1 to 3 carbon atoms.

R³ is preferably a hydrocarbon group having 2 to 6 carbon atoms and more preferably a hydrocarbon group having 2 to 4 carbon atoms.

The hydrocarbon groups represented by R¹, R², and R³ are each independently optionally substituted with preferably —OH.

L¹ and L³ are each independently preferably —C(O)O— or —OC(O)—.

L² is preferably —OC(O)O—, —C(O)O—, or —OC(O)—.

R⁸ is preferably a hydrocarbon group having 1 to 10 carbon atoms and more preferably a hydrocarbon group having 1 to 8 carbon atoms.

R⁹ is preferably a hydrocarbon group having 1 to 20 carbon atoms and more preferably a hydrocarbon group having 1 to 16 carbon atoms.

R¹¹ is preferably a hydrocarbon group having 1 to 16 carbon atoms and more preferably a hydrocarbon group having 1 to 9 carbon atoms.

R¹² is preferably a hydrocarbon group having 1 to 20 carbon atoms and more preferably a hydrocarbon group having 1 to 16 carbon atoms.

The hydrocarbon groups represented by R⁹ and R¹² are each independently optionally substituted with preferably an aryl group or —S—R⁵⁸, wherein R⁵⁸ is preferably a hydrocarbon group having 1 to 8 carbon atoms.

The hydrocarbon group represented by R¹¹ is optionally substituted with preferably —C(O)O—R⁵⁵ or —OC(O)—R⁵⁶, where R⁵⁵ and R⁵⁶ are each independently a hydrocarbon group having 1 to 16 carbon atoms.

The hydrocarbon groups represented by R⁵⁵ and R⁵⁶ are each independently optionally substituted with preferably an aryl group having 6 to 20 carbon atoms or —S—R⁵⁸.

In some embodiments, the ionizable lipid suitable for use in the present disclosure are ionizable lipids of formula (VIII) or a pharmaceutically acceptable salt thereof:

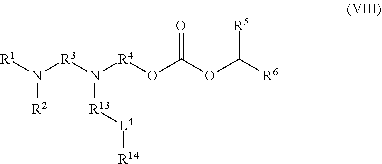

wherein
R¹ and R² are each independently a hydrocarbon group having 1 to 18 carbon atoms, and R³ is a hydrocarbon group having 2 to 8 carbon atoms, wherein the hydrocarbon groups represented by R¹, R², and R³ are each independently optionally substituted with —OH, COOH, —NR⁵¹R⁵², —OC(O)O—R⁵³, —C(O)O—R⁵⁴, —OC(O)—R⁵⁵, or —O—R⁵⁶;
R⁴ is a hydrocarbon group having 1 to 8 carbon atoms;
R⁵ and R⁶ are each independently a hydrocarbon group having 1 to 8 carbon atoms or —R⁸-L¹-R⁹, excluding a case where both R⁵ and R⁶ are hydrocarbon groups having 1 to 8 carbon atoms;
L¹ and L⁴ are each independently —OC(O)O—, —C(O)O—, —OC(O)—, or —O—;
R⁸ is a hydrocarbon group having 1 to 12 carbon atoms;
R⁹ is a hydrocarbon group having 1 to 24 carbon atoms, wherein the hydrocarbon group represented by R⁹ is optionally substituted with an aryl group, —OC(O)O—R⁵³, —C(O)O—R⁵⁴, —OC(O)—R⁵⁵, or —S—R⁵⁸,
R⁵¹ and R⁵² are each independently a hydrocarbon group having 1 to 8 carbon atoms;
R²¹, R²³, R²⁵, R⁵³, R⁵⁴, R⁵⁵, and R⁵⁶ are each independently a hydrocarbon group having 1 to 24 carbon atoms;
the hydrocarbon groups represented by R⁵³, R⁵⁴, R⁵⁵, and R⁵⁶ are each independently optionally substituted with an aryl group having 6 to 20 carbon atoms or —S—R⁵⁸, wherein the aryl group having 6 to 20 carbon atoms is optionally substituted with —OH, COOH, —NR⁵¹R⁵², —OC(O)O—R⁵³, —C(O)O—R⁵⁴, —OC(O)—R⁵⁵, —O—R⁵⁶, or —R⁵⁹—R⁵⁷,
R⁵⁸ and R⁵⁹ are each independently a hydrocarbon group having 1 to 12 carbon atoms;
R⁵⁷ is —OH, COOH, —NR⁵¹R⁵², —OC(O)O—R⁵³, —C(O)O—R⁵⁴, —OC(O)—R⁵⁵, or —O—R⁵⁶,
R¹³ is a hydrocarbon group having 1 to 8 carbon atoms;
R¹⁴ is —R¹⁵-L⁵-R¹⁶, wherein R¹⁵ is a hydrocarbon group having 1 to 24 carbon atoms, L⁵ is —OC(O)O—, —C(O)O—, —OC(O)—, or —O—, and R¹⁶ is a hydrocarbon group having 1 to 24 carbon atoms;
the hydrocarbon group having 1 to 24 carbon atoms represented by R¹⁵ is optionally substituted with —OC(O)O—R⁵³, —C(O)O—R⁵⁴, or —OC(O)—R⁵⁵; and
the hydrocarbon group having 1 to 24 carbon atoms represented by R¹⁶ is optionally substituted with an aryl group having 6 to 20 carbon atoms, —OC(O)O—R⁵³, —C(O)O—R⁵⁴, —OC(O)—R⁵⁵ or —S—R⁵⁸.

In Formula (VIII), R¹ and R² are each independently preferably a hydrocarbon group having 1 to 12 carbon atoms, more preferably a hydrocarbon group having 1 to 6 carbon atoms, and even more preferably a hydrocarbon group having 1 to 3 carbon atoms.

R³ is preferably a hydrocarbon group having 2 to 6 carbon atoms and more preferably a hydrocarbon group having 2 to 4 carbon atoms.

The hydrocarbon groups represented by R¹, R², and R³ are each independently optionally substituted with preferably-OH.

L¹ is preferably —C(O)O— or —OC(O)—.

R⁸ is preferably a hydrocarbon group having 1 to 10 carbon atoms and more preferably a hydrocarbon group having 1 to 8 carbon atoms.

R⁹ is preferably a hydrocarbon group having 1 to 18 carbon atoms, and the hydrocarbon group represented by R⁹ is optionally substituted with an aryl group having 6 to 20 carbon atoms or —S—R⁵⁸.

R¹⁴ is preferably —R¹⁵-L⁵-R¹⁶, where R¹⁵ is a hydrocarbon group having 1 to 18 carbon atoms, L⁵ is —OC(O)O—, and R¹⁶ is a hydrocarbon group having 1 to 18 carbon atoms.

The hydrocarbon group having 1 to 18 carbon atoms represented by R¹⁵ is optionally substituted with preferably —C(O)O—R⁵⁵ or —OC(O)—R⁵⁶. R⁵⁵ and R⁵⁶ are each independently a hydrocarbon group having 1 to 16 carbon atoms, and the hydrocarbon groups represented by R⁵⁵ and R⁵⁶ are each independently optionally substituted with an aryl group having 6 to 20 carbon atoms or —S—R⁵⁸.

The hydrocarbon group having 1 to 18 carbon atoms represented by R¹⁶ is optionally substituted with preferably an aryl group or —S—R⁵⁸.

In some embodiments, the ionizable lipid suitable for use in the present disclosure are ionizable lipids of formula (IX) or a pharmaceutically acceptable salt thereof:

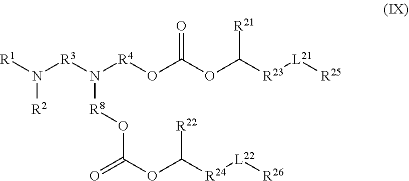

wherein
R¹ and R² are each independently a hydrocarbon group having 1 to 18 carbon atoms, and R³ is a hydrocarbon group having 2 to 8 carbon atoms, wherein the hydrocarbon groups represented by R¹, R², and R³ are each optionally substituted with —OH, COOH, —NR⁵¹R⁵², —OC(O)O—R⁵³, —C(O)O—R⁵⁴, —OC(O)—R⁵⁵, or —O—R⁵⁶,
R⁴ and R⁸ are each independently a hydrocarbon having 1 to 8 carbon atoms;
R²¹ and R²² are each independently a hydrocarbon group having 1 to 18 carbon atoms;
R²³ and R²⁴ are each independently a hydrocarbon group having 1 to 12 carbon atoms;
R²⁵ and R²⁶ are each independently a hydrocarbon group having 1 to 24 carbon atoms;
L²¹ and L²² are each independently —OC(O)O—, —C(O)O—, —OC(O)—, or —O—;
the hydrocarbon groups represented by R²⁵ and R²⁶ are each independently optionally substituted with an aryl group having 6 to 20 carbon atoms, —OC(O)O—R⁵³, —C(O)O—R⁵⁴, —OC(O)—R⁵⁵, or —S—R⁵⁸, wherein the aryl group having 6 to 20 carbon atoms is optionally substituted with OH, COOH, —NR⁵¹R⁵², —OC(O)O—R⁵³, —C(O)O—R⁵⁴, —OC(O)—R⁵⁵, —O—R⁵⁶, or —R⁵⁹—R⁵⁷, and
R⁵¹ and R⁵² are each independently a hydrocarbon group having 1 to 8 carbon atoms;
R⁵³, R⁵⁴, R⁵⁵, and R⁵⁶ are each independently a hydrocarbon group having 1 to 18 carbon atoms;
R⁵⁷ is —OH, COOH, —NR⁵¹R⁵², —OC(O)O—R⁵³, —C(O)O—R⁵⁴, —OC(O)—R⁵⁵, or —O—R⁵⁶; and

R⁵⁸ and R⁵⁹ are each independently a hydrocarbon group having 1 to 12 carbon atoms. In Formula (IX), R¹ and R² are each independently preferably a hydrocarbon group having 1 to 12 carbon atoms, more preferably a hydrocarbon group having 1 to 6 carbon atoms, and even more preferably a hydrocarbon group having 1 to 3 carbon atoms. The hydrocarbon groups represented by R¹ and R² are each independently optionally substituted with preferably-OH, and more preferably a hydrocarbon having no substituent.

R³ is preferably a hydrocarbon group having 2 to 6 carbon atoms and more preferably a hydrocarbon group having 2 to 4 carbon atoms.

R²¹ and R²² are each independently preferably a hydrocarbon group having 1 to 12 carbon atoms, more preferably a hydrocarbon group having 1 to 8 carbon atoms, and even more preferably a hydrocarbon group having 1 to 6 carbon atoms.

R²³ and R²⁴ are each independently preferably a hydrocarbon group having 1 to 10 carbon atoms and more preferably a hydrocarbon group having 1 to 8 carbon atoms.

R²⁵ and R²⁶ are each independently preferably a hydrocarbon group having 1 to 20 carbon atoms, more preferably a hydrocarbon group having 1 to 16 carbon atoms, and even more preferably a hydrocarbon group having 1 to 12 carbon atoms.

L²¹ and L²² are each independently preferably —C(O)O— or —OC(O)—.

In some embodiments, the ionizable lipid suitable for use in the present disclosure are ionizable lipids of formula (X) or a pharmaceutically acceptable salt thereof:

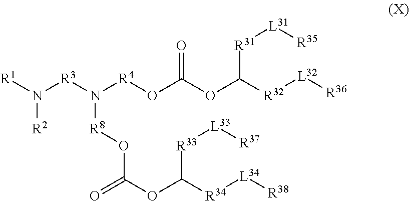

wherein
R¹ and R² are each independently a hydrocarbon group having 1 to 18 carbon atoms, and R³ is a hydrocarbon group having 2 to 8 carbon atoms, wherein the hydrocarbon groups represented by R¹, R², and R³ are each independently optionally substituted with —OH, COOH, —NR⁵¹R⁵², —OC(O)O—R⁵³, —C(O)O—R⁵⁴, —OC(O)—R⁵⁵, or —O—R⁵⁶;
R⁴ and R⁸ are each independently a hydrocarbon group having 1 to 8 carbon atoms;
R³¹, R³², R³³, and R³⁴ are each independently a hydrocarbon group having 1 to 12 carbon atoms,
R³⁵, R³⁶, R³⁷, and R³⁸ are each independently a hydrocarbon group having 1 to 24 carbon atoms;
L³¹, L³², L³³, and L³⁴ are each independently —OC(O)O—, —C(O)O—, —OC(O)—, or —O—;
the hydrocarbon groups represented by R³⁵, R³⁶, R³⁷, and R³⁸ are each independently optionally substituted with an aryl group having 6 to 20 carbon atoms, —OC(O)O—R⁵³, —C(O)O—R⁵⁴, —OC(O)—R⁵⁵, or S—R⁵⁸, wherein the aryl group having 6 to 20 carbon atoms is optionally substituted with OH, COOH, —NR⁵¹R⁵², —OC(O)O—R⁵³, —C(O)O—R⁵⁴, —OC(O)—R⁵⁵, —O—R⁵⁶, or —R⁵⁹—R⁵⁷, and
R⁵¹ and R⁵² are each independently a hydrocarbon group having 1 to 8 carbon atoms;
R⁵³, R⁵⁴, R⁵⁵, and R⁵⁶ are each independently a hydrocarbon group having 1 to 18 carbon atoms;
R⁵⁷ is —OH, COOH, —NR⁵¹R⁵², —OC(O)O—R⁵³, —C(O)O—R⁵⁴, —OC(O)—R⁵⁵, or —O—R⁵⁶; and
R⁵⁸ and R⁵⁹ are each independently a hydrocarbon group having 1 to 12 carbon atoms.

In Formula (X), R¹ and R² are each independently preferably a hydrocarbon group having 1 to 12 carbon atoms, more preferably a hydrocarbon group having 1 to 6 carbon atoms, and even more preferably a hydrocarbon group having 1 to 3 carbon atoms. The hydrocarbon groups represented by R¹ and R² are each independently optionally substituted with preferably —OH, and more preferably a hydrocarbon having no substituent.

R³ is preferably a hydrocarbon group having 2 to 6 carbon atoms and more preferably a hydrocarbon group having 2 to 4 carbon atoms.

R³¹, R³², R³³, and R³⁴ are each independently preferably a hydrocarbon group having 1 to 10 carbon atoms, more preferably a hydrocarbon group having 1 to 8 carbon atoms, and even more preferably a hydrocarbon group having 1 to 3 carbon atoms.

R³⁵, R³⁶, R³⁷, and R³⁸ are each independently preferably a hydrocarbon group having 1 to 20 carbon atoms, more preferably a hydrocarbon group having 1 to 16 carbon atoms, and even more preferably a hydrocarbon group having 1 to 12 carbon atoms. The hydrocarbon groups represented by R³⁵, R³⁶, R³⁷, and R³⁸ are each independently substituted with preferably an aryl group having 6 to 20 carbon atoms or S—R⁵⁸, and more preferably —S—R⁵⁸.

R³⁵, R³⁶, R³⁷, and R³⁸ are each independently preferably a hydrocarbon group having 1 to 12 carbon atoms substituted with —S—R⁵⁸, or a hydrocarbon group having 1 to 12 carbon atoms.

L³¹, L³², L³³, and L³⁴ are each independently preferably —C(O)O—, or —OC(O)—.

R⁵⁸ is preferably a hydrocarbon group having 1 to 10 carbon atoms and more preferably a hydrocarbon group having 1 to 8 carbon atoms.

The compound according to an embodiment of the present invention may form a salt (e.g. a pharmaceutically acceptable salt).

Examples of salts in basic group include salts with mineral acids such as hydrochloric acid, hydrobromic acid, nitric acid, and sulfuric acid; salts with organic carboxylic acids such as formic acid, acetic acid, citric acid, oxalic acid, fumaric acid, maleic acid, succinic acid, malic acid, tartaric acid, aspartic acid, trichloroacetic acid, and trifluoroacetic acid; and salts with sulfonic acids such as methanesulfonic acid, benzenesulfonic acid, p-toluenesulfonic acid, mesitylenesulfonic acid, and naphthalenesulfonic acid.

Examples of salts in acidic group include salts with alkali metals such as sodium and potassium; salts with alkaline earth metals such as calcium and magnesium; ammonium salts; salts with nitrogen-containing organic bases such as trimethylamine, triethylamine, tributylamine, pyridine, N,N-dimethylaniline, N-methylpiperidine, N-methylmorpholine, diethylamine, dicyclohexylamine, procaine, dibenzylamine, N-benzyl-β-phenethylamine, 1-ephenamine, and N,N′-dibenzylethylenediamine; and the like.

Among the above-described salts, for example, pharmacologically acceptable salts are preferable.

The lipid represented by the formula (VII) and a method for producing same are described in WO2022/230964A, the entire teachings of which are incorporated herein by reference.

Example lipids represented by formula (VII) include:

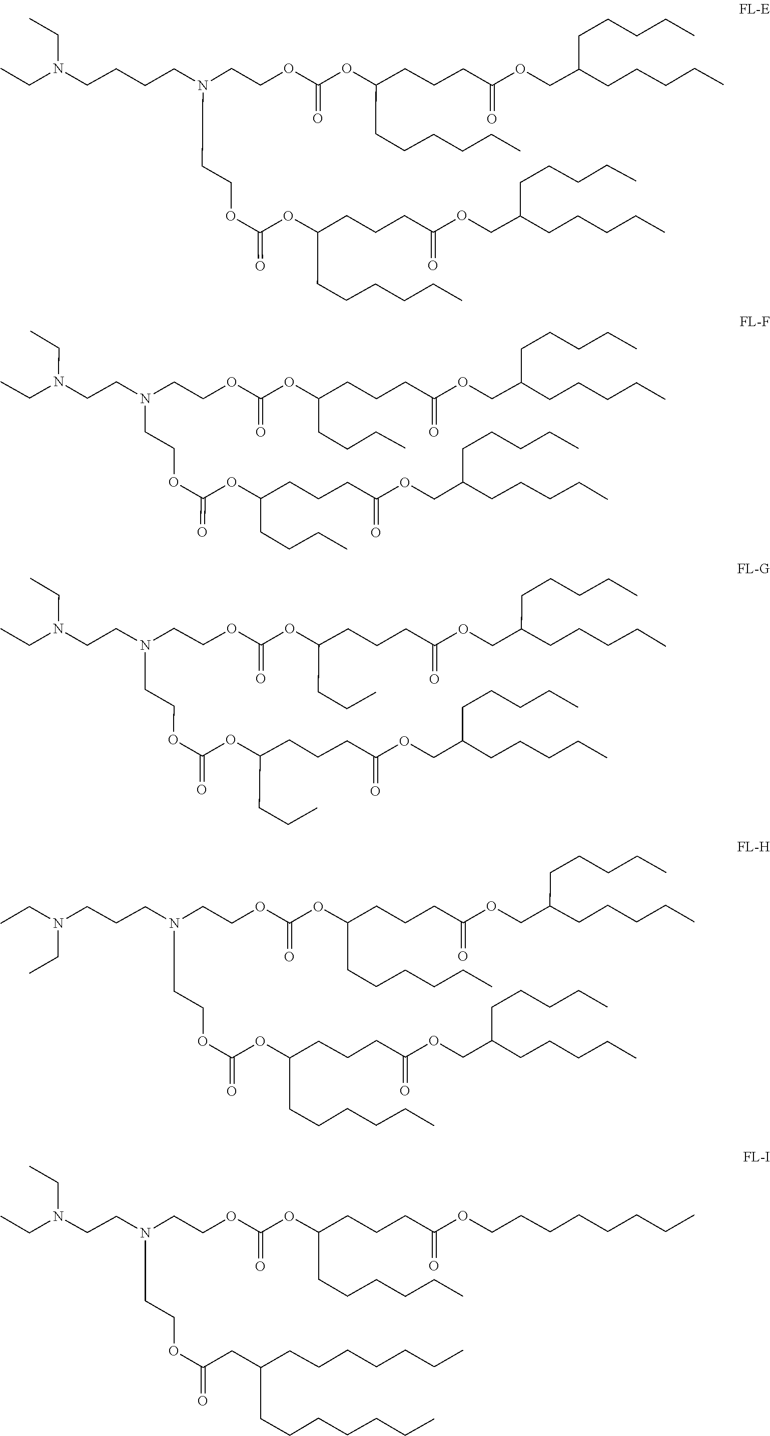

A hydrocarbon group having 1 to 21 carbon atoms from R⁵¹ or R⁵² is preferably an alkyl group having 1 to 21 carbon atoms, an alkenyl group having 2 to 21 carbon atoms, or an alkynyl group having 2 to 21 carbon atoms, more preferably an alkyl group having 1 to 21 carbon atoms, or an alkenyl group having 2 to 21 carbon atoms. The alkyl group having 1 to 21 carbon atoms may be linear or branched, and may be chain or cyclic. The number of carbon atoms is preferably 3 to 21, and more preferably 5 to 21 carbon atoms. Examples include propyl group, isopropyl group, cyclopropyl group, butyl group, isobutyl group, tert-butyl group, cyclobutyl group, pentyl group, cyclopentyl group, hexyl group, cyclohexyl group, heptyl group, octyl group, nonyl group, decyl group, undecyl group, dodecyl group, tridecyl group, trimethyldodecyl group (preferably a 3,7,11-trimethyldodecyl group), tetradecyl group, pentadecyl group, hexadecyl group, heptadecyl group and octadecyl group. The alkenyl group having 2 to 18 carbon atoms may be linear or branched, and may be chain or cyclic. The number of carbon atoms is preferably 3 to 21, and more preferably 5 to 18. Examples include allyl group, prenyl group, pentanyl group, hexenyl group, heptenyl group, octenyl group, nonenyl group (preferably (Z)-2-nonenyl group or (E)-2-nonenyl group), decenyl group, undecenyl group, dodecenyl group, dodecadienyl group, tridecenyl group (preferably (Z)-trideca-8-enyl group), tetradecenyl group (preferably tetradeca-9-enyl group), pentadecenyl group (preferably (Z)-pentadeca-8-enyl group), hexadecenyl group (preferably (Z)-hexadeca-9-enyl group), hexadecadienyl group, heptadecenyl group (preferably (Z)-heptadeca-8-enyl group), heptadecadienyl group (preferably (8Z, 11Z)-heptadeca-8,11-dienyl group), octadecenyl group (preferably (Z)-octadeca-9-enyl group), octadecadienyl groups (preferably (9Z, 12Z)-octadeca-9,12-dienyl group). The alkynyl group having 2 to 21 carbon atoms may be linear or branched, and may be chain or cyclic. The number of carbon atoms is preferably 3 to 21, and more preferably 5 to 21 carbon atoms. Examples include propargyl group, butynyl group, pentynyl group, hexynyl group, heptynyl group, octynyl group, nonynyl group, decynyl group, undecynyl group, dodecynyl group, tetradecynyl group, pentadecynyl group, hexadecynyl group, heptadecynyl group, octadecynyl group and the like. Examples of hydrocarbon groups having 1 to 18 carbon atoms include those example groups specifically listed among the hydrocarbon groups having 1 to 21 carbon atoms that have 1 to 18 carbon atoms.

For a cyclic hydrocarbon group, a cycloalkyl group having 3 to 10 carbon atoms, a cycloalkenyl group having 3 to 10 carbon atoms, a cycloalkynyl group having 3 to 10 carbon atoms, or an aryl group having 6 to 10 carbon atoms are preferable.

For a hydrocarbon group having 1 to 6 carbon atoms an alkyl group having 1 to 6 carbon atoms, an alkenyl group having 2 to 6 carbon atoms or an alkynyl group having 2 to 6 carbon atoms, is preferable, and an alkyl group having 1 to 6 carbon atoms or an alkenyl group having 2 to 6 carbon atoms is more preferrable. The alkyl group having 1 to 6 carbon atoms may be linear or branched, and may be a chain or cyclic. Specific examples thereof include propyl group, isopropyl group, cyclopropyl group, butyl group, isobutyl group, tert-butyl group, cyclobutyl group, pentyl group, cyclopentyl group and hexyl group. The alkenyl group having 2 to 6 carbon atoms may be linear or branched, and may be a chain or cyclic. Specific examples thereof include allyl, prenyl, pentenyl, and hexenyl. The alkynyl group having 2 to 6 carbon atoms may be linear or branched, and may be a chain or cyclic. Specific examples thereof include propargyl, butynyl, pentynyl, and hexynyl.

The hydrocarbon group having 1 to 10 carbon atoms from R⁶⁶ and R⁶⁷ is preferably an alkyl group having 1 to 10 carbon atoms, an alkenyl group having 2 to 10 carbon atoms, or an alkynyl group having 2 to 10 carbon atoms, and preferably an alkyl group having 1 to 10 carbon atoms or an alkenyl group having 2 to 10 carbon atoms. The alkyl group having 1 to 10 carbon atoms may be linear or branched, and may be chain or cyclic. The number of carbon atoms is preferably 3 to 10, and more preferably 5 to 10 carbon atoms. Specific examples include a propyl group, isopropyl group, cyclopropyl group, butyl group, isobutyl group, tert-butyl group, cyclobutyl group, pentyl group, cyclopentyl group, hexyl group, cyclohexyl group, heptyl group, octyl group, nonyl group, and decyl group. The alkenyl group having 2 to 10 carbon atoms may be linear or branched, and may be chain or cyclic. The number of carbon atoms is preferably 3 to 10, more preferably 5 to 10. Specific examples include allyl group, prenyl group, pentenyl group, hexenyl group, heptenyl group, octenyl group, a nonenyl group (preferably (Z)-2-nonenyl group or (E)-2-nonenyl group), and decenyl group. The alkynyl group having 2 to 10 carbon atoms may be linear or branched, and may be chain or cyclic. The number of carbon atoms is preferably 3 to 10, and more preferably 5 to 10 carbon atoms. Specific examples thereof include propargyl group, butynyl group, pentynyl group, hexynyl group, heptynyl group, octynyl group, noninyl group and a decynyl group.

Example ionizable lipids of formula (II) include:
MC3; ([(6Z,9Z,28Z,31Z)-heptatriaconta-6,9,28,31-tetraen-19-yl] 4-(dimethylamino) butanoate) WO2010/054405

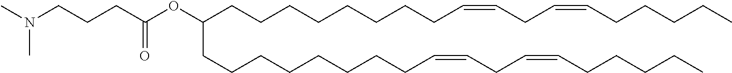 [SMILES: CCCCC/C=C\C/C=C\CCCCCCCCC(CCCCCCCC/C=C\C/C=C\CCCCC)OC(=O)CCCN(C)C]

L-319; (bis[(Z)-non-2-enyl] 9-[4-(dimethylamino)butanoyloxy] heptadecanedioate) WO2011/153493, WO2013/086354, WO2013/086322

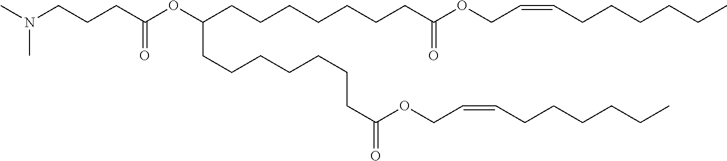 [SMILES: CCCCCC/C=C\COC(=O)CCCCCCCC(CCCCCCCC(=O)OC/C=C\CCCCCC)OC(=O)CCCN(C)C]

ALC-0315; (6-[6-(2-hexyldecanoyloxy)hexyl-(4-hydroxybutyl) amino] hexyl 2-hexyldecanoate) WO2017/075331

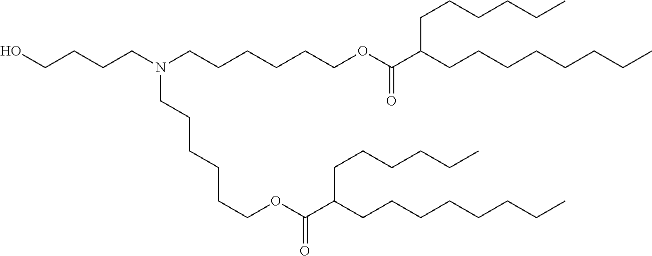 [SMILES: CCCCCCCCC(CCCCCC)C(=O)OCCCCCCN(CCCCO)CCCCCCOC(=O)C(CCCCCC)CCCCCCCC]

SM-102; (heptadecan-9-yl 8-[2-hydroxyethyl-(6-oxo-6-undecoxyhexyl) amino]octanoate) WO2017/099823

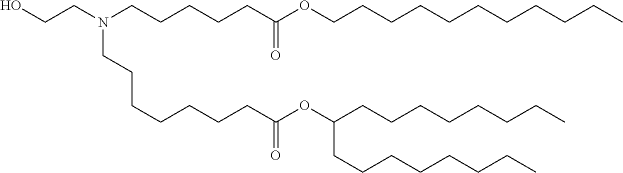 [SMILES: CCCCCCCCCCCOC(=O)CCCCCN(CCO)CCCCCCCC(=O)OC(CCCCCCCC)CCCCCCCC]

Lipid 5; (nonyl 8-[(8-heptadecan-9-yloxy-8-oxooctyl)-(2-hydroxyethyl) amino]octanoate) WO2017/099823

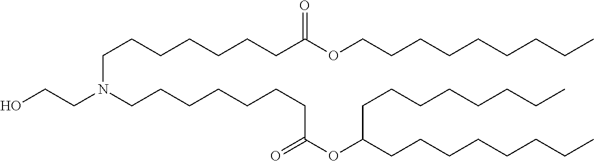 [SMILES: CCCCCCCCCOC(=O)CCCCCCCN(CCO)CCCCCCCC(=O)OC(CCCCCCCC)CCCCCCCC]

Lipid 29; (undecan-3-yl 8-[(8-heptadecan-9-yloxy-8-oxooctyl)-[3-[[2-(methylamino)-3,4-dioxocyclobuten-1-yl]amino]propyl]amino]octanoate)
Adv. Funct. Mater. 2021, 2106727, DOI: 10.1002/adfm.202106727

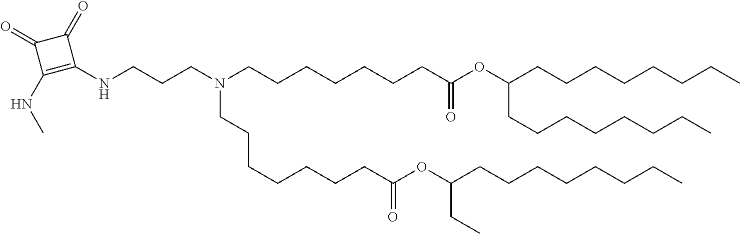 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

ATX-100; (pentadecan-8-yl 4-[3-(dimethylamino)propylsulfanylcarbonyl-(4-oxo-4-pentadecan-8-yloxybutyl)amino]butanoate)
WO2019/191780

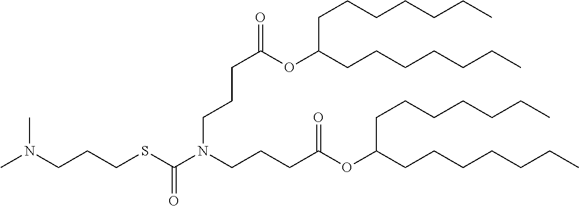 [SMILES: CCCCCCCC(CCCCCCC)OC(=O)CCCN(CCCC(=O)OC(CCCCCCC)CCCCCCC)C(=O)SCCCN(C)C]

Lipid A9; (bis(2-butyloctyl) 10-[3-(dimethylamino)propyl-nonanoylamino] nonadecanedioate) WO2017/004143

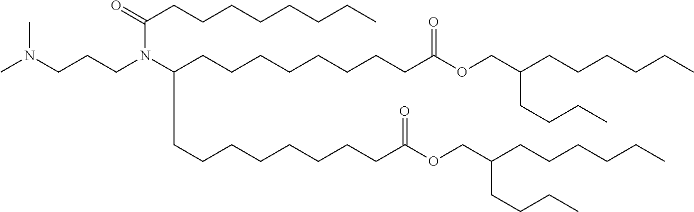 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

Lp01; ([2-[3-(diethylamino)propoxycarbonyloxymethyl]-3-(4,4-dioctoxybutanoyloxy)propyl] (9Z,12Z)-octadeca-9,12-dienoate) WO2015/09534, WO2020/219876

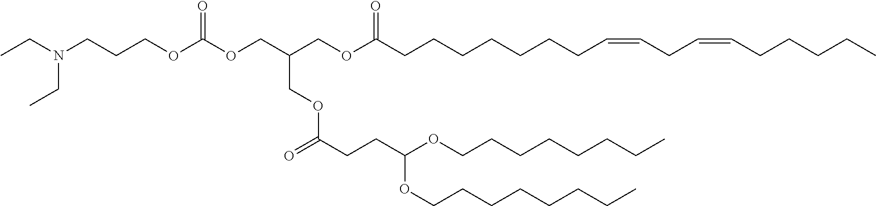 [SMILES: CCCCC/C=C\C/C=C\CCCCCCCC(=O)OCC(COC(=O)CCC(OCCCCCCCC)OCCCCCCCC)COC(=O)OCCCN(CC)CC]

TCL053; ([2-[4-(dimethylamino)butanoyloxymethyl]-3-[(Z)-tetradec-9-enoyl]oxy-2-[[(Z)-tetradec-9-enoyl]oxymethyl]propyl](Z)-tetradec-9-enoate)
WO 2020/032184

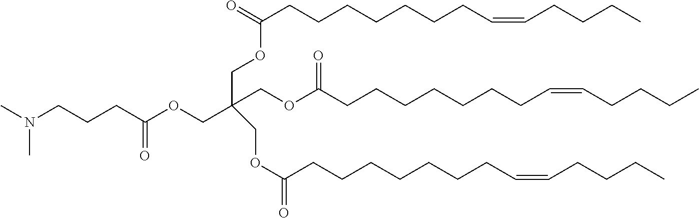 [SMILES: CCCC/C=C\CCCCCCCC(=O)OCC(COC(=O)CCCCCCC/C=C\CCCC)(COC(=O)CCCCCCC/C=C\CCCC)COC(=O)CCCN(C)C]

TCL065; ([2-[5-(dimethylamino)pentanoyloxymethyl]-3-(3-pentyloctanoyloxy)-2-(3-pentyloctanoyloxymethyl)propyl]3-pentyloctanoate)
WO 2020/032184

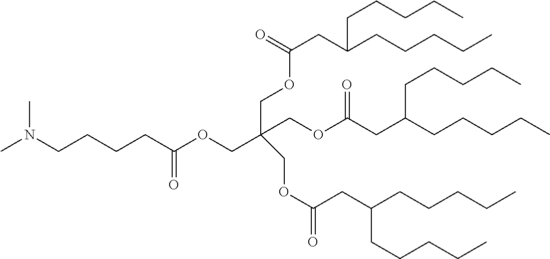 [SMILES: CCCCCC(CCCCC)CC(=O)OCC(COC(=O)CCCCN(C)C)(COC(=O)CC(CCCCC)CCCCC)COC(=O)CC(CCCCC)CCCCC]

Lipid 9; ([(6Z,16Z)-12-[6-(dimethylamino)hexanoyloxy]docosa-6,16-dien-11-yl](Z)-undec-5-enoate)
WO2021/188389

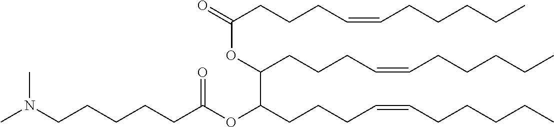 [SMILES: CCCCC/C=C\CCCC(=O)OC(CCC/C=C\CCCCC)C(CCC/C=C\CCCCC)OC(=O)CCCCCN(C)C]

Lipid 19; ([(6Z,16Z)-12-[6-(dimethylamino)hexanoyloxy]docosa-6,16-dien-11-yl] (9Z,12Z)-octadeca-9,12-dienoate)
WO2021/188389.

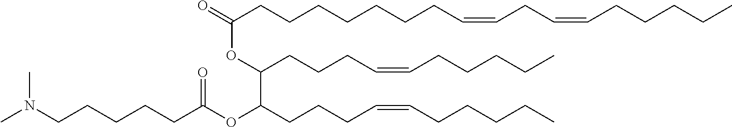 [SMILES: CCCCC/C=C\C/C=C\CCCCCCCC(=O)OC(CCC/C=C\CCCCC)C(CCC/C=C\CCCCC)OC(=O)CCCCCN(C)C]

and
13-B43; ([(6Z,16Z)-12-[(Z)-dec-4-enyl]docosa-6,16-dien-11-yl]5-(dimethylamino)pentanoate)
WO2020/219941

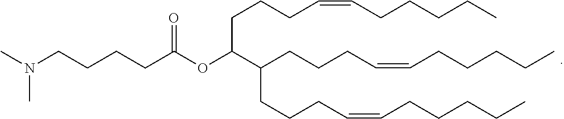 [SMILES: CCCCC/C=C\CCCC(CCC/C=C\CCCCC)C(CCC/C=C\CCCCC)OC(=O)CCCCN(C)C]

Other ionizable lipids suitable for use in the present disclosure include: 306Oi10; (8-methylnonyl 3-[3-[3-[bis[3-(8-methylnonoxy)-3-oxopropyl]amino]propyl-methylamino]propyl-[3-(8-methylnonoxy)-3-oxopropyl]amino]propanoate)

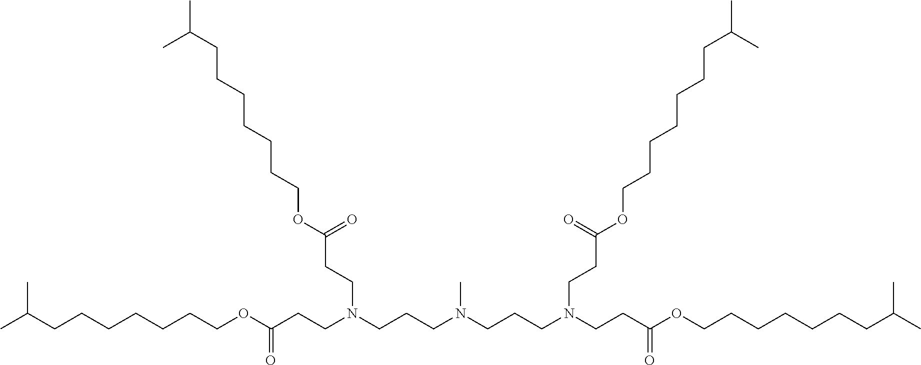 [SMILES: CC(C)CCCCCCCOC(=O)CCN(CCCN(C)CCCN(CCC(=O)OCCCCCCCC(C)C)CCC(=O)OCCCCCCCC(C)C)CCC(=O)OCCCCCCCC(C)C]

93-O17S; (2-tetradecylsulfanylethyl 3-[3-imidazol-1-ylpropyl-[3-oxo-3-(2-tetradecylsulfanylethoxy)propyl]amino]propanoate)

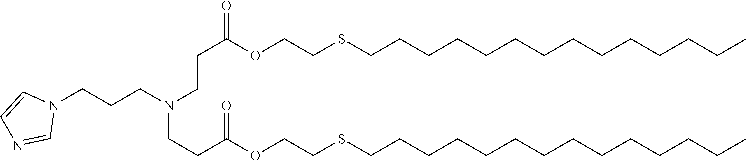 [SMILES: CCCCCCCCCCCCCCSCCOC(=O)CCN(CCCn1ccnc1)CCC(=O)OCCSCCCCCCCCCCCCCC]

93-0170; (2-tetradecoxyethyl 3-[3-imidazol-1-ylpropyl-[3-oxo-3-(2-tetradecoxyethoxy)propyl]amino]propanoate)

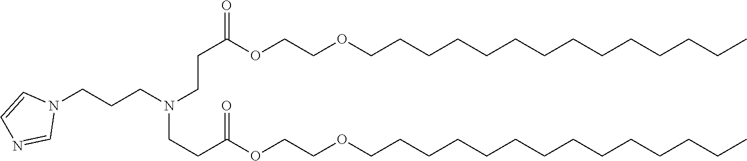 [SMILES: CCCCCCCCCCCCCCOCCOC(=O)CCN(CCCn1ccnc1)CCC(=O)OCCOCCCCCCCCCCCCCC]

80-016B; (2-(dodecyldisulfanyl)ethyl 3-[3-(dimethylamino)propyl-[3-[2-(dodecyldisulfanyl)ethoxy]-3-oxopropyl]amino]propanoate)

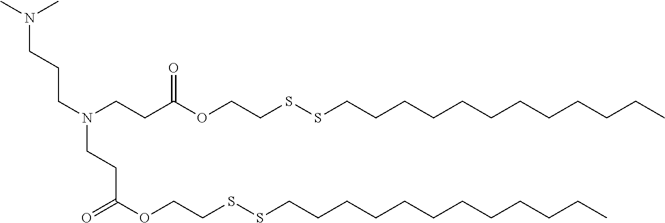 [SMILES: CCCCCCCCCCCCSSCCOC(=O)CCN(CCCN(C)C)CCC(=O)OCCSSCCCCCCCCCCCC]

CL4H6; ([7-[4-(dipropylamino)butyl]-7-hydroxy-13-[(Z)-octadec-9-enoylJoxytridecyl] (Z)-octadec-9-enoate)

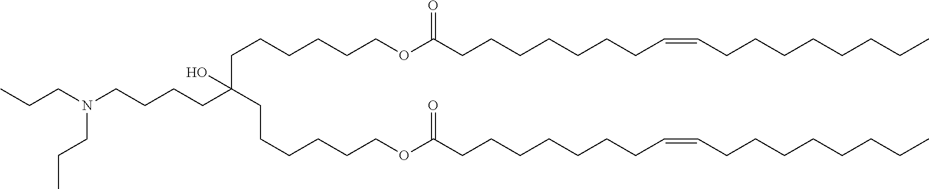 [SMILES: CCCCCCCC/C=C\CCCCCCCC(=O)OCCCCCCC(O)(CCCCCCOC(=O)CCCCCCC/C=C\CCCCCCCC)CCCCN(CCC)CCC]

YSK05; (1-methyl-4,4-bis[(9Z,12Z)-octadeca-9,12-dienoxy]piperidine)

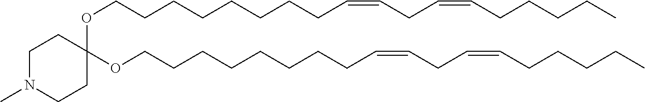 [SMILES: CCCCC/C=C\C/C=C\CCCCCCCCOC1(OCCCCCCCC/C=C\C/C=C\CCCCC)CCN(C)CC1]

Lipid C24; (2-octyldodecyl 3-[4-(4-methylpiperazin-1-yl) butyl-[3-(2-octyldodecoxy)-3-oxopropyl]amino]propanoate)

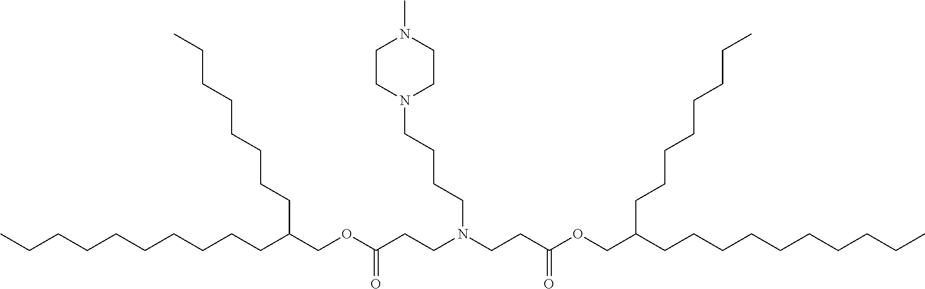 [SMILES: CCCCCCCCCCC(CCCCCCCC)COC(=O)CCN(CCCCN1CCN(C)CC1)CCC(=O)OCC(CCCCCCCC)CCCCCCCCCC]

and
5A2-SC8; bis(2-((2-methyl-3-(octylthio)propanoyl)oxy) ethyl) 4,10,16-tris(3-(2-((2-methyl-3-(octylthio)propanoyl)oxy)ethoxy)-3-oxopropyl)-4,7,10,13,16-pentaazanonadecanedioate (5 ionizable amine groups+10 ester groups)

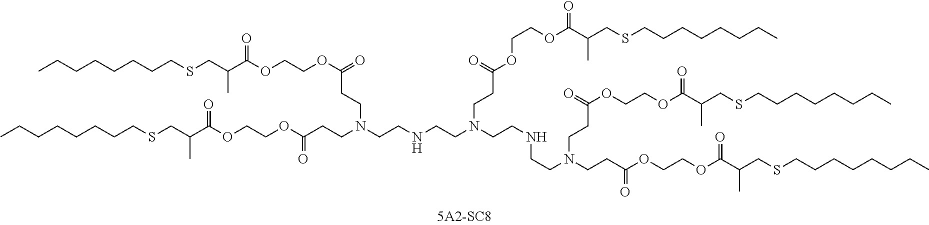 [SMILES: CCCCCCCCSCC(C)C(=O)OCCOC(=O)CCN(CCNCCN(CCC(=O)OCCOC(=O)C(C)CSCCCCCCCC)CCC(=O)OCCOC(=O)C(C)CSCCCCCCCC)CCNCCN(CCC(=O)OCCOC(=O)C(C)CSCCCCCCCC)CCC(=O)OCCOC(=O)C(C)CSCCCCCCCC]

Additional ionizable lipids suitable in the present disclosure include:
cKK-E12, MD1; (3,6-bis [4-[bis(2-hydroxydodecyl)amino]butyl]piperazine-2,5-dione)

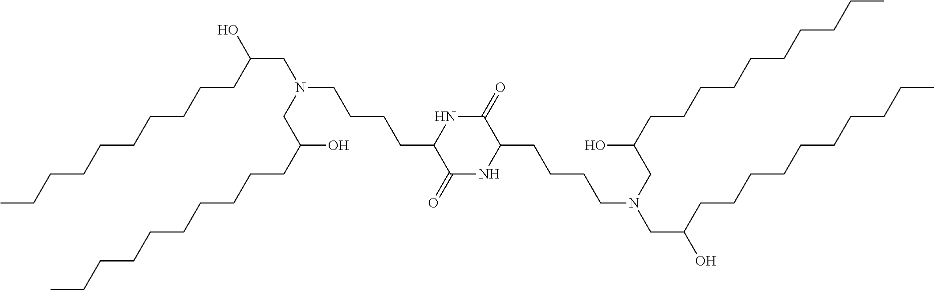 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

C12-200; (1-[2-[bis(2-hydroxydodecyl)amino]ethyl-[2-[4-[2-[bis (2-hydroxydodecyl)amino]ethyl]piperazin-1-yl]ethyl]amino]dodecan-2-ol)

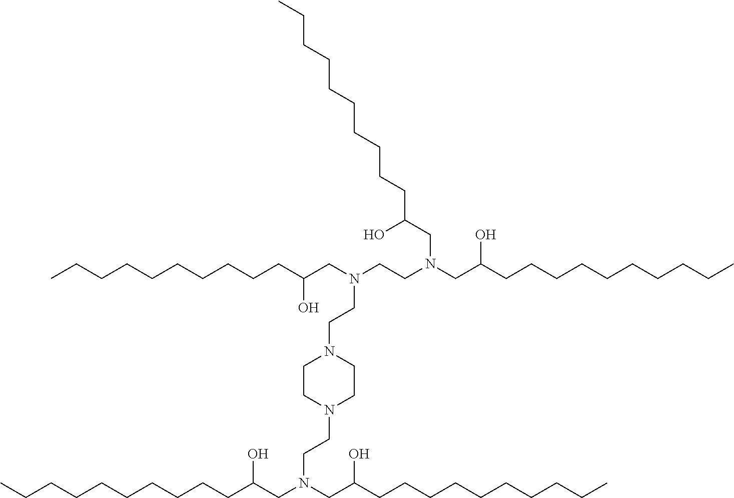 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

### E. Cationic Lipids

Cationic lipids suitable for use in a composition of the present disclosure include, for example, a variety of cationic lipids. As used herein “cationic lipids”, comprise at least one permanently positively charged moiety. The permanently positively charged moiety may be positively charged at a physiological pH such that the cationic lipid comprises a positive charge upon delivery of an agent or therapeutic agent to a cell. In some embodiments the positively charged moiety is quaternary amine or quaternary ammonium ion. In some embodiments, the cationic lipid comprises, or is otherwise complexed to or interacting with, a counterion. Cationic lipids useful in the present disclosure comprise one or more hydrophobic components and a permanently cationic group. The permanently cationic lipid may contain a group which has a positive charge regardless of the pH. One permanently cationic group that may be used in the permanently cationic lipid is a quaternary ammonium group.

The cationic lipid component of the lipid nanoparticle of the present invention may comprise of between 1 and 99 molar percent of the lipid molecules in the nanoparticle. In other embodiments, the cationic lipid component may comprise of between 5 and 90 molar percent of the lipid molecules in the lipid nanoparticles. In some embodiments the cationic lipid component comprises between 10 and 75, 10 and 60, 10 and 50, or 15 and 40 molar percent of the lipid molecules in the lipid nanoparticles.

In some embodiments, a cationic lipid suitable for use in the present disclosure is biodegradable. A cationic lipid suitable for use in the present disclosure may have one or more biodegradable groups located in a hydrophobic section of the cationic lipid (e.g. within a hydrocarbon chain). The incorporation of biodegradable group(s) into the cationic lipid may result in faster metabolism and removal of the cationic lipid from the body following delivery of the active agent to a target area. As a result, these cationic lipids may have lower toxicity than similar cationic lipids without the biodegradable groups. These cationic lipids may be incorporated into a lipid particle for delivering an active agent, such as a nucleic acid.

A cationic lipid for use in the present disclosure may be a lipid of formula (IV):

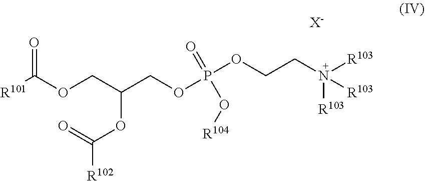

wherein:
R¹⁰¹ and R¹⁰² are each independently alkyl (C₈-C₂₄), alkenyl (C₈-C₂₄), or a substituted version of either group;
R¹⁰³ is independently in each instance alkyl (C₁-C₆) or substituted alkyl (C₁-C₆);
R¹⁰⁴ is alkyl (C₁-C₆) or substituted alkyl (C₁-C₆); and
X⁻ is a monovalent anion.

Examples of cationic lipids suitable for use in the present disclosure include, but are not limited to: 14:1 EPC (1,2-dimyristoleoyl-sn-glycero-3-ethylphosphocholine), particularly as the OTf salt; EDLPC (1,2-dilauroyl-sn-glycero-3-ethylphosphocholine), particularly as the chloride salt; EDMPC (1,2-dimyristoyl-sn-glycero-3-ethylphosphocholine), particularly as the chloride salt; EDPPC (1,2-dipalmitoyl-sn-glycero-3-ethylphosphocholine), particularly as the chloride salt; EDSPC (1,2-distearyol-sn-glycero-3-ethylphosphocholine), particularly as the chloride salt; EDOPC (1,2-dioleoyl-sn-glycero-3-ethylphosphocholine), particularly as the chloride salt; and 16:0-18:1 EPC (1-palmitoyl-2-oleoyl-sn-glycero-3-ethylphosphocholine), particularly as the chloride salt.

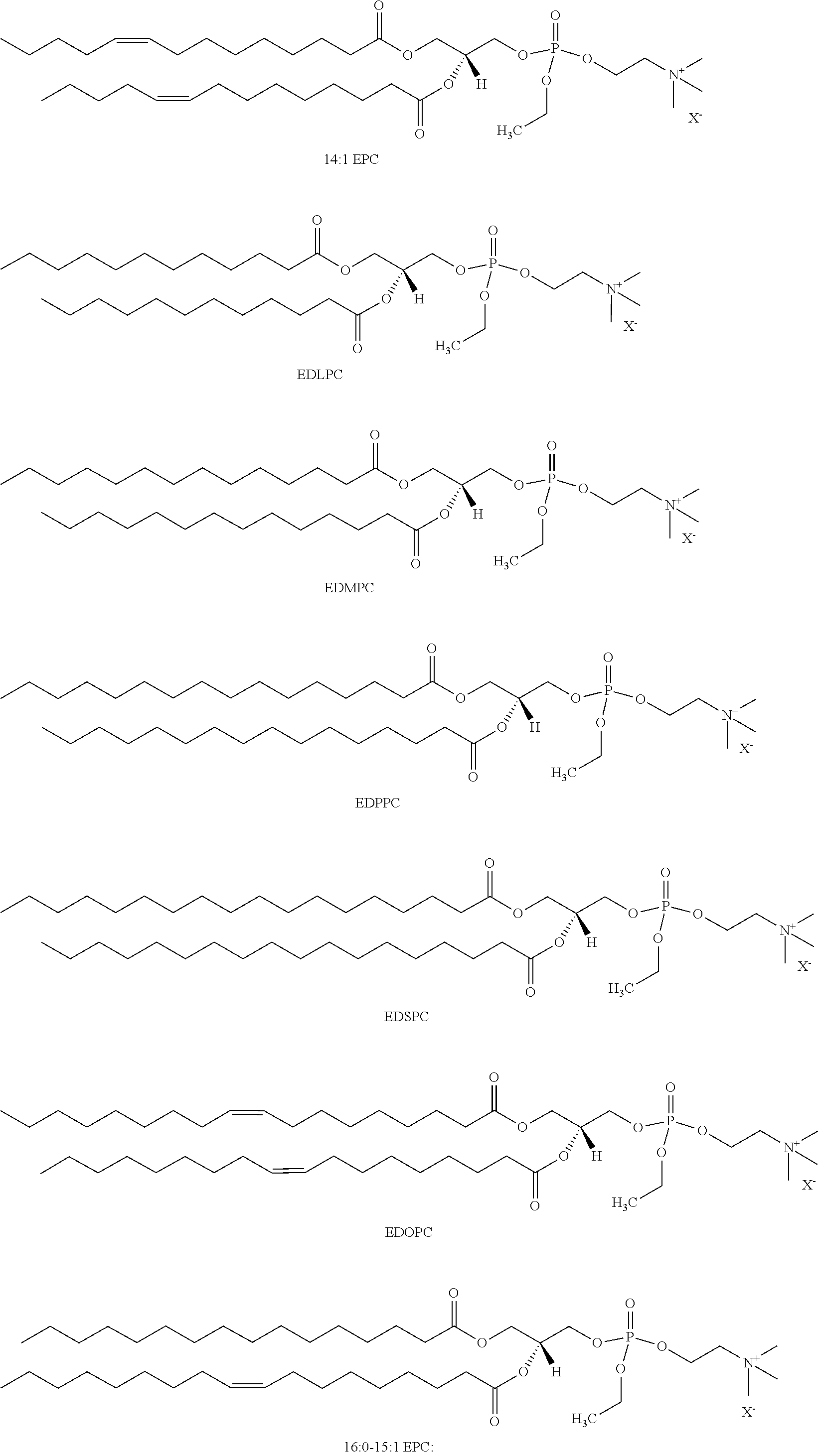

In some embodiments, a cationic lipid suitable for use in the present disclosure is a cationic lipid of formula (V):

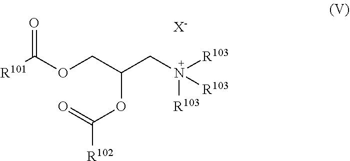

wherein:
R¹⁰¹ and R¹⁰² are each independently alkyl (C₈-C₂₄), alkenyl(C₈-C₂₄), or a substituted version of either group;
R¹⁰³ is independently in each instance alkyl (C₁-C₆) or substituted alkyl (C₁-C₆); and
X⁻ is a monovalent anion.

Additional examples of cationic lipids suitable for use in the present disclosure include, but are not limited to: DMTAP (1,2-dimyristoyl-3-trimethylammonium-propane), particularly as the chloride salt; DPTAP (1,2-dipalmitoyl-3-trimethylammonium-propane), particularly as the chloride salt; DSTAP (1,2-distearyol-3-trimethylammonium-propane), particularly as the chloride salt; DOTAP (1,2-dioleoyl-3-trimethylammonium-propane), particularly as the chloride salt; and DORI (N-(2-hydroxyethyl)-N,N-dimethyl-2,3-bis(oleoyloxy)propan-1-aminium), particularly as the bromide salt.

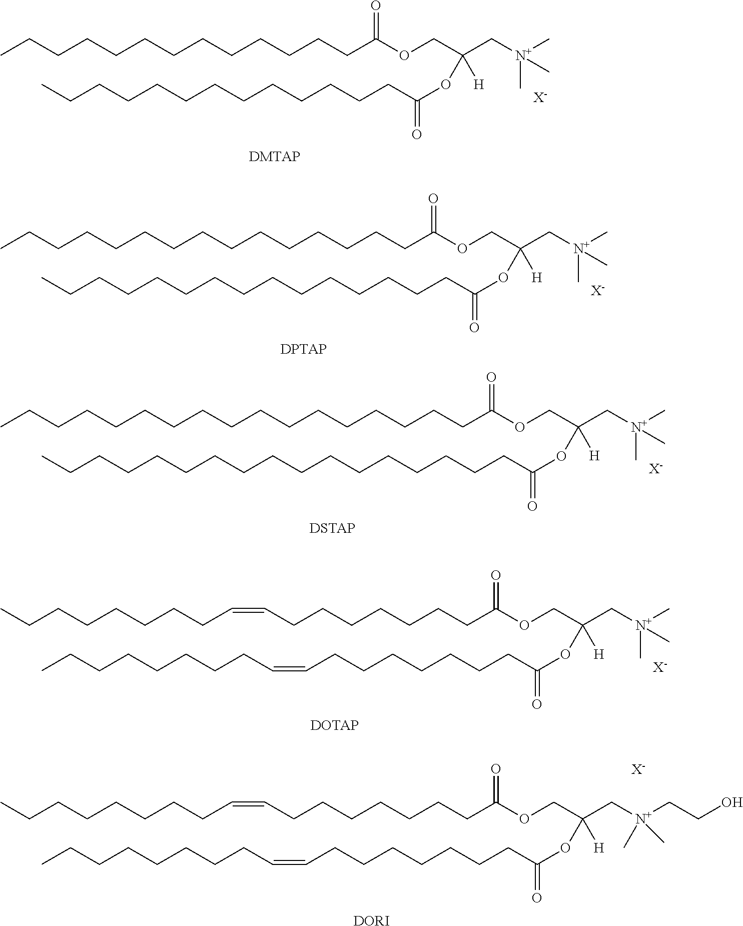

In some embodiments, a cationic lipid suitable for use in the present disclosure is a lipid of formula (VI):

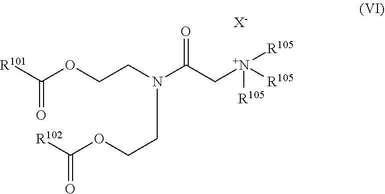

wherein:
R¹⁰¹ and R¹⁰² are each independently alkyl(C₈-C₂₄), alkenyl(C₈-C₂₄), or a substituted version of either group;
R¹⁰⁵ are each independently alkyl (C₁-C₆); and
X⁻ is a monovalent anion.

An additional example of a cationic lipid suitable for use in the present disclosure is DC-6-14 (O,O′-ditetradecanoyl-N-(a-trimethylammonioacetyl) diethanolamine), particularly as the chloride salt.

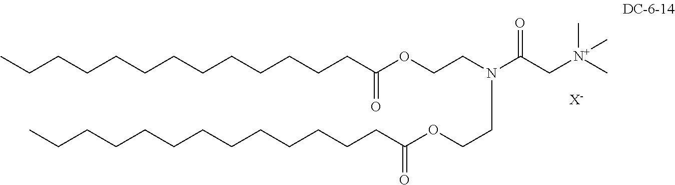

### F. Phospholipids

In some embodiments, the compositions of the present disclosure comprise a phospholipid. As used herein “phospholipid” refers to a lipid molecule comprising at least one hydrocarbon tail group, a glycerol or sphingosine moiety, and optionally a small organic moiety preferentially selected from an amino acid, a sugar, choline, or ethanolamine. Phospholipids of the present disclosure, such as those containing choline, ethanolamine, or an amino acid moiety, may have a positively charged group. However, phospholipids as defined herein are distinct from ionizable lipids or cationic lipids as defined herein due to the presence of a negative charge on the lipid molecule. Phospholipids of the present disclosure are therefore zwitterionic, and the term “phospholipid” can be understood to be synonymous with “zwitterionic phospholipid” as used herein.

The phospholipid component of the lipid nanoparticle of the present invention may comprise of between 0.1 and 99 molar percent of the lipid molecules in the nanoparticle. In other embodiments, the phospholipid component may comprise of between 0.1 and 75 molar percent of the lipid molecules in the lipid nanoparticles. In some embodiments the phospholipid component comprises between 1 and 50, 1 and 30, 1 and 15, or 5 and 10 molar percent of the lipid molecules in the lipid nanoparticles.

Phospholipids that may be suitable in the present disclosure include, but are not limited to DSPC (1,2-distearoyl-sn-glycero-3-phosphocholine). DMPC (1,2-dimyristoyl-sn-glycero-3-phosphocholine), DPPC (1,2-dipalmitoyl-sn-glycero-3-phosphocholine), DSPC (1,2-distearoyl-sn-glycero-3-phosphocholine), POPC (1-palmitoyl-2-oleoyl-sn-glycero-3-phosphocholine), DOPC (1,2-dioleoyl-sn-glycero-3-phosphocholine), DMPE (1,2-dimyristoyl-sn-glycero-3-phosphoethanolamine), DPPE (1,2-dipalmitoyl-sn-glycero-3-phosphoethanolamine), DSPE (1,2-distearoyl-sn-glycero-3-phosphoethanolamine), DOPE (1,2-dioleoyl-sn-glycero-3-phosphoethanolamine), DLoPE (1,2-dilinoleoyl-sn-glycero-3-phosphoethanolamine), D (Phy) PE (1,2-diphytanoy 1-sn-glycero-3-phosphoethanolamine), POPE (1-palmitoyl-2-oleoyl-sn-glycero-3-phosphoethanolamine), 1,2-ditetradecyl-sn-glycero-3-phosphoethanolamine, 1,2-dihexadecyl-sn-glycero-3-phosphoethanolamine, 1,2-dioctadecyl-sn-glycero-3-phosphoethanolamine, 1,2-diphytanyl-sn-glycero-3-phosphoethanolamine, and the like. In some embodiments the phospholipid is a phosphatidylcholine. In some embodiments the phospholipid is a phosphatidylethanolamine. In some embodiments, the phospholipid is DSPC.

### G. Sterols

In some embodiments, the compositions of the present disclosure comprise a sterol. As used herein, “sterols” refer to steroids and steroid derivatives, where the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as in the structure of cholesterol.

In some embodiments, a sterol suitable in the present disclosure is a derivative of cholestane. As described above, a cholestane derivative includes one or more non-alkyl substitution on the fused ring system. In some embodiments, the cholestane or cholestane derivative is a cholestene or cholestene derivative.

Sterols that may be suitable in the present disclosure include, but are not limited to cholesterol, sitosterol, stigmasterol, fucosterol, spinasterol, brassicasterol, ergosterol, cholestanone, cholestenone, coprostanol, cholesteryl-2′-hydroxyethyl ether, cholesteryl-4′-hydroxybutyl ether and the like. In some embodiments, the sterol of the present disclosure is a phytosterol. In some embodiments, the sterol of the present disclosure is cholesterol.

The Sterol component of the lipid nanoparticle of the present invention may comprise of between 1 and 99 molar percent of the lipid molecules in the nanoparticle. In other embodiments, the Sterol component may comprise of between 1 and 75 molar percent of the lipid molecules in the lipid nanoparticles. In some embodiments the Sterol component comprises between 5 and 75, 5 and 60, 10 and 50, or 15 and 45 molar percent of the lipid molecules in the lipid nanoparticles.

### H. Peg-Lipids

In some embodiments, the compositions of the present disclosure comprise a PEG-lipid. As used herein, “PEG-lipid” refers to a polyethylene glycol (PEG) polymer attached to one or more hydrocarbon chains 1-30 carbon atoms in length. In some embodiments, a PEG lipid is a compound one or more hydrocarbon chains 1-30 carbon atoms in length attached to a linker group which is also attached to the PEG chain. In some embodiments, the PEG lipid is a diglyceride which also comprises a PEG chain attached to the glycerol group.

The PEG lipid component of the lipid nanoparticle of the present invention may comprise of between 0.1 and 99 molar percent of the lipid molecules in the nanoparticle. In other embodiments, the PEG lipid component may comprise of between 0.1 and 75 molar percent of the lipid molecules in the lipid nanoparticles. In some embodiments the PEG lipid component comprises between 0.1 and 25, 0.1 and 10, 0.1 and 5, or 0.1 and 3 molar percent of the lipid molecules in the lipid nanoparticles.

Some non-limiting examples of a PEG lipid include a PEG modified phosphatidylethanolamine or phosphatidic acid, a PEG-ceramide conjugate, PEG modified dialkylamines, PEG modified 1,2-diacyloxypropan-3-amines, PEG modified diacylglycerols and dialkylglycerols. In some embodiments, a PEG lipid suitable for use in the disclosure is a PEG modified distearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. In some embodiments, the PEG modification is measured by the number or repeated subunits in the polymer chain. Some non-limiting examples of lipids that may be used in the present application are taught by U.S. Pat. No. 5,820,873, WO 2010/141069, or U.S. Pat. No. 8,450,298, which is incorporated herein by reference.

PEG-lipids that may be suitable for the present disclosure include, but are not limited to DMG-mPEG2000 (1,2-dimyristoyl-rac-glycero-3-methoxypolyethylene glycol-2000), DPG-mPEG2000 (1,2-dipalmitoyl-rac-glycero-3-methoxypolyethylene glycol-2000), DSG-mPEG2000 (1,2-distearoyl-rac-glycero-3-methoxypolyethylene glycol-2000). While PEG modification may incorporate the PEG group non-regiospecifically, and the number of repeated monomers varies such that the listed molecular weight represents an average value, a typical structure represented by DMG-mPEG2000 includes for example:

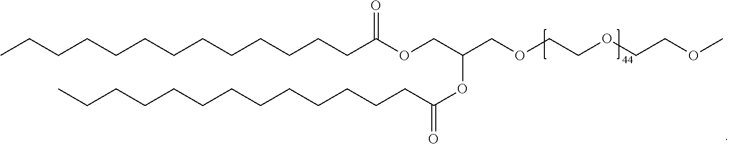

### I. Administration

Compositions of the present disclosure comprise a plurality of lipid particles (e.g., lipid nanoparticles, such as any of the lipid nanoparticles described herein). In some embodiments, the compositions are pharmaceutical compositions. In some embodiments, the compositions further comprise a pharmaceutically acceptable carrier.

A “pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block, polymers, polyethylene glycol and wool fat.

Compositions described herein may be administered parenterally (including subcutaneously, intramuscularly, intravenously and intradermally), by inhalation spray, orally, topically, rectally, nasally, buccally, vaginally or via an implanted reservoir. The terms “parenteral” and “parenterally,” as used herein, include subcutaneous, intracutaneous, intravenous, intramuscular, intraocular, intravitreal, intraarticular, intra-arterial, intra-synovial, intrasternal, intrathecal, intralesional, intrahepatic, intraperitoneal, intralesional and intracranial injection or infusion techniques. In some aspects, a composition described herein is administrable intravenously and/or intraperitoneally. In some aspects, a composition described herein is administrable orally. In some aspects, a composition described herein is administrable subcutaneously. Preferably, a composition described herein is administered orally, subcutaneously, intraperitoneally or intravenously. More preferably, a composition described herein is administered intravenously.

Compositions described herein can also be administered subcutaneously, intraperitoneally or intravenously, e.g., in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, dextrose, water, phosphate buffered saline, Ringer's solution, lactated Ringer's solution and isotonic sodium chloride solution. Commonly used surfactants such as Tweens or Spans and/or other similar emulsifying agents or bioavailability enhancers which are commonly used in the manufacture of pharmaceutically acceptable solid, liquid, or other dosage forms can also be used for the purposes of formulation.

Administration can be local or systemic as indicated. In some embodiments, administration (e.g., of a composition of the disclosure) is oral. In some embodiments, administration (e.g., of a composition of the disclosure) is intravenous. The preferred mode of administration can vary depending on the particular composition or agent to be delivered.

### J. Methods of Use

The lipid compositions of the present disclosure may be used to deliver agents to various tissues and organs within a subject. In some aspects, the present disclosure describes a method of delivering an agent to one or more of the brain, heart, muscles, and kidneys of a subject in need thereof, comprising systemically administering to the subject a composition comprising a plurality of lipid nanoparticles, wherein the lipid nanoparticles comprise the agent, an ionizable lipid and a fully saturated cationic lipid. In some embodiments, the agent is delivered to the brain. In some embodiments, the agent is delivered to the muscles. In some embodiments, the agent is delivered to the heart. In some embodiments, the agent is delivered to the kidneys. In some embodiments, the agent is delivered to the heart and muscles. In some embodiments, the agent is delivered to the heart and brain. In some embodiments, the agent is delivered to the heart and kidneys. In some embodiments, the agent is delivered to the brain and muscles. In some embodiments, the agent is delivered to the brain and kidneys. In some embodiments, the agent is delivered to the muscles and kidneys. In some embodiments, the agent is delivered to the muscles, kidneys, and heart. In some embodiments, the agent is delivered to the muscles, kidneys, and brain. In some embodiments, the agent is delivered to the brain, kidneys, and heart. In some embodiments, the agent is delivered to the muscles, brain, and heart.

In some aspects, the present disclosure describes a method of delivering an agent to a subject in need thereof, comprising administering the subject a composition of the present disclosure. In some aspects, the present disclosure describes a method of treating a subject having a disease, disorder or condition beneficially treated by an agent, comprising administering to the subject a therapeutically effective amount of a composition of the present disclosure. In some embodiments, the ionizable lipid in the administered composition is an ionizable lipid of formula (I). In some embodiments, the ionizable lipid in the administered composition is an ionizable lipid of formula (II). In some embodiments, the ionizable lipid in the administered composition is an ionizable lipid of formula (III). In some embodiments, the ionizable lipid in the administered composition is an ionizable lipid of formula (VII). In some embodiments, the ionizable lipid in the administered composition is an ionizable lipid of formula (VIII). In some embodiments, the ionizable lipid in the administered composition is an ionizable lipid of formula (IX). In some embodiments, the ionizable lipid in the administered composition is an ionizable lipid of formula (X). In some embodiments, the cationic lipid in the administered composition is a cationic lipid of formula (IV). In some embodiments, the cationic lipid in the administered composition is a cationic lipid of formula (V). In some embodiments, the cationic lipid in the administered composition is a cationic lipid of formula (VI).

Typically, a composition of the disclosure will be administered every 1 to about 30 days. In some embodiments, the administration is every 30 days. In some embodiments, the administration is every 25 days. In some embodiments, the administration is every 20 days. In some embodiments, the administration is every 10 days. In some embodiments, the administration is every 9 days. In some embodiments, the administration is every 8 days. In some embodiments, the administration is every 7 days. In some embodiments, the administration is every 6 days. In some embodiments, the administration is every 5 days. In some embodiments, the administration is every 4 days. In some embodiments, the administration is every 3 days. In some embodiments, the administration is every 2 days. In some aspects, the administration (e.g., of a composition of the disclosure) is QD or BID (e.g., QD)). In some aspects, the administration (e.g., of a composition of the disclosure) is daily.

In some aspects, a composition described herein further includes one or more additional agents, e.g., for use in combination with the first agent. Some embodiments provide a combination (e.g., pharmaceutical combination) comprising an agent in addition to one or more therapeutic agents. Such combinations are particularly useful as, for example, when the first agent and the one or more additional therapeutic agents are to be administered separately. In a combination provided herein, the two or more agents can be administrable by the same route of administration or by different routes of administration. In some embodiments, the additional agent is a therapeutic agent. In some embodiments, the additional agent is a diagnostic agent. In some embodiments, the agent is an imaging agent. In some aspects, the additional agent is a small molecule. In some aspects, the additional agent is a peptide. In some aspects, the additional agent is a protein. In some aspects, the additional agent is an antibody or antibody fragment.

In some embodiments of the disclosure, the agent in the composition is a therapeutic agent for the treatment of a disease, disorder or condition. A composition of the disclosure can also be administered in combination with one or more additional therapies to treat a disease, disorder or condition. When administered “in combination” with such additional therapies, the compound of the disclosure can be administered before, after or concurrently with the other therapy(ies) (e.g., additional therapeutic agent(s)). When administered simultaneously (e.g., concurrently), the composition of the disclosure and another therapy can be in separate formulations or the same formulation. Further, the additional therapy may be in the solvent of the formulation or associated with the lipid nanoparticle, including attached to the surface by covalent or non-covalent bonding, embedded in the membrane or encased in the nanoparticle. Alternatively, the composition of the disclosure and another therapy can be administered sequentially, either at approximately the same time or at different times, as separate formulations. When the composition of the disclosure and the other therapy (e.g., therapeutic agent) are administered as separate formulations or compositions, the composition of the disclosure and the other therapy can be administered by the same route of administration or by different routes of administration. A skilled clinician can determine appropriate timing for administration of each therapy being used in combination (e.g., timing sufficient to allow an overlap of the pharmaceutical effects of the therapies). Typically, a combination therapy will provide beneficial effects of the therapeutic agent combination in treating the diseases, conditions or disorders described herein.

A composition of the disclosure or other therapeutic agent can be administered in a dosage ranging from about 0.001 mg/kg to about 100 mg/kg of body weight or, alternatively, in a dosage ranging from about 1 mg/dose to about 5,000 mg/dose, or according to the requirements of the particular agent. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. In some aspects, a suitable dosage is from about 0.1 mg/kg to about 10 mg/kg, e.g., from about 0.1 mg/kg to about 5 mg/kg, from about 0.1 mg/kg to about 2.5 mg/kg or about 0.1 mg/kg to about 1.0 mg/kg, body weight per treatment. Suitable dosages can be from about 0.001 mg/dose to about 100 mg/dose, from about 0.01 mg/dose to about 100 mg/dose, from about 0.01 mg/dose to about 50 mg/dose, from about 0.1 mg/dose to about 10 mg/dose, from about 0.05 mg/dose to about 50 mg/dose, from about 0.1 mg/dose to about 100 mg/dose, from about 1 mg/dose to about 7,500 mg/dose, from about 1 mg/dose to about 5,000 mg/dose, from about 10 mg/dose to about 2,500 mg/dose or from about 100 mg/dose to about 1,000 mg/dose.

Doses lower or higher than those recited above may be required. Specific dosage and treatment regimens for any particular patient will depend, for example, upon a variety of factors, such as the activity of the specific agent employed, the age, body weight, general health status, sex, diet, time of administration, frequency of administration, rate of excretion, drug combination, the severity and course of the disease, condition or symptoms, the subject's disposition to the disease, condition or symptoms, and the judgment of the treating physician. Determining the dosage for a particular agent, subject and disease, disorder or condition is within the abilities of one of skill in the art.

### K. Exemplification

### Materials and Methods:

All chemicals obtained from commercial sources were stored following the manufacturer's instruction and used without further purification. Lipids represented by formula (I) or (III) or (VII) and a method for producing the same are described in US2021/0085604 and US2022/0273817. The entirety of each publication is hereby incorporated by reference.

siRNA: One of the most common evaluation systems for extrahepatic delivery is bioluminescence imaging (BLI) using Firefly luciferase (FLuc) mRNA. Upon administering a nontoxic, stable substrate, FLuc emits luminescence at tissue-penetrating wavelengths that can be imaged in vivo and ex vivo, making it useful to identify tissues where mRNA is successfully delivered and translated. Although these BLI techniques are simple and convenient, quantification of bioluminescence from bulk tissue may overlook RNA delivery to cell types that are underrepresented in the tissue. This is especially true for endothelial cells. For example, lungs and liver are composed of 15-30% endothelial cells, whereas kidney and brain are composed of less than 5% endothelial cells, resulting in small bioluminescence even with efficient FLuc mRNA delivery to endothelial cells. In principle, mRNAs encoding fluorescent proteins can facilitate the cell-type level analysis by microscopy and flow cytometry. However, it has been reported that currently commercially available GFP and tdTomato mRNAs cannot induce sufficient protein expression to be visualized in vivo beyond background fluorescence. To address this sensitivity issue, an evaluation system was developed in which Cre recombinase mRNA is administered to genetically engineered mice (Ai14) in which transcription of the CAG promoter-driven tdTomato protein is inhibited by a loxP-flanked STOP cassette, allowing highly sensitive evaluation. However, the Ai14/Cre mRNA model is an “on/off” binary system in which successful transfection of one Cre mRNA results in the same tdTomato expression as many Cre mRNAs, and the efficiency of RNA delivery (input) and fluorescence intensity and protein expression (output) are not proportional.

Instead, siRNA is a useful therapeutic modality as well as a powerful tool for LNP in vivo screening. For example, early efforts to improve the potency of ionizable lipids were supported by a screening system using siRNA against Factor VII (FVII), a blood clotting factor secreted by hepatocytes. Since FVII expression is strictly limited to hepatocytes, RNA delivery efficiency to hepatocytes can be easily evaluated by quantifying serum FVII protein level. Regarding endothelial cells, using siRNAs against genes specifically expressed in endothelial such as Icam2, Tie2, and Cdh5 can be effective.

Cadherin (Cdh5), also known as vascular endothelial cadherin (VE-cadherin) is a useful target gene to evaluate endothelial cell siRNA delivery in that Cdh5 expression is strictly limited to endothelial cells. By quantifying Cdh5 mRNA remaining in the tissues after siCdh5 administration, RNA delivery efficiency to endothelial cells can be evaluated.

The following custom siRNA was manufactured by Horizon. siRNA against murine VECadherin (siVEcad, siCdh5):

| | Sense: |
|---|---|
| (SEQ ID NO: 1) | |
| | 5′-mCmCAAAAGAGAGAmCmUGGAmUmUdTsdT-3′ |
| | Antisense: |
| (SEQ ID NO: 2) | |
| | 5′-AAUCmCAGUCUCUCUUUUGGdTsdT-3′ |

### Abbreviation

A Adenosine-3′-phosphate
C Cytidine-3′-phosphate
G Guanonsine-3′-phosphate
U Uridine-3′-phosphate
mA 2′-O-methyladenosine-3′-phosphate
mC 2′-O-methylcytidine-3′-phosphate
mG 2′-O-methylguanonsine-3′-phosphate
mU 2′-O-methyluridine-3′-phosphate
dT 2′-deoxythymidine-3′-phosphate
sdT 2′-deoxythymidine-5′-phosphate-phosphorothioate

Lipid Nanoparticle Formulation: Lipid nanoparticles were synthesized using a microfluidics chip device as previously described (Chen, D., J. Am. Chem. Soc. (2012). Rapid discovery of potent siRNA-containing lipid nanoparticles enabled by controlled microfluidic formulation.). Briefly, a lipid-containing ethanol phase was mixed with a siRNA-containing aqueous phase through microfluidic channel in the PDMS (poly-dimethyl-siloxane) chip. siRNA was diluted in 10 mM citrate buffer, pH 3.0, (aqueous phase) while the appropriate amounts of lipids were co-dissolved in 200 proof ethanol (ethanol phase). Syringe pumps were used to mix the ethanol and aqueous phases together at a 1:3 volume ratio (total flow rate 1.2 mL/min).

The resulting LNPs were dialyzed against phosphate-buffered saline (PBS) in a 20 kDa molecular weight cut-off (MWCO) cassette at 4° C. or room temperature overnight and stored at 4° C. until use. In some experiments, LNP solutions were further concentrated using a 100 kDa MWCO Amicon ultra centrifugal filters (Millipore Sigma). For animal experiments, LNP solutions were sterilized through a 0.22 μm filter (Millipore Sigma).

Particle Size and Zeta Potential Measurement: The diameter (z-average) and the polydispersity index (PDI) of the LNPs were measured using dynamic light scattering (Zetasizer Nano ZS (Malvern Instruments)). The zeta potential was measured using the same instrument in a 0.1X PBS solution.

Quantification of siRNA Concentration and Encapsulation: To quantify the siRNA concentration and to determine the RNA encapsulation efficiency, modified Quant-iT RiboGreen RNA assay (Thermo Fisher) was used as previously described (Walsh C. et al. Methods Mol Biol. 2014; 1141:109-20). This assay measures the quantity of RNA in samples with intact LNPs to determine the quantity of unencapsulated RNA as well as in LNP samples disrupted by triton X-100 to quantify the total RNA. Briefly, 0.5 μL of LNPs or serial dilutions of siRNA at known concentrations were diluted in a final volume of 100 μl of TE buffer (10 mM tris-HCl and 20 mM EDTA) in the presence or absence of 2% Triton X-100 (Sigma-Aldrich) in a black 96-well plate. The plate was incubated at 37° C. for 15 minutes with shaking at 350 rpm. Following the incubation, 99.5 μl of TE buffer and 0.5 μl of RiboGreen reagent were added to each well. The plate was incubated at 37° C. for 2 minutes with shaking at 350 rpm. RiboGreen fluorescence was measured (excitation wavelength of 485 nm and emission wavelength of 528 nm) using a plate reader (Tecan). RNA encapsulation efficiency (EE %) was determined via the following equation:

pKa Value Measurement by TNS Assay: The apparent pKa values of lipid nanoparticles were determined using 2-(p-toluidinyl) naphthalene-6-sulphonic acid (TNS) assay as described previously (Heyes J. et al. Journal of Controlled Release 107 (2005) 276-287). Briefly, pH buffers ranging from 3.0 to 9.0 in 0.5 increments were prepared by mixing a solution of 20 mM sodium phosphate buffer, 20 mM sodium citrate buffer, 20 mM sodium borate buffer, and 150 mM NaCl. 300 μM TNS solution was added to the above pH buffer in the final concentration of 6 μM. 46 μL of each pH buffer containing 6 μM TNS was added to a black 384-

$$EE \% = ( 1 - \frac{{RNA}_{TE}}{{RNA}_{TX}} ) \times 100$$

well plate. Lastly, 4 μL of LNP solutions with 500 ng/μL ionizable lipid were added to each well (Quadruplicate). Fluorescence intensity was measured using a plate reader (Tecan) at an excitation of 320 nm and an emission of 465 nm. The resulting pH-fluorescence sigmoidal curves were evaluated by a curve fit analysis using Prism (Graphpad), and the apparent pKa values were obtained as the inflection points of the sigmoidal curves.

Animal Experiments: All animal studies were approved by the MIT Institutional Animal Care and Use Committee (CAC) and were consistent with local, state, and federal regulations as applicable. All experimental procedures were performed with ethical compliance and approval under the guidelines for Division of Comparative Medicine by Massachusetts Institute of Technology. Female C57BL/6 mice (6 weeks) were obtained from Jackson Laboratory, housed in an MIT animal facility, and acclimated for at least 3 days before the initiation of a study.

For intravenous administration, siRNA-lipid nanoparticles diluted in PBS were injected via the tail vein using 29 g, 3/10 cc insulin syringes (BD Biosciences) after gentle warming of the animals using a heat lamp. For intramuscular administration, siRNA-lipid nanoparticles diluted in PBS were injected via the tail vein using 29 g, 3/10 cc insulin syringes (BD Biosciences).

48-72 hours after injection, organs or tissues including the heart, liver, spleen, lung, kidneys, muscle (quadriceps), and brain were collected and soaked in RNAlater solution (Thermo Fisher) for 12-48 hours at 4° C. and stored at −20° C. after the removal of RNAlater.

Tissue mRNA Quantification by RT-qPCR: Total RNA was isolated from tissues using the TRIzol (Thermo Fisher) and Direct-zol-96 MagBead (Zymo Research), or Quick-RNA MagBead (Zymo Research). Briefly, tissue punches were placed in a deep-well 96 well plate with 4-mm stainless beads and lysed with 350 μL TRIzol, or 250 μL DNA/RNA Shield (Zymo Reaearch) using a GenoGrinder2010. Total RNA was further purified using Direct-zol-96 MagBead (Zymo Research) or Quick-RNA MagBead (Zymo Research) according to the manufacturer's protocol.

cDNA was synthesized using ABI High Capacity cDNA Reverse Transcription Kit (Applied Biosystems #4368814) according to manufacturer instructions. Briefly, 5 μl of a master mix containing 1 μl 10X Buffer, 0.4 μl 25X dNTPs, 1 μl 10X Random primers, 0.5 μl Reverse Transcriptase, and 2.1 μl of water per reaction was added to 5 μl total RNA solution that was isolated using the above protocol. Plates were sealed, mixed, and incubated on a thermal cycler for 10 minutes at 25° C., followed by 2 hours at 37° C. and 5 minutes at 85° C.

Gene expression was analyzed in qPCR with Luna® Universal Probe RT-qPCR Kit (NEB) and Taqman probes-Cdh5 (Mm00486938_m1), and Gusb (Mm01197698_m1) or B2m (Mm00437762_m1). Samples were amplified using a LightCycler 480 qPCR machine (Roche). Cdh5 expression was normalized to B2m or Gusb.

### Example 1: 20% EDPOC LNP Vs. Conventional Liver Targeted LNP

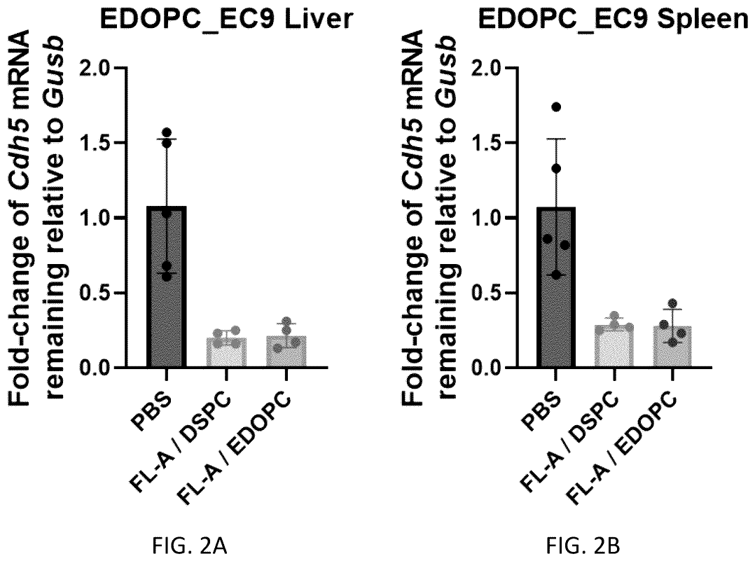

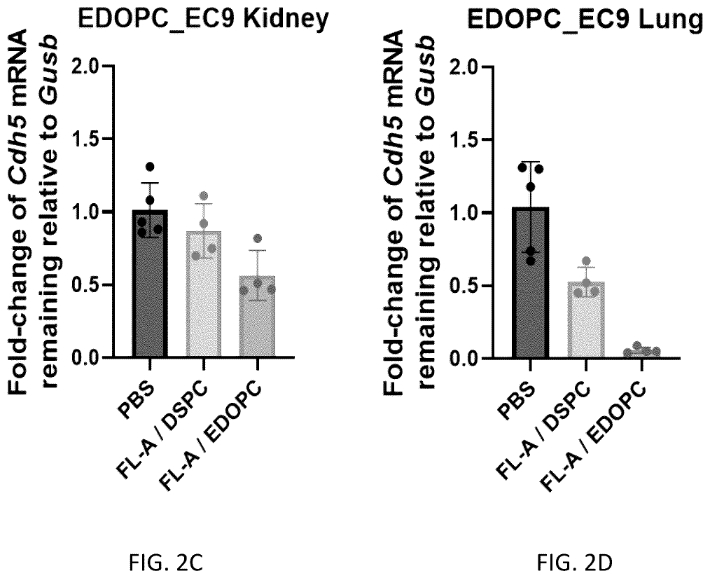

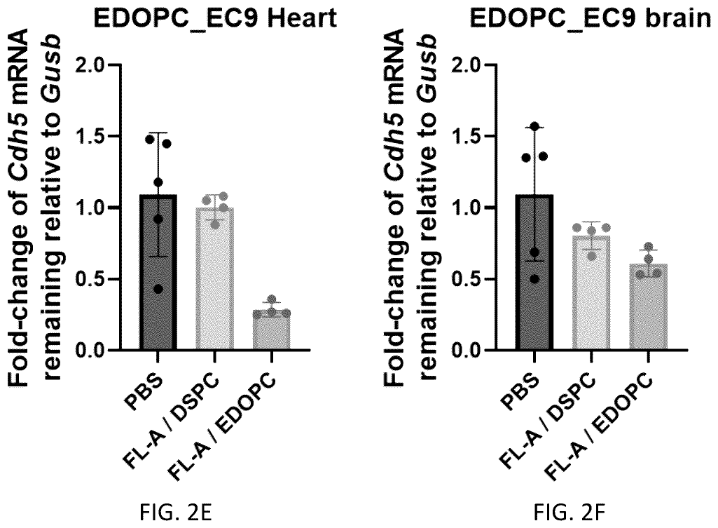

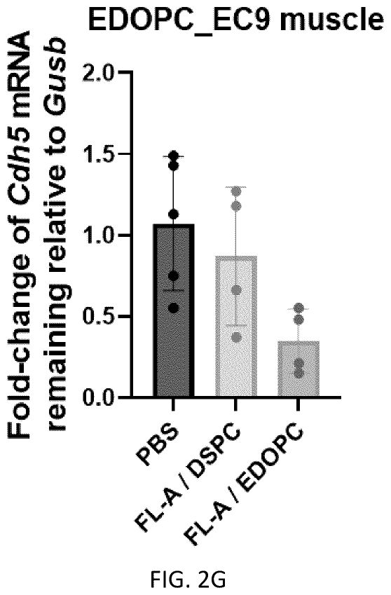

To examine the potential of cationic lipids for extrahepatic RNA delivery, DSPC was first replaced with EDOPC in a standard hepatocyte-targeting LNP formulation. The resulting LNP was formulated with siRNA against Cdh5 to evaluate gene silencing in endothelial cells in vivo. The details of the formulations are given in Table 1. The physicochemical properties of the LNPs are given in Table 2. The results are shown in FIG. 2A-2G. These results indicate that replacement of the zwitterionic phospholipid in liver-targeting LNPs with a cationic lipid can change the tissue- and cell-type tropism of LNPs.

TABLE 1
Formulations

| | | | | | | | Total |
|---|---|---|---|---|---|---|---|
| | Ionizable | Cationic | Phospho- | | | | Lipid/RNA |
| | lipid | lipid | lipid | Sterol | PEG-lipid | Molar ratio | wt ratio |
| LNP1 | FL-A | — | DSPC | Cholesterol | DMG-mPEG2000 | 50:0:10:38.5:1.5 | 16.7 |
| LNP2 | FL-A | EDOPC | — | Cholesterol | DMG-mPEG2000 | 50:20:0:28.5:1.5 | 18.0 |

TABLE 2
Physicochemical properties

| | Encapsulation | | |
|---|---|---|---|
| | Efficiency (% EE) | Z-average (nm) | PDI |
| LNP1 | 97 | 113.9 | 0.06 |
| LNP2 | 98 | 83.7 | 0.09 |

### Example 2: 20% EDOPC LNP vs. 50% DOTAP LNP

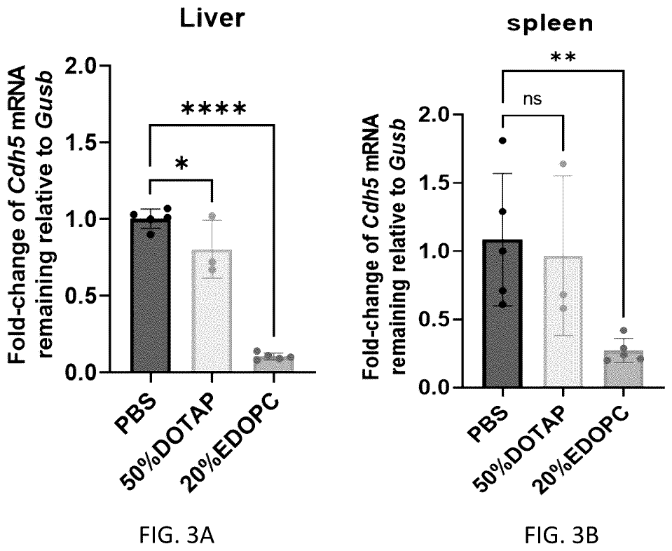

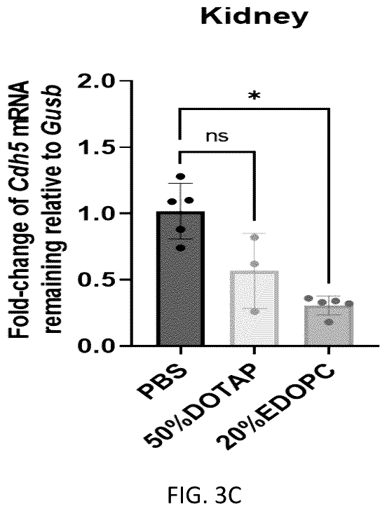

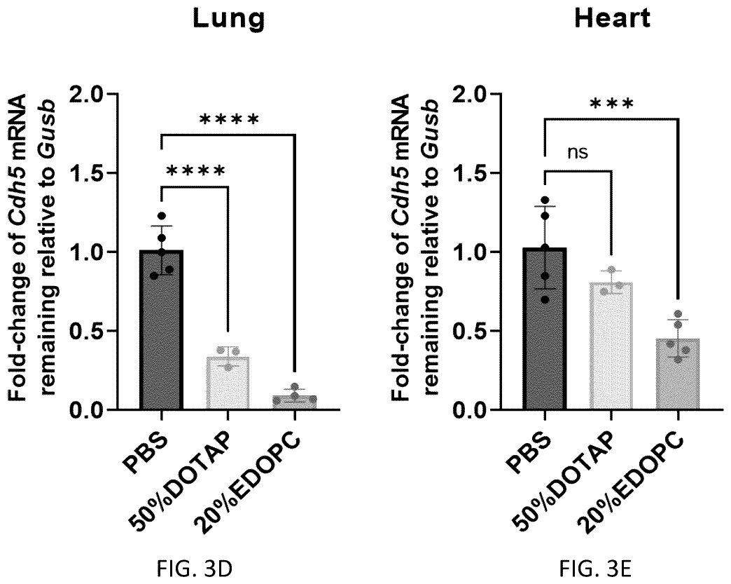

Although it has been reported that incorporation of 33-50% of DOTAP into liver-targeting LNPs enables lung RNA delivery (Q. Cheng, Nat. Nanotechnol., 2020, K. J. Kauffman, Mol. Ther.-Nucleic Acids 2018., R. J. Dorkin, MIT Thesis, 2016), RNA delivery to endothelial cells in organs in addition to the liver, spleen and lung hasn't been reported using LNPs. To explore this possibility, LNPs were formulated using 20% EDOPC and compared against previously reported LNPs with 50% DOTAP. The details of the formulations are given in Table 3. The physicochemical properties of the LNPs are given in Table 4. The results are shown in FIG. 3A-3E.

TABLE 3
Formulations

| | | | | | | | Total |
|---|---|---|---|---|---|---|---|
| | Ionizable | Cationic | Phospho- | | | | Lipid/RNA |
| | lipid | lipid | lipid | Sterol | PEG-lipid | Molar ratio | wt ratio |
| LNP3 | FL-A | DOTAP | DSPC | Cholesterol | DMG-mPEG2000 | 25:50:5:18.5:1.5 | 17.5 |
| LNP4 | FL-A | EDOPC | — | Cholesterol | DMG-mPEG2000 | 50:20:0:28.5:1.5 | 17.5 |

TABLE 4
Physicochemical properties

| | Encapsulation | | |
|---|---|---|---|
| | Efficiency (% EE) | Z-average (nm) | PDI |
| LNP3 | 100 | 81.6 | 0.13 |
| LNP4 | 100 | 96.7 | 0.04 |

### Example 3: Cationic Lipid Screening at 20 Mol %

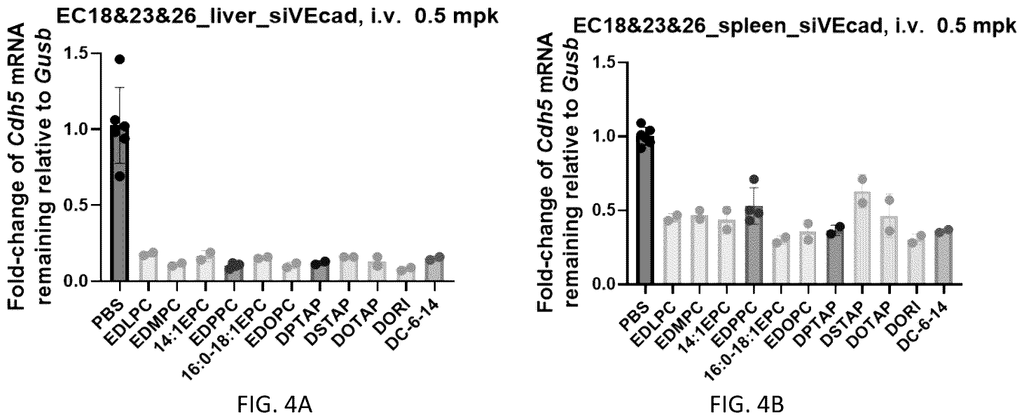

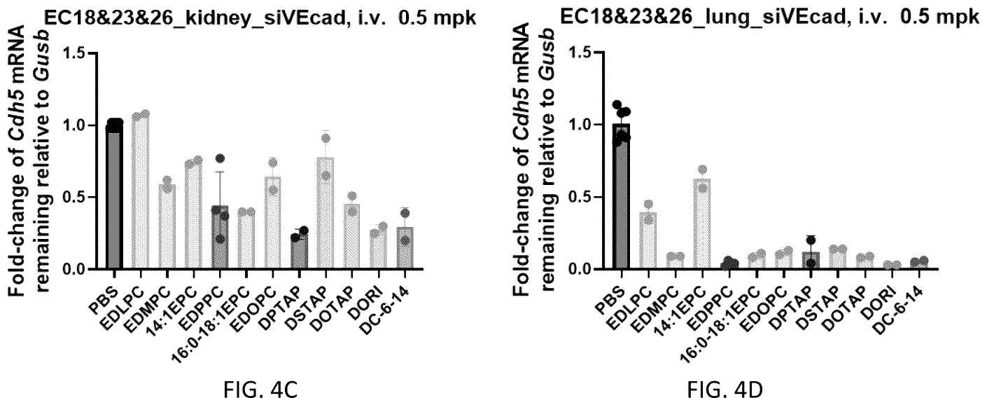

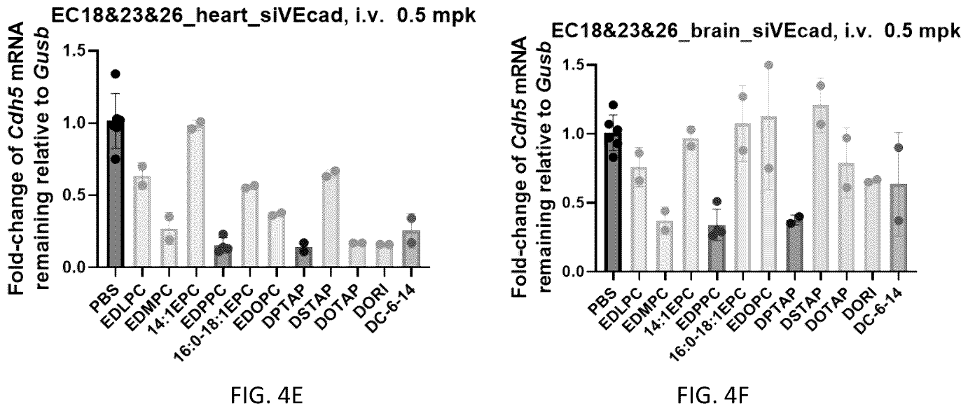

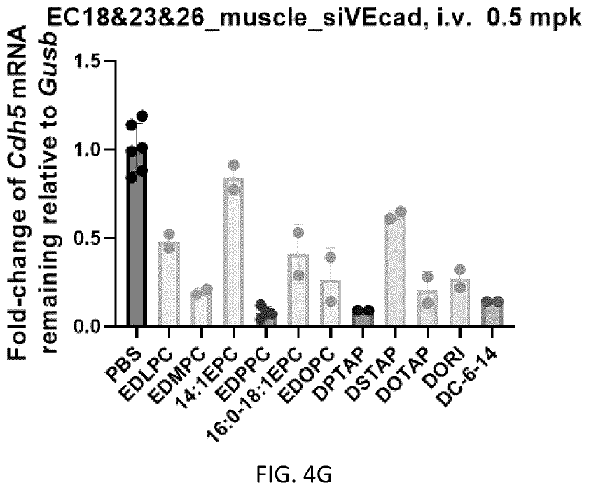

To elucidate the relationship between the chemical structure of cationic lipids and the efficiency of RNA delivery outside the liver, spleen, and lung, multiple biodegradable cationic lipids were evaluated. Three different classes of cationic lipids were prepared: Class 1 consists of cationic phospholipids, Class 2 consists of trimethylammonium-propane (TAP) lipids and DORI which is an analogue of DOTAP with ethanolamine head group, and Class 3 consists of DC-6-14. These lipids all contain hydrophilic quaternary amine head groups and biodegradable ester bonds in their hydrophobic tails but differ in their tail length, degree of saturation, and linker structure between their head and tails. LNPs containing 20% of each cationic lipid were formulated and characterized. All the cationic lipids gave pKa values of 6-7 and nearly zero to slightly positive zeta potentials. The details of the formulations are given in Table 5. The physicochemical properties of the LNPs are given in Table 6. The results are shown in FIG. 4A-4G. LNPs containing EDSPC and DMTAP were excluded from in vivo evaluation due to their large particle size (>150 nm) and inability to be filter-sterilized.

RNA delivery to endothelial cells was then evaluated in various organs by intravenously administering siCdh5 at a dose of 0.5 mg kg 1. While all the cationic lipids showed comparable levels of endothelial gene silencing in the liver and spleen, tail structure difference provided dramatic effect on delivery efficiency in the other organs. For example, in terms of degree of unsaturation, comparing C14: 0 EPC and C14: 1 EPC, C14: 0 EPC LNP delivered siRNA to endothelial cells more efficiently in the organs such as lung, heart, brain, and skeletal muscle. On the other hand, comparing DSTAP (18:0 TAP) LNP and DOTAP (18:1 TAP) LNP, DOTAP LNP showed more efficient endothelial cell delivery in the organs such as kidney, heart, brain, and skeletal muscle. Tail chain length also showed a significant impact on endothelial delivery efficiency. For example, while DSTAP (18:0 TAP) and DPTAP (16:0 TAP) differ only by two carbon atoms in their tails, DPTAP showed significantly more efficient endothelial delivery. Similarly, while EDLPC (12:0 EPC) and EDMPC (14:0 EPC) also differ only by two carbon atoms, EDMPC exhibited more efficient endothelial delivery. Furthermore, when EDPPC (16:0 EPC) and EDOPC (18:1 EPC) are compared with 16:0-18:1 EPC, which has an asymmetric tail structure and can be regarded as an intermediate structure between EDPPC and EDOPC, 16:0-18:1 EPC did not show intermediate properties between EDPPC and EDOPC and showed similar or slightly lower delivery efficiency than EDOPC.

These results indicate that, in addition to positive charge in the head, tail structure of cationic lipids contributes significantly to their endothelial delivery efficiency. In particular, cationic lipids with saturated C14 and saturated C16 symmetric tails such as EDPPC, DPTAP, and DC-6-14 showed most efficacious delivery to endothelial cells.

TABLE 5
Formulation

| | | | | | | Total |
|---|---|---|---|---|---|---|
| | Ionizable | Cationic | | | | Lipid/RNA |
| | lipid | lipid | Sterol | PEG-lipid | Molar ratio | wt ratio |
| LNP5 | FL-A | EDLPC | Cholesterol | DMG-mPEG2000 | 50:20:28.5:1.5 | 17.1 |
| LNP6 | FL-A | EDMPC | Cholesterol | DMG-mPEG2000 | 50:20:28.5:1.5 | 17.4 |
| LNP7 | FL-A | EDPPC | Cholesterol | DMG-mPEG2000 | 50:20:28.5:1.5 | 17.7 |
| LNP8 | FL-A | 14:1 EPC | Cholesterol | DMG-mPEG2000 | 50:20:28.5:1.5 | 18.0 |
| LNP9 | FL-A | 16:0-18:1EPC | Cholesterol | DMG-mPEG2000 | 50:20:28.5:1.5 | 17.8 |
| LNP10 | FL-A | EDSPC | Cholesterol | DMG-mPEG2000 | 50:20:28.5:1.5 | 18.0 |
| LNP11 | FL-A | EDOPC | Cholesterol | DMG-mPEG2000 | 50:20:28.5:1.5 | 18.0 |
| LNP12 | FL-A | DMTAP | Cholesterol | DMG-mPEG2000 | 50:20:28.5:1.5 | 16.7 |
| LNP13 | FL-A | DPTAP | Cholesterol | DMG-mPEG2000 | 50:20:28.5:1.5 | 16.9 |
| LNP14 | FL-A | DSTAP | Cholesterol | DMG-mPEG2000 | 50:20:28.5:1.5 | 17.2 |
| LNP15 | FL-A | DOTAP | Cholesterol | DMG-mPEG2000 | 50:20:28.5:1.5 | 17.2 |
| LNP16 | FL-A | DORI | Cholesterol | DMG-mPEG2000 | 50:20:28.5:1.5 | 17.6 |
| LNP17 | FL-A | DC-6-14 | Cholesterol | DMG-mPEG2000 | 50:20:28.5:1.5 | 17.0 |

TABLE 6
Physicochemical properties

| | | Encapsulation | Z- | | | Zeta |
|---|---|---|---|---|---|---|
| | Cationic | Efficiency | average | | | Potential |
| | lipid | (% EE) | (nm) | PDI | pKa | (mV) |
| LNP5 | EDLPC | 98 | 142.2 | 0.03 | 6.81 | 7.4 |
| LNP6 | EDMPC | 85 | 101.6 | 0.08 | 6.41 | 0.7 |
| LNP7 | EDPPC | 85 | 96.0 | 0.09 | 6.57 | 2.2 |
| LNP8 | 14:1 EPC | 81 | 102.1 | 0.07 | 6.89 | 0.7 |
| LNP9 | 16:0- | 82 | 92.9 | 0.07 | 6.78 | −1.7 |
| | 18:1EPC | | | | | |
| LNP10 | EDSPC | 99 | 179.0 | 0.16 | 6.28 | 11.5 |
| LNP11 | EDOPC | 99 | 78.5 | 0.09 | 6.83 | 4.8 |
| LNP12 | DMTAP | 100 | 238.2 | 0.10 | N.D. | 10.9 |
| LNP13 | DPTAP | 96 | 120.2 | 0.01 | 6.19 | 7.6 |
| LNP14 | DSTAP | 87 | 101.6 | 0.06 | 6.57 | 6.9 |
| LNP15 | DOTAP | 100 | 84.0 | 0.10 | 6.74 | 7.6 |
| LNP16 | DORI | 97 | 74.3 | 0.06 | 7.00 | 5.0 |
| LNP17 | DC-6-14 | 93 | 112.1 | 0.05 | 6.49 | 2.2 |

### Example 4: Advantage of EDPPC and DPTAP Over EDMPC and DOTAP

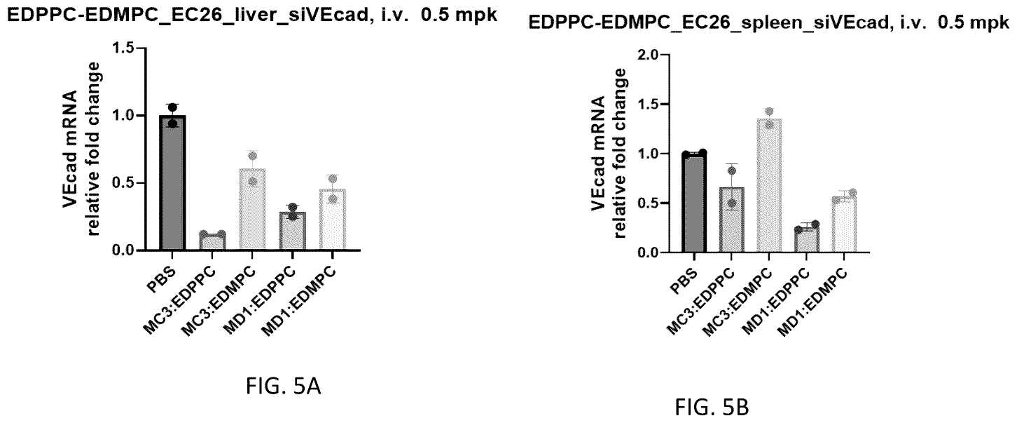

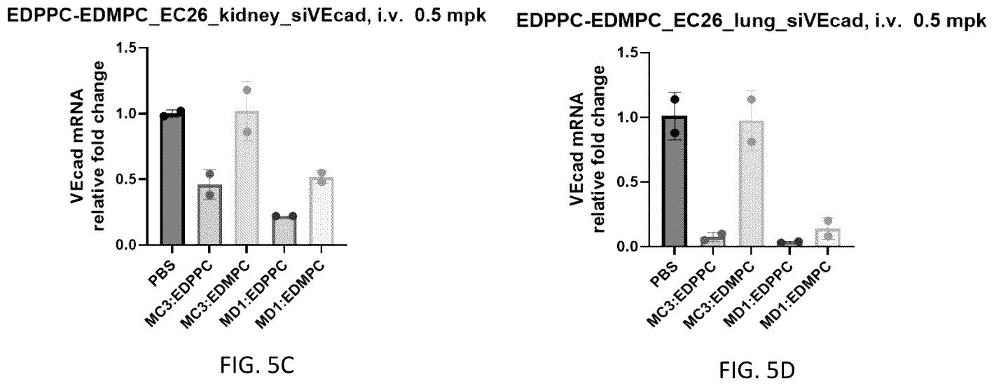

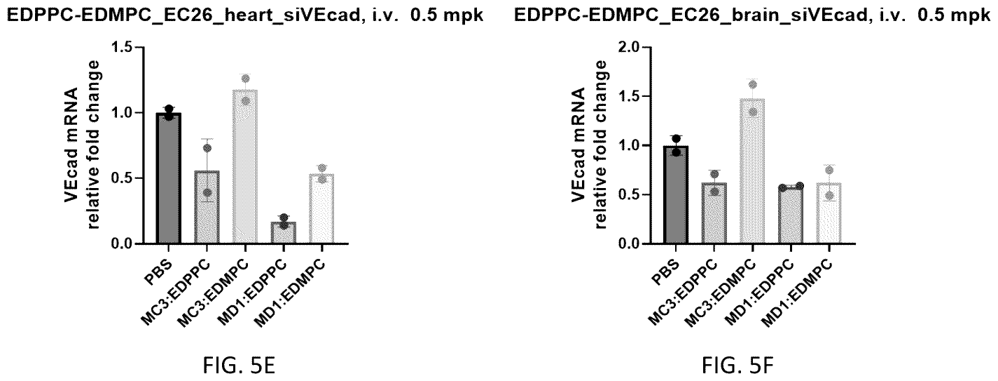

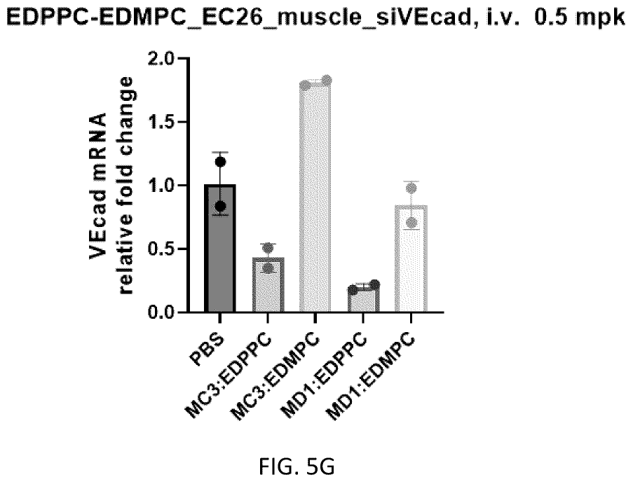

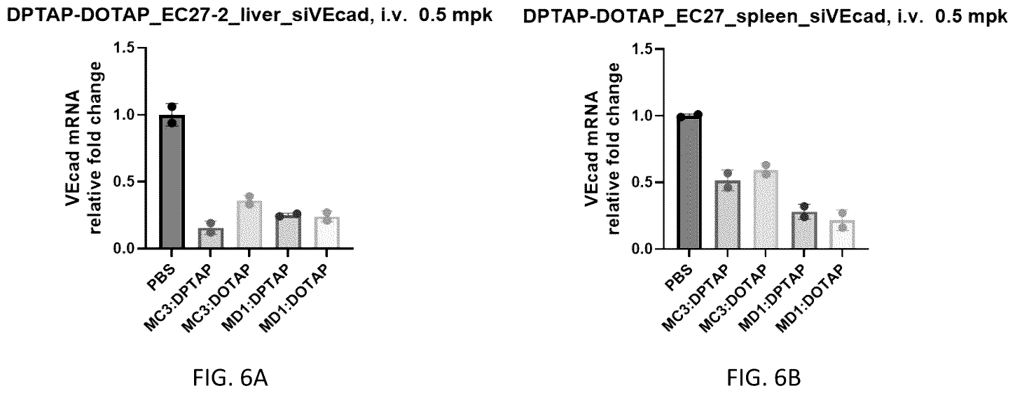

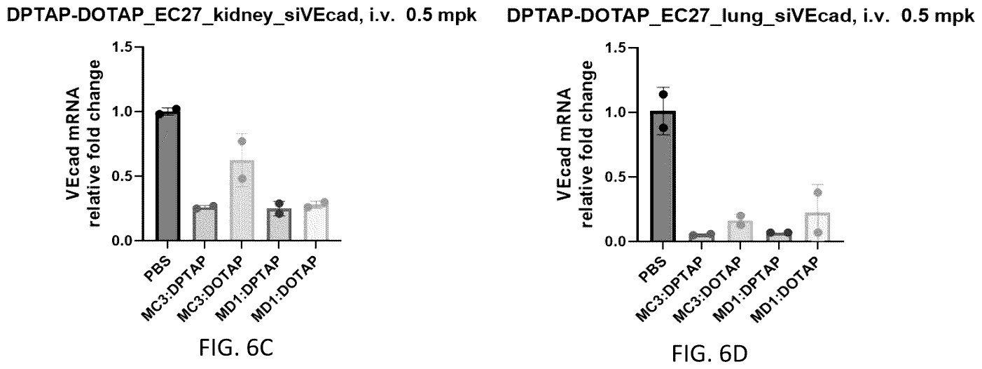

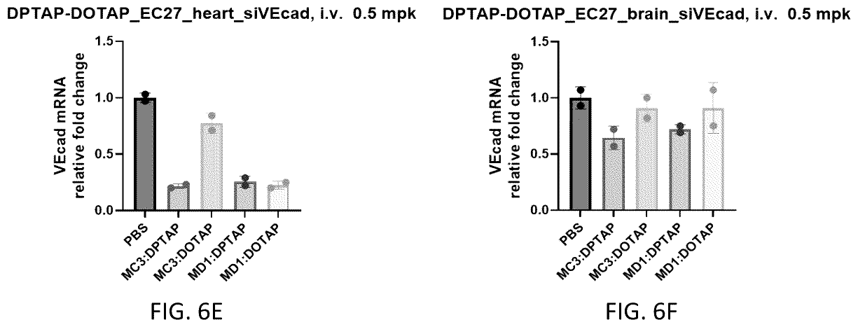

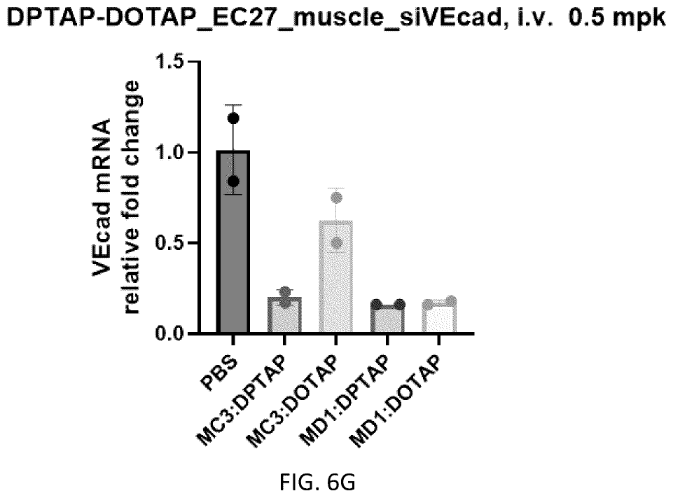

To further confirm the superiority of EDPPC over EDMPC and DPTAP over DOTAP, these cationic lipids were formulated with MC3 and MD-1 and evaluated for in vivo endothelial RNA delivery efficiency. LNPs encapsulating siRNA against Cdh5 were intravenously administered to mice at 0.5 mg/kg and organs were harvested 72 hours after injection. After isolation and purification of total RNA from the organs, Cdh5 mRNA was quantified relative to a housekeeping gene, Gusb. The details of the formulations are given in Table 7. The physicochemical properties of the LNPs are given in Table 8. The results are shown in FIG. 5A-5G and FIG. 6A-6G.

TABLE 7
Formulation

| | Ionizable | Cationic | | | | Lipid/RNA |
|---|---|---|---|---|---|---|
| | lipid | lipid | Sterol | PEG-lipid | Molar ratio | wt ratio |
| LNP18 | MC3 | EDPPC | Cholesterol | DMG-mPEG2000 | 50:20:18.5:1.5 | 21.5 |
| LNP19 | MC3 | EDMPC | Cholesterol | DMG-mPEG2000 | 50:20:18.5:1.5 | 21.6 |
| LNP20 | MC3 | DPTAP | Cholesterol | DMG-mPEG2000 | 50:20:18.5:1.5 | 20.4 |
| LNP21 | MC3 | DOTAP | Cholesterol | DMG-mPEG2000 | 50:20:18.5:1.5 | 20.5 |
| LNP22 | MD-1 | EDPPC | Cholesterol | DMG-mPEG2000 | 50:20:18.5:1.5 | 21.6 |
| LNP23 | MD-1 | EDMPC | Cholesterol | DMG-mPEG2000 | 50:20:18.5:1.5 | 21.5 |
| LNP24 | MD-1 | DPTAP | Cholesterol | DMG-mPEG2000 | 50:20:18.5:1.5 | 20.7 |
| LNP25 | MD-1 | DOTAP | Cholesterol | DMG-mPEG2000 | 50:20:18.5:1.5 | 20.7 |

TABLE 8
Physicochemical Properties

| | Ionizable | Cationic | Encapsulation | Z-average | |
|---|---|---|---|---|---|
| | lipid | lipid | Efficiency (% EE) | (nm) | PDI |
| LNP18 | MC3 | EDPPC | 94 | 80.7 | 0.10 |
| LNP19 | MC3 | EDMPC | 84 | 67.6 | 0.17 |
| LNP20 | MC3 | DPTAP | 93 | 94.2 | 0.12 |
| LNP21 | MC3 | DOTAP | 97 | 72.5 | 0.17 |
| LNP22 | MD-1 | EDPPC | 70 | 90.5 | 0.09 |
| LNP23 | MD-1 | EDMPC | 60 | 89.4 | 0.15 |
| LNP24 | MD-1 | DPTAP | 59 | 84.6 | 0.12 |
| LNP25 | MD-1 | DOTAP | 75 | 95.5 | 0.10 |

### Example 5: Combination of Ionizable Lipid and Cationic Lipid

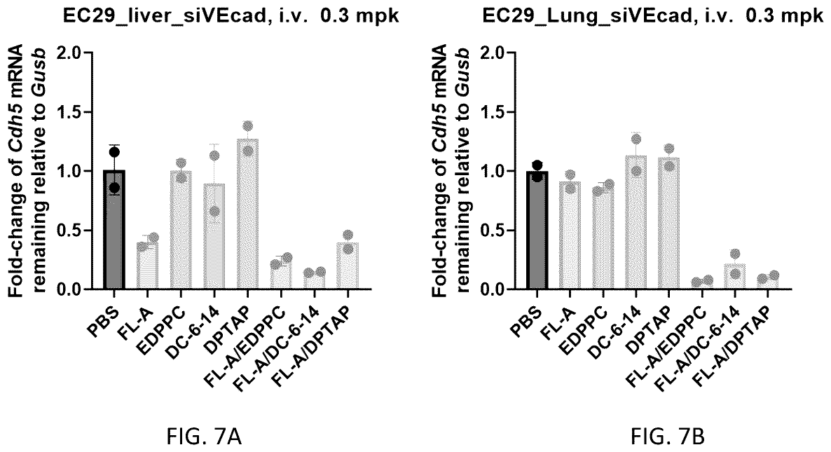

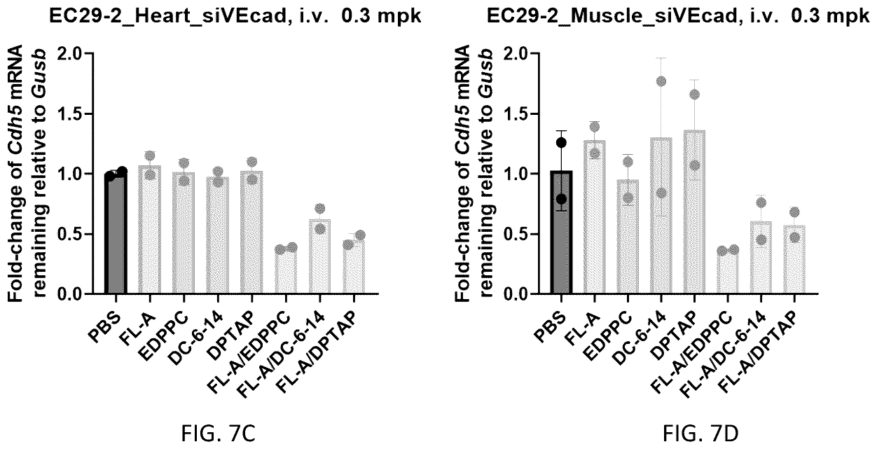

To further confirm the necessity of the combination of ionizable lipid and cationic lipid for endothelial RNA delivery, LNPs without ionizable lipid were prepared and compared with LNPs containing both an ionizable lipid and a cationic lipid. LNPs encapsulating siRNA against Cdh5 were intravenously administered to mice at 0.3 mg/kg and organs were harvested 72 hours after injection. After isolation and purification of total RNA from the organs, Cdh5 mRNA was quantified relative to a housekeeping gene, Gusb. The details of the formulations are given in Table 9. The physicochemical properties of the LNPs are given in Table 10. The results are shown in FIG. 7A-7D.

TABLE 9
Formulation

| | Ionizable | Cationic | Phospho- | | | | Lipid/RNA |
|---|---|---|---|---|---|---|---|
| | lipid | lipid | lipid | Cholesterol | PEG-lipid | Molar ratio | wt ratio |
| LNP26 | FL-A | — | DSPC | Cholesterol | DMG-mPEG2000 | 50:0:10:38.5:1.5 | 20.8 |
| LNP27 | — | EDPPC | — | Cholesterol | DMG-mPEG2000 | 0:50:0.38:2 | 21.6 |
| LNP28 | — | DC-6-14 | — | Cholesterol | DMG-mPEG2000 | 0:50:0.38:2 | 21.6 |
| LNP29 | — | DPTAP | — | Cholesterol | DMG-mPEG2000 | 0:50:0.38:2 | 17.6 |
| LNP30 | FL-A | EDPPC | — | Cholesterol | DMG-mPEG2000 | 39:20:0:39:2 | 16.9 |
| LNP31 | FL-A | DC-6-14 | — | Cholesterol | DMG-mPEG2000 | 39:20:0:39:2 | 18.4 |
| LNP32 | FL-A | DPTAP | — | Cholesterol | DMG-mPEG2000 | 39:20:0:39:2 | 18.6 |

TABLE 10
Physicochemical Properties

| | Encapsulation | Z-average | | | Zeta Potential |
|---|---|---|---|---|---|
| | Efficiency (% EE) | (nm) | PDI | pKa | (mV) |
| LNP26 | 87 | 95.1 | 0.06 | 6.86 | −3.7 |
| LNP27 | 98 | 119.5 | 0.17 | >8.5 | 7.4 |
| LNP28 | 96 | 114.9 | 0.11 | >8.5 | 7.1 |
| LNP29 | 97 | 143.6 | 0.15 | >8.5 | 7.3 |
| LNP30 | 82 | 103.5 | 0.08 | 6.31 | 10.3 |
| LNP31 | 83 | 98.5 | 0.07 | 6.49 | 7.5 |
| LNP32 | 88 | 100.3 | 0.10 | 6.19 | 8.1 |

### Example 6: Generalization of Ionizable Lipids (20% EDPPC)

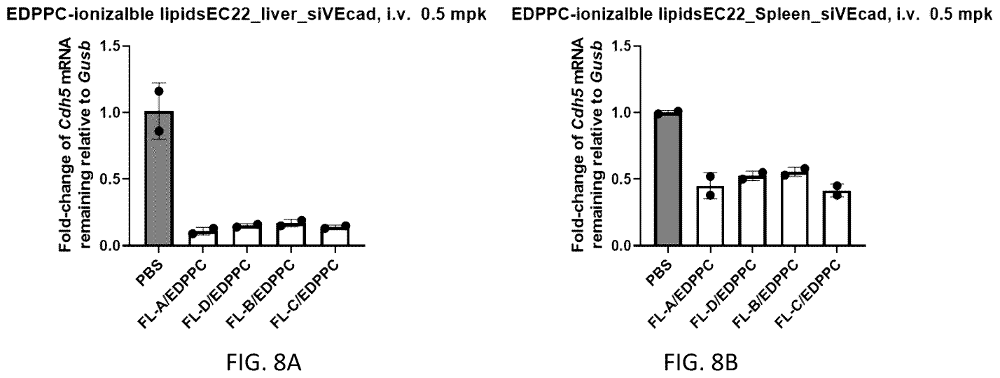

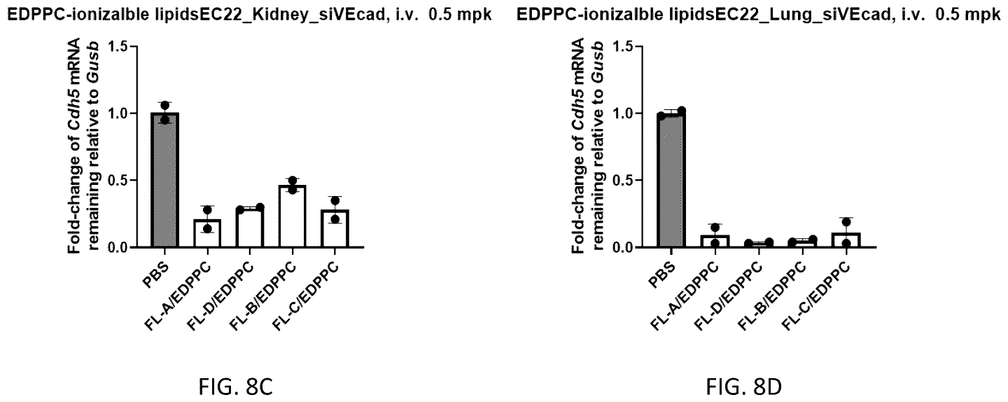

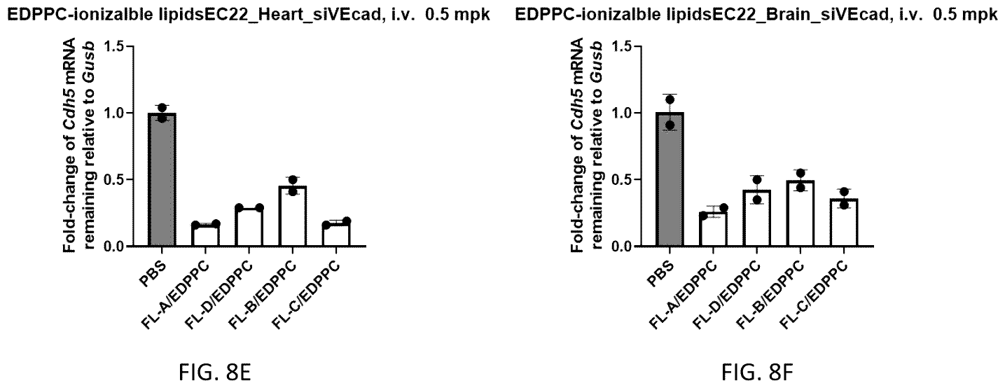

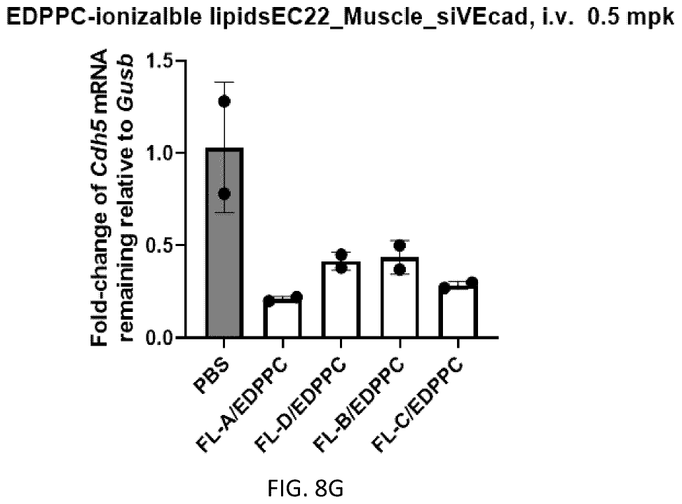

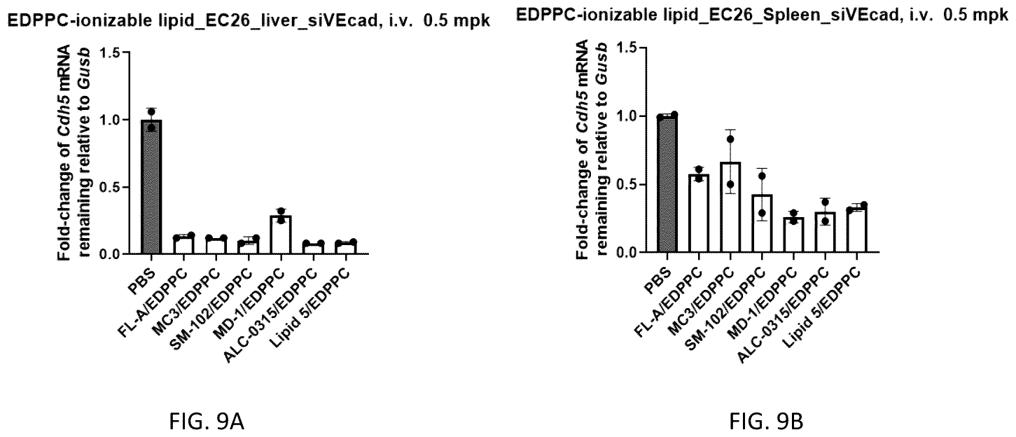

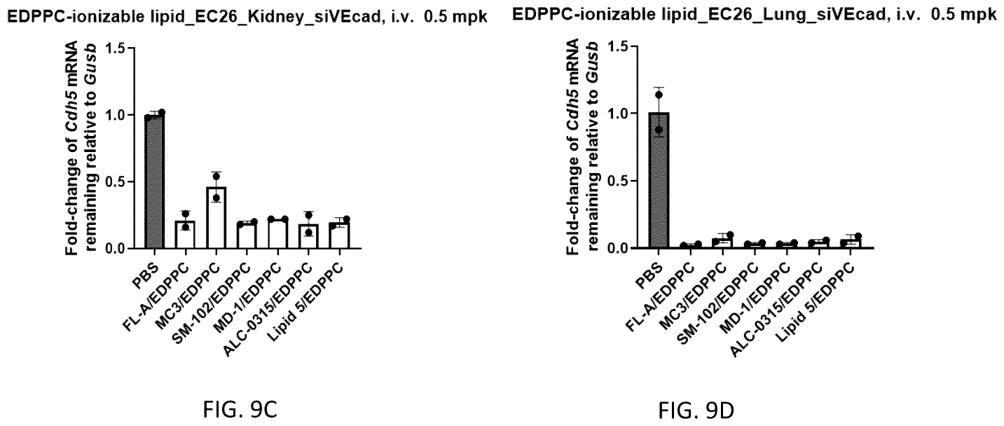

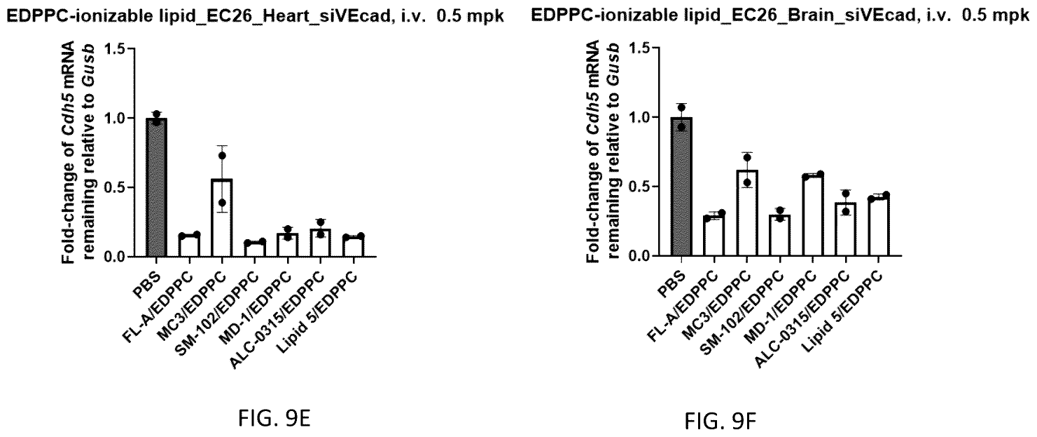

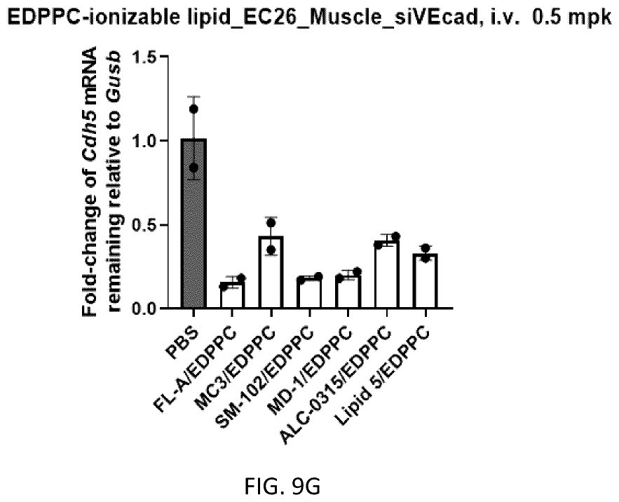

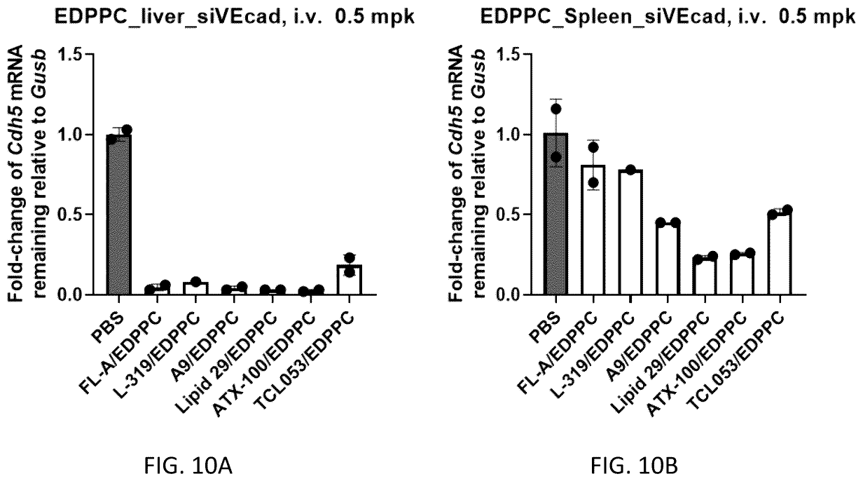

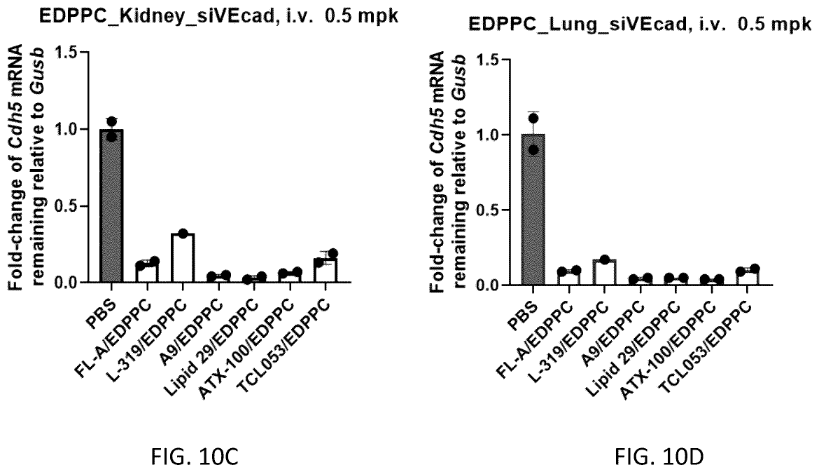

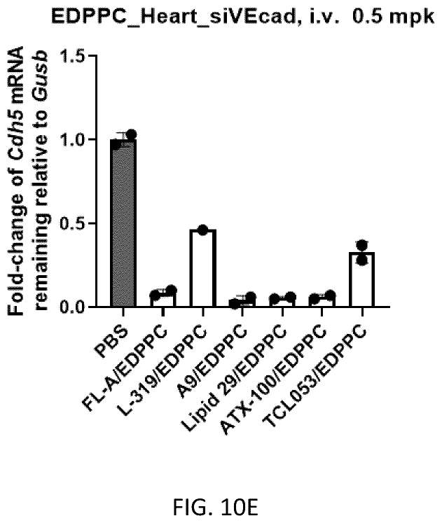

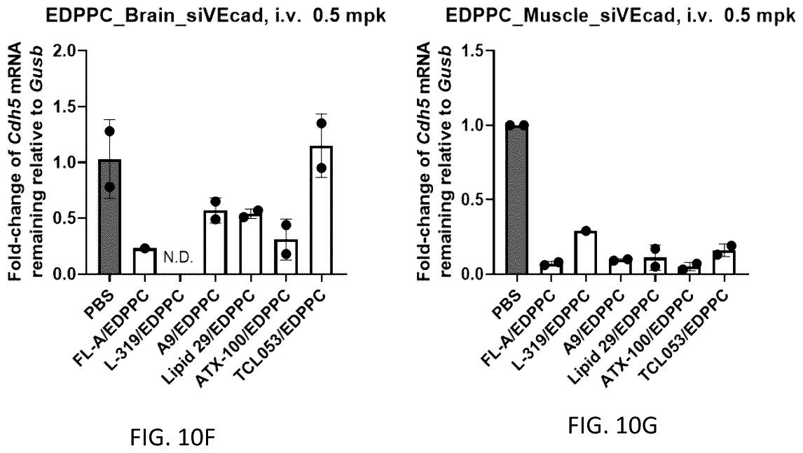

To examine the generalizability of these cationic lipids for extrahepatic delivery, various ionizable lipids were formulated into LNPs containing 20% EDPPC, 20% DPTAP, and 20% DC-6-14. First, LNPs containing 20% EDPPC with 15 different ionizable lipids were prepared. Surprisingly, all LNPs showed efficacious Cdh5 gene silencing on endothelial cells in all the tested organs with single 0.5 mg/kg siCdh5 administration. The details of the formulations are given in Table 11. The physicochemical properties of the LNPs are given in Table 12. The results are shown in FIG. 8A-8G, FIG. 9A-9G, and FIG. 10A-10G.

TABLE 11
Formulations

| | Ionizable | Cationic | | | | Lipid/RNA |
|---|---|---|---|---|---|---|
| | lipid | lipid | Sterol | PEG-lipid | Molar ratio | wt ratio |
| LNP33 | FL-D | EDPPC | Cholesterol | DMG-mPEG2000 | 39.25:20:39.25:1.5 | 15.3 |
| LNP34 | FL-B | EDPPC | Cholesterol | DMG-mPEG2000 | 39.25:20:39.25:1.5 | 15.7 |
| LNP35 | FL-A | EDPPC | Cholesterol | DMG-mPEG2000 | 39.25:20:39.25:1.5 | 16.9 |
| LNP36 | FL-C | EDPPC | Cholesterol | DMG-mPEG2000 | 39.25:20:39.25:1.5 | 18.0 |
| LNP37 | FL-A | EDPPC | Cholesterol | DMG-mPEG2000 | 39:20:39:2 | 21.6 |
| LNP38 | MC3 | EDPPC | Cholesterol | DMG-mPEG2000 | 39:20:39:2 | 21.5 |
| LNP39 | SM-102 | EDPPC | Cholesterol | DMG-mPEG2000 | 39:20:39:2 | 21.6 |
| LNP40 | MD-1 | EDPPC | Cholesterol | DMG-mPEG2000 | 39:20:39:2 | 21.6 |
| LNP41 | ALC-0315 | EDPPC | Cholesterol | DMG-mPEG2000 | 39:20:39:2 | 21.6 |
| LNP42 | Lipid5 | EDPPC | Cholesterol | DMG-mPEG2000 | 39:20:39:2 | 21.6 |
| LNP91 | FL-A | EDPPC | Cholesterol | DMG-mPEG2000 | 39:20:39:2 | 21.6 |
| LNP92 | L-319 | EDPPC | Cholesterol | DMG-mPEG2000 | 39:20:39:2 | 23.6 |
| LNP93 | A9 | EDPPC | Cholesterol | DMG-mPEG2000 | 39:20:39:2 | 20.2 |
| LNP94 | Lipid 29 | EDPPC | Cholesterol | DMG-mPEG2000 | 39:20:39:2 | 20.7 |
| LNP95 | ATX-100 | EDPPC | Cholesterol | DMG-mPEG2000 | 39:20:39:2 | 22.2 |
| LNP96 | Lp01 | EDPPC | Cholesterol | DMG-mPEG2000 | 39:20:39:2 | 20.8 |
| LNP97 | TCL053 | EDPPC | Cholesterol | DMG-mPEG2000 | 39:20:39:2 | 20.6 |

TABLE 12
Physicochemical Properties

| | Ionizable | Encapsulation | Z-average | | |
|---|---|---|---|---|---|
| | lipid | Efficiency (% EE) | (nm) | PDI | pKa |
| LNP33 | FL-D | 97 | 86.0 | 0.07 | 6.56 |
| LNP34 | FL-B | 96 | 87.3 | 0.09 | 6.45 |
| LNP35 | FL-A | 95 | 90.7 | 0.05 | 6.38 |
| LNP36 | FL-C | 96 | 94.9 | 0.08 | 6.69 |
| LNP37 | FL-A | 95 | 112.3 | 0.09 | 6.32 |
| LNP38 | MC3 | 94 | 80.7 | 0.10 | 6.37 |
| LNP39 | SM-102 | 90 | 87.8 | 0.10 | 6.79 |
| LNP40 | MD-1 | 70 | 90.5 | 0.09 | 5.27 |
| LNP41 | ALC-0315 | 87 | 89.2 | 0.08 | 6.48 |
| LNP42 | Lipid5 | 86 | 85.3 | 0.11 | 6.75 |
| LNP91 | FL-A | 84 | 133.1 | 0.07 | 6.47 |
| LNP92 | L-319 | 90 | 173.3 | 0.06 | 6.42 |
| LNP93 | A9 | 91 | 94.0 | 0.08 | 6.68 |
| LNP94 | Lipid 29 | 83 | 81.2 | 0.10 | 5.22 |
| LNP95 | ATX-100 | 83 | 96.3 | 0.11 | 6.17 |
| LNP96 | Lp01 | 78 | 105.3 | 0.05 | 6.45 |
| LNP97 | TCL053 | 86 | 122.4 | 0.15 | 5.54 |

### Example 7: Generalization of Ionizable Lipids (20% DPTAP)

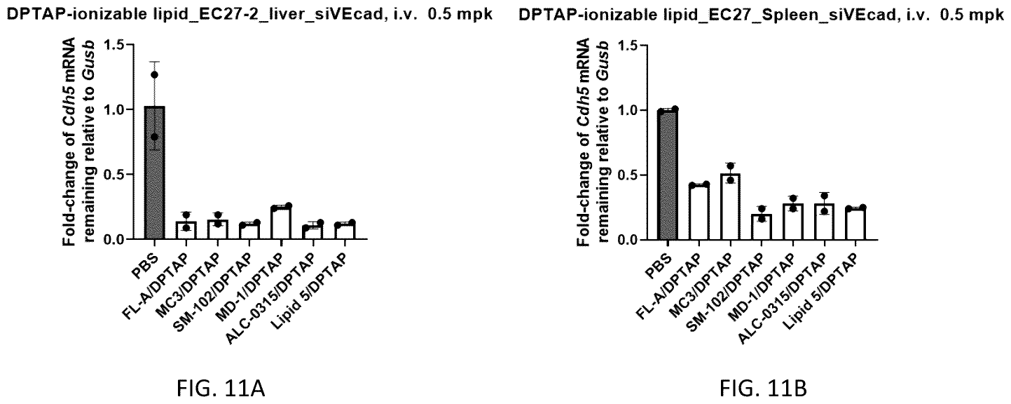

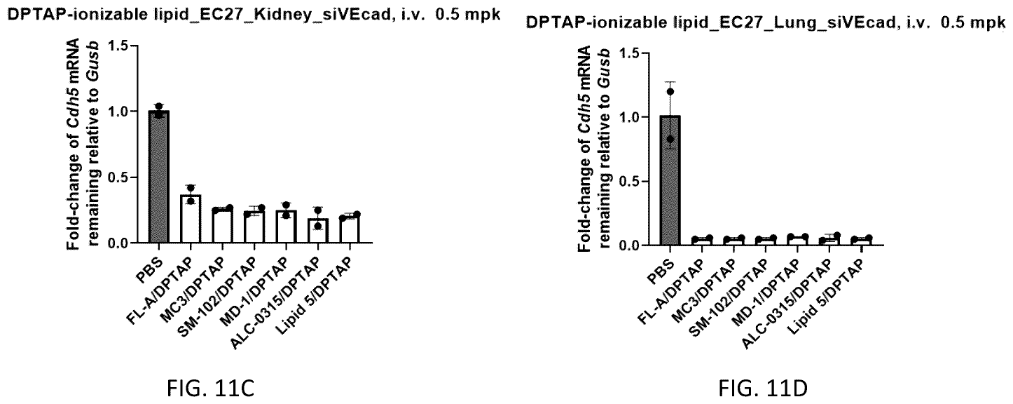

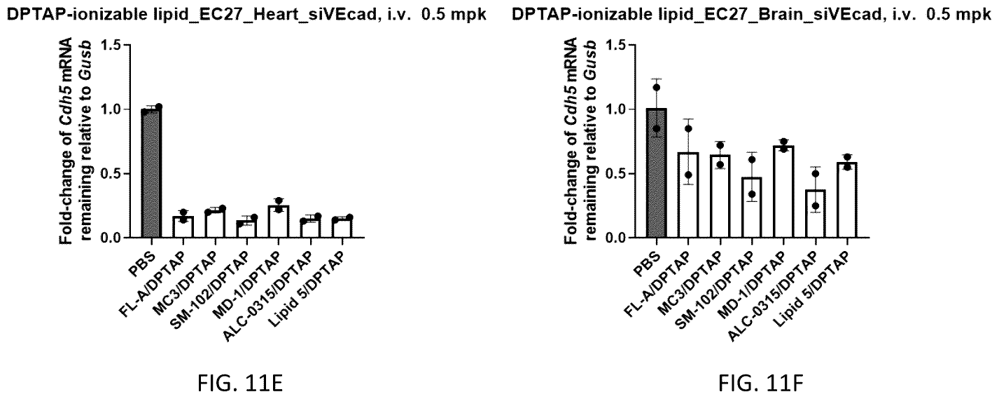

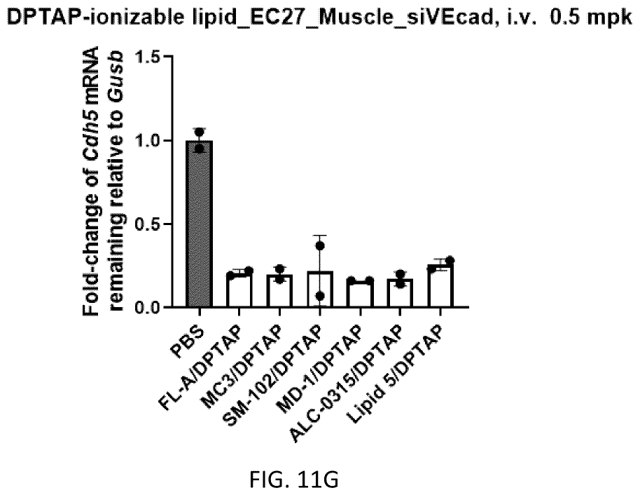

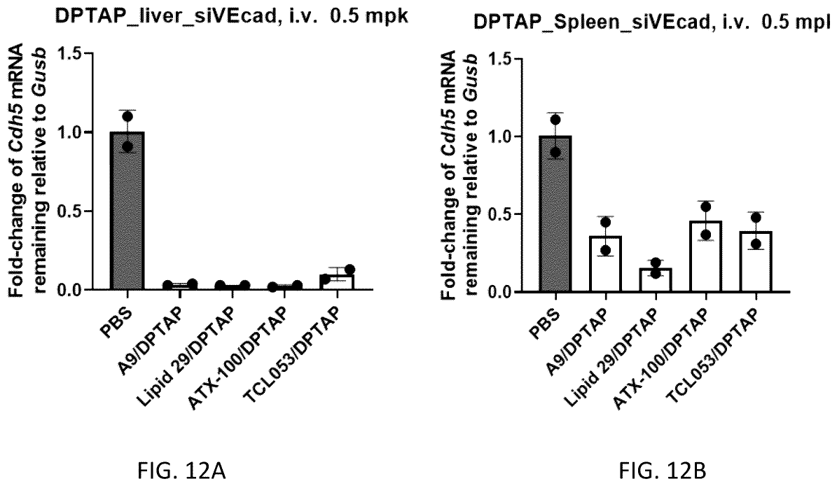

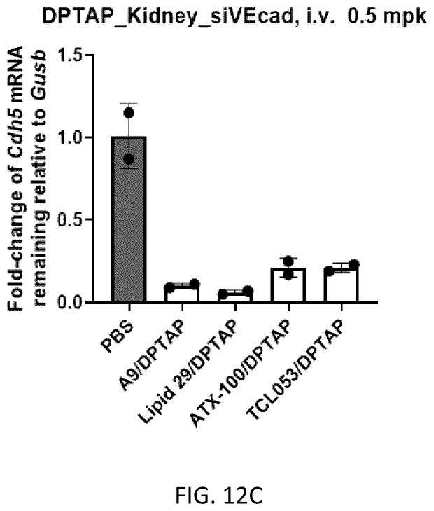

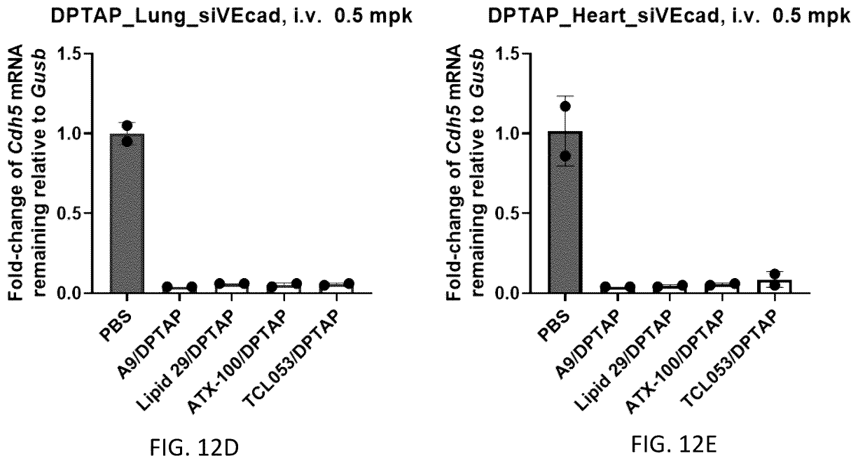

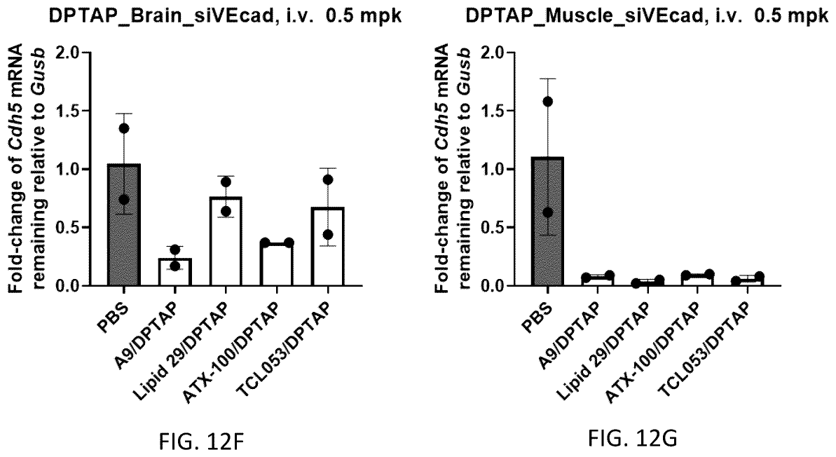

To continue examining the generalizability of these cationic lipids for extrahepatic delivery, various ionizable lipids were formulated into LNPs containing 20% EDPPC, 20% DPTAP, and 20% DC-6-14. LNPs were prepared containing 20% DPTAP with 11 different ionizable lipids. Surprisingly, all LNPs showed efficacious Cdh5 gene silencing on endothelial cells in all the tested organs with a single 0.5 mg/kg siCdh5 administration. The details of the formulations are given in Table 13. The physicochemical properties of the LNPs are given in Table 14. The results are shown in FIG. 11A-11G and FIG. 12A-12G.

TABLE 13
Formulations

| | Ionizable | Cationic | | | | Lipid/RNA |
|---|---|---|---|---|---|---|
| | lipid | lipid | Cholesterol | PEG-lipid | Molar ratio | wt ratio |
| LNP43 | FL-A | DPTAP | Cholesterol | DMG-mPEG2000 | 39:20:39:2 | 20.6 |
| LNP44 | MC3 | DPTAP | Cholesterol | DMG-mPEG2000 | 39:20:39:2 | 20.4 |
| LNP45 | SM-102 | DPTAP | Cholesterol | DMG-mPEG2000 | 39:20:39:2 | 20.6 |
| LNP46 | MD-1 | DPTAP | Cholesterol | DMG-mPEG2000 | 39:20:39:2 | 20.7 |
| LNP47 | ALC-0315 | DPTAP | Cholesterol | DMG-mPEG2000 | 39:20:39:2 | 20.6 |
| LNP48 | Lipid5 | DPTAP | Cholesterol | DMG-mPEG2000 | 39:20:39:2 | 20.6 |
| LNP98 | A9 | DPTAP | Cholesterol | DMG-mPEG2000 | 39:20:39:2 | 19.3 |
| LNP99 | Lipid 29 | DPTAP | Cholesterol | DMG-mPEG2000 | 39:20:39:2 | 19.8 |
| LNP100 | ATX-100 | DPTAP | Cholesterol | DMG-mPEG2000 | 39:20:39:2 | 21.2 |
| LNP101 | Lp01 | DPTAP | Cholesterol | DMG-mPEG2000 | 39:20:39:2 | 19.9 |
| LNP102 | TCL053 | DPTAP | Cholesterol | DMG-mPEG2000 | 39:20:39:2 | 19.7 |

TABLE 14
Physicochemical properties

| | Ionizable | Encapsulation | | | |
|---|---|---|---|---|---|
| | lipid | Efficiency (% EE) | Z-average (nm) | PDI | pKa |
| LNP43 | FL-A | 89 | 102.4 | 0.10 | 6.27 |
| LNP44 | MC3 | 93 | 94.2 | 0.12 | 5.97 |
| LNP45 | SM-102 | 89 | 96.0 | 0.18 | 6.70 |
| LNP46 | MD-1 | 59 | 84.6 | 0.12 | 5.27 |
| LNP47 | ALC-0315 | 86 | 86.3 | 0.11 | 6.36 |
| LNP48 | Lipid5 | 89 | 81.5 | 0.16 | 6.47 |
| LNP98 | A9 | 93 | 84.0 | 0.05 | 6.65 |
| LNP99 | Lipid 29 | 93 | 74.9 | 0.07 | 4.94 |
| LNP100 | ATX-100 | 94 | 119.3 | 0.04 | 5.19 |
| LNP101 | Lp01 | 87 | 138.8 | 0.08 | 6.22 |
| LNP102 | TCL053 | 91 | 95.6 | 0.15 | 6.34 |

### Example 8: Generalization of Ionizable Lipids (20% DC-6-14)

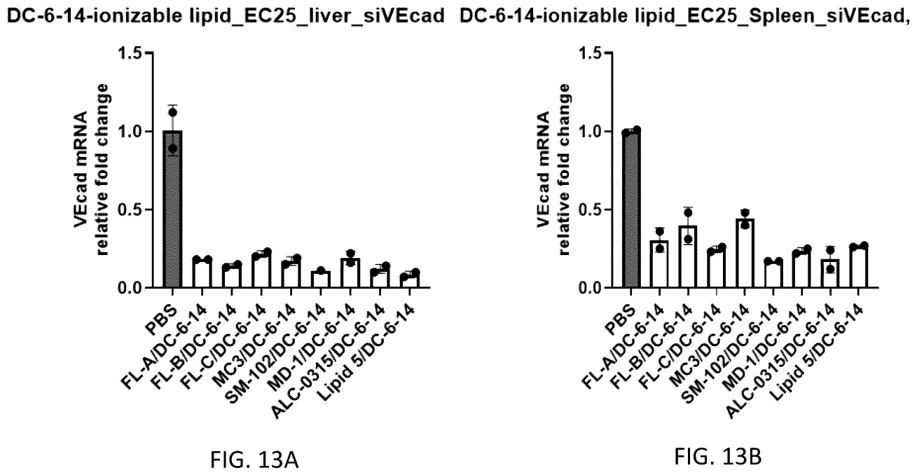

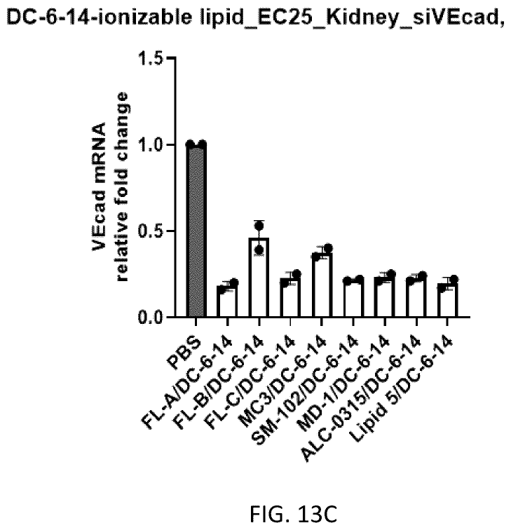

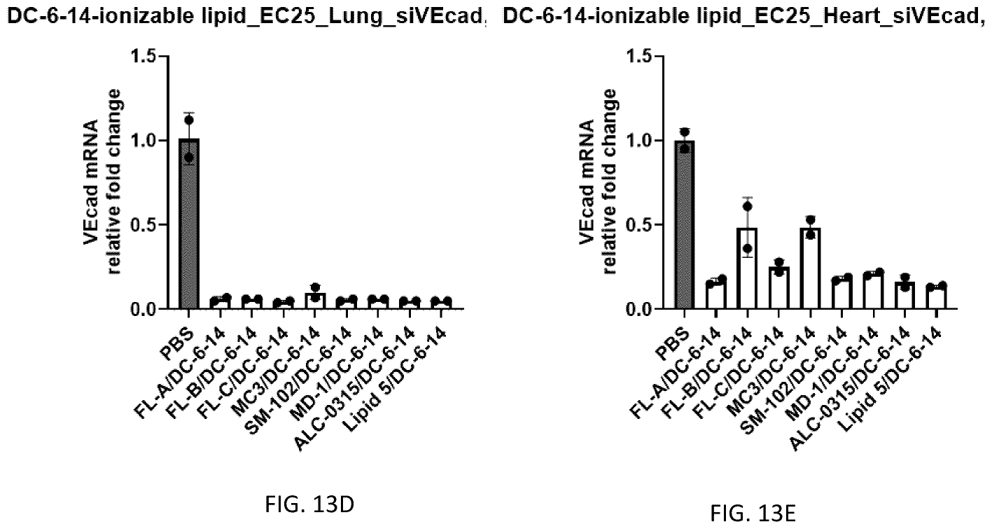

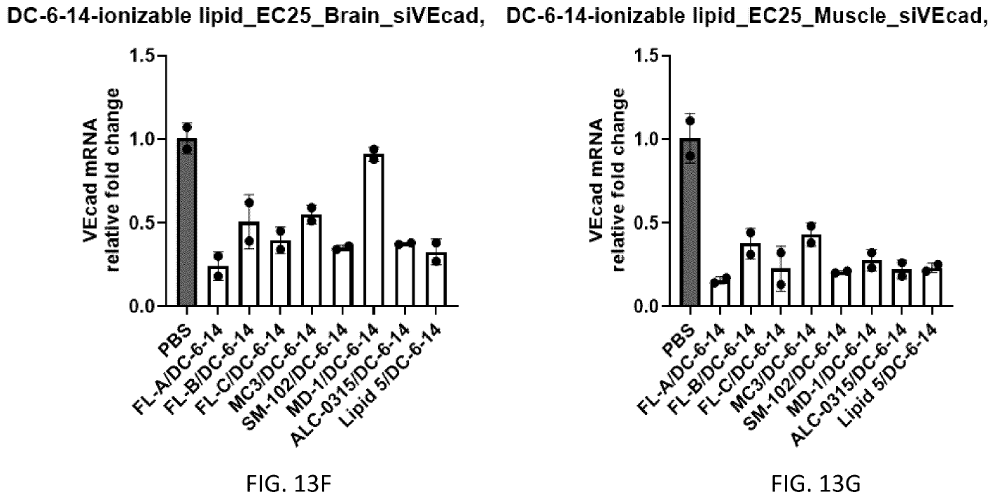

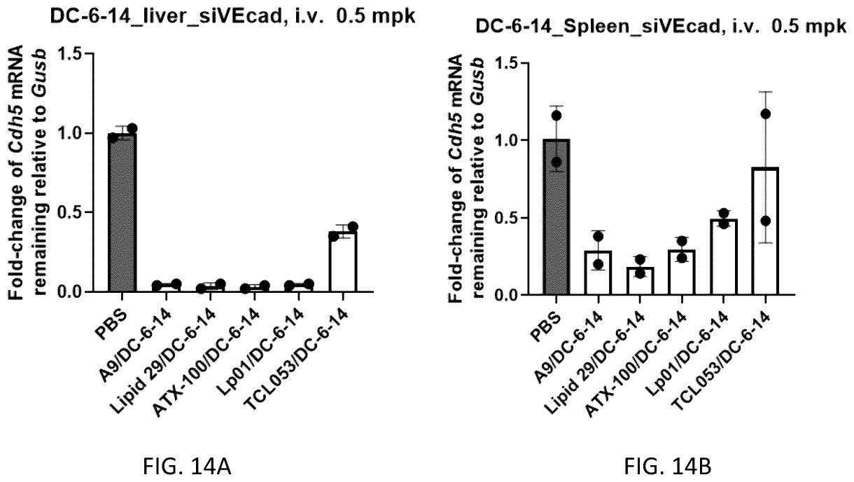

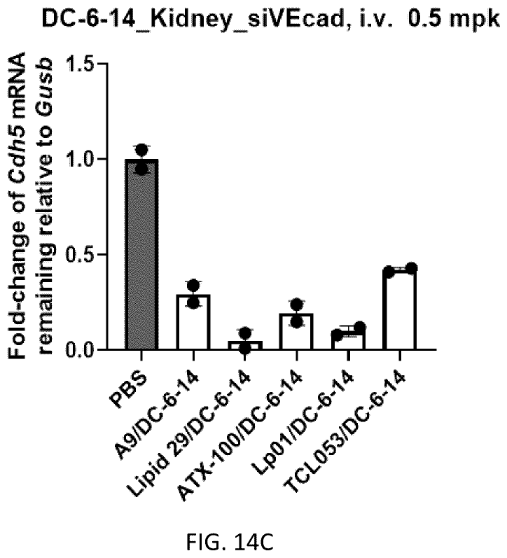

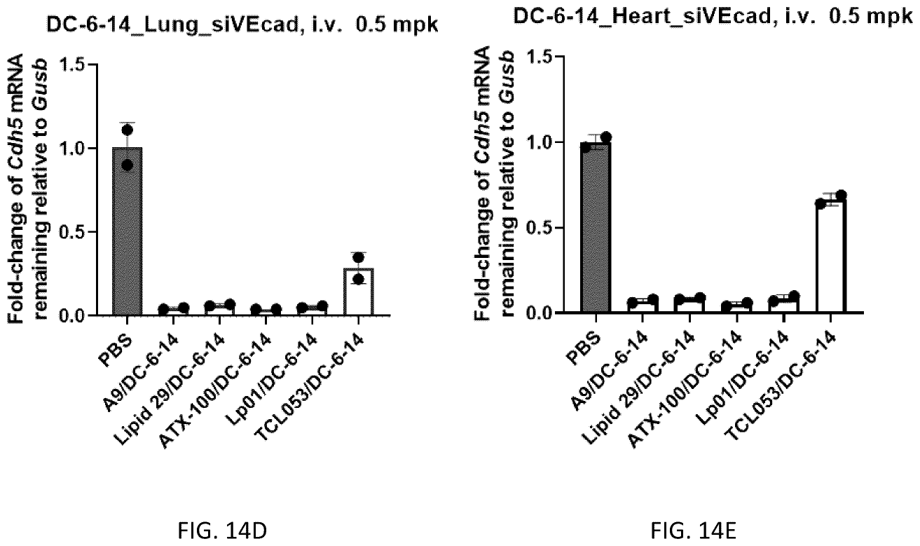

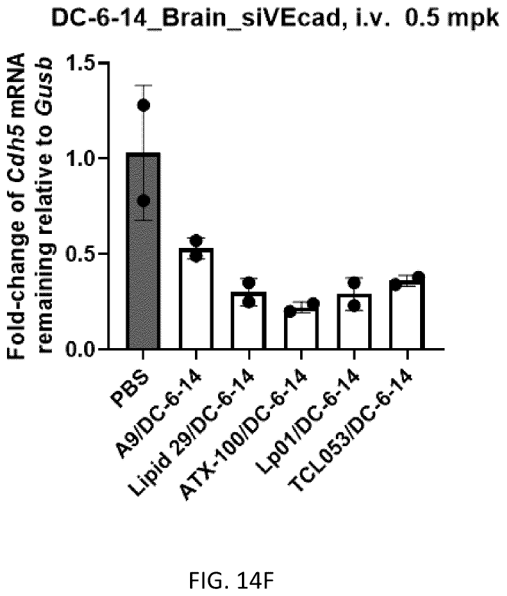

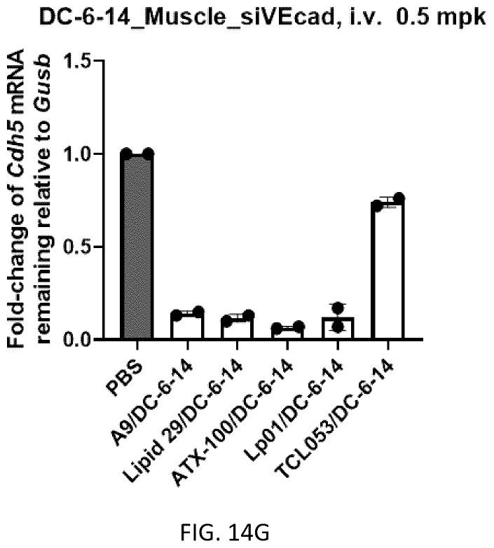

To continue examining the generalizability of these cationic lipids for extrahepatic delivery, various ionizable lipids were formulated into LNPs containing 20% EDPPC, 20% DPTAP, and 20% DC-6-14. Finally, the generalizability of DC-6-14 was further examined. LNPs containing 20% DC-6-14 with 13 different ionizable lipids were further examined. Surprisingly, all LNPs showed efficacious Cdh5 gene silencing on endothelial cells in all the tested organs with a single 0.5 mg/kg siCdh5 administration. The details of the formulations are given in Table 15. The physicochemical properties of the LNPs are given in Table 16. The results are shown in FIG. 13A-13G and FIG. 14A-14G.

TABLE 15
Formulation

| | Ionizable | Cationic | | | | Lipid/RNA |
|---|---|---|---|---|---|---|
| | lipid | lipid | Sterol | PEG-lipid | Molar ratio | wt ratio |
| LNP49 | FL-A | DC-6-14 | Cholesterol | DMG-mPEG2000 | 39:20:39:2 | 20.7 |
| LNP50 | FL-B | DC-6-14 | Cholesterol | DMG-mPEG2000 | 39:20:39:2 | 20.6 |
| LNP51 | FL-C | DC-6-14 | Cholesterol | DMG-mPEG2000 | 39:20:39:2 | 20.7 |
| LNP52 | MC3 | DC-6-14 | Cholesterol | DMG-mPEG2000 | 39:20:39:2 | 20.7 |
| LNP53 | SM-102 | DC-6-14 | Cholesterol | DMG-mPEG2000 | 39:20:39:2 | 20.7 |
| LNP54 | MD-1 | DC-6-14 | Cholesterol | DMG-mPEG2000 | 39:20:39:2 | 20.5 |
| LNP55 | ALC-0315 | DC-6-14 | Cholesterol | DMG-mPEG2000 | 39:20:39:2 | 20.7 |
| LNP56 | Lipid5 | DC-6-14 | Cholesterol | DMG-mPEG2000 | 39:20:39:2 | 20.6 |
| LNP103 | A9 | DC-6-14 | Cholesterol | DMG-mPEG2000 | 39:20:39:2 | 19.4 |
| LNP104 | Lipid 29 | DC-6-14 | Cholesterol | DMG-mPEG2000 | 39:20:39:2 | 19.9 |
| LNP105 | ATX-100 | DC-6-14 | Cholesterol | DMG-mPEG2000 | 39:20:39:2 | 21.3 |
| LNP106 | Lp01 | DC-6-14 | Cholesterol | DMG-mPEG2000 | 39:20:39:2 | 20.0 |
| LNP107 | TCL053 | DC-6-14 | Cholesterol | DMG-mPEG2000 | 39:20:39:2 | 19.8 |

TABLE 16
Physicochemical Properties

| | Ionizable | Encapsulation | Z-average | | |
|---|---|---|---|---|---|
| | lipid | Efficiency (% EE) | (nm) | PDI | pKa |
| LNP49 | FL-A | 90 | 115.3 | 0.09 | 6.14 |
| LNP50 | FL-B | 89 | 99.5 | 0.06 | 6.51 |
| LNP51 | FL-C | 90 | 126.2 | 0.14 | 6.49 |
| LNP52 | MC3 | 93 | 86.2 | 0.08 | 6.16 |
| LNP53 | SM-102 | 89 | 83.7 | 0.06 | 6.76 |
| LNP54 | MD-1 | 63 | 104.5 | 0.11 | 5.71 |
| LNP55 | ALC-0315 | 84 | 107.1 | 0.10 | 6.49 |
| LNP56 | Lipid5 | 91 | 81.1 | 0.06 | 6.56 |
| LNP103 | A9 | 86 | 101.4 | 0.05 | 6.78 |
| LNP104 | Lipid 29 | 86 | 88.3 | 0.19 | 5.51 |
| LNP105 | ATX-100 | 90 | 128.3 | 0.05 | 5.99 |
| LNP106 | Lp01 | 87 | 146.1 | 0.11 | 6.31 |
| LNP107 | TCL053 | 85 | 104.6 | 0.18 | 6.25 |

### Example 9: Optimization of EDPPC Ratio (0-50 Mol %)

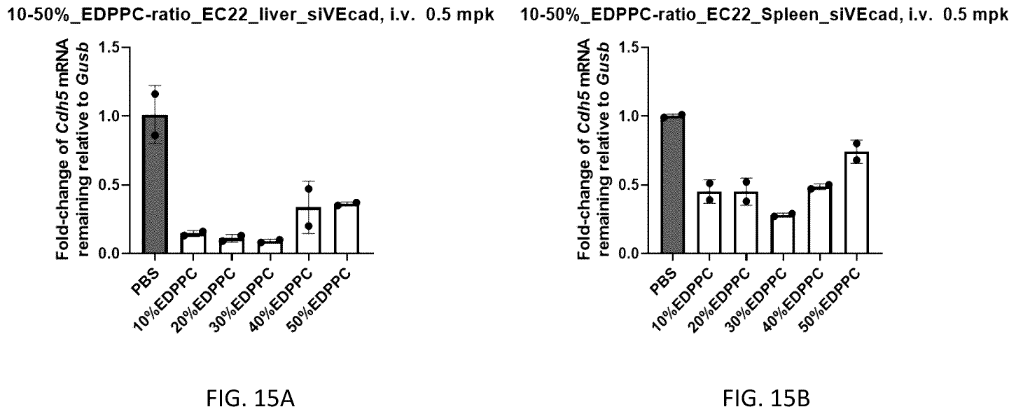

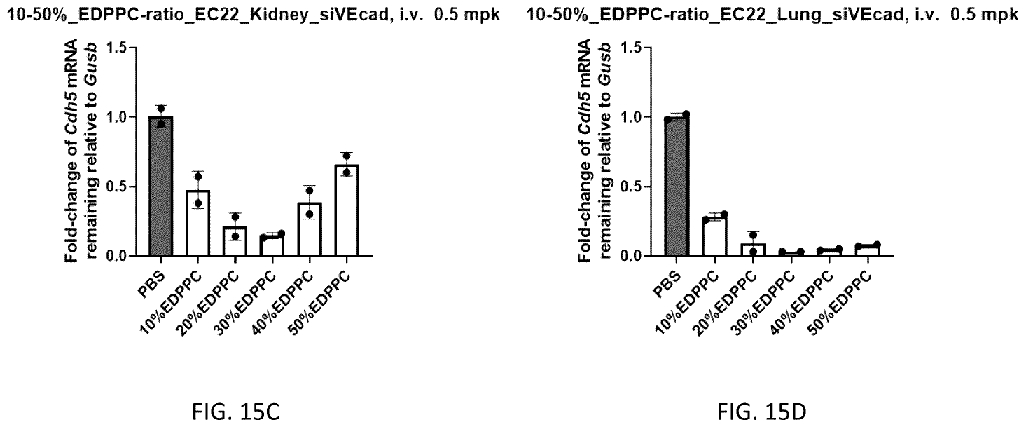

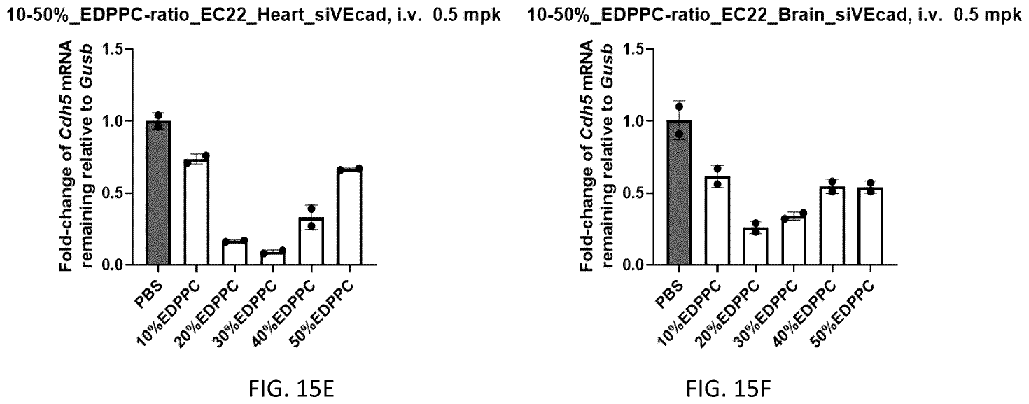

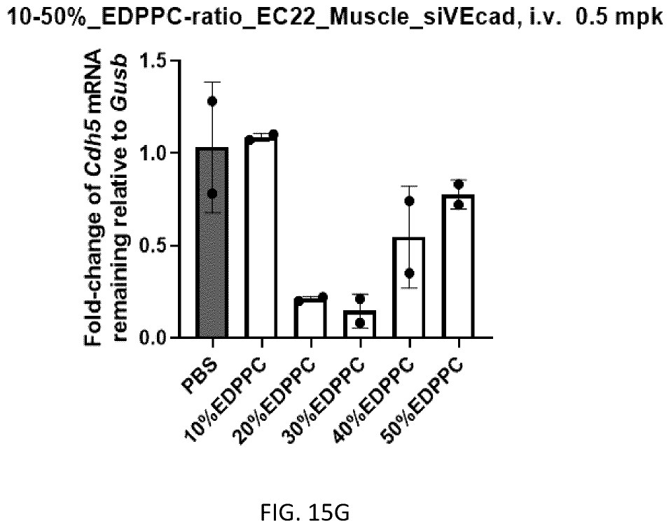

To optimize the cationic lipid ratio for extrahepatic delivery, a series of LNPs were prepared by systematically increasing the percentage of the cationic lipids identified in the screening. The resulting LNPs were tested in vivo with single 0.5 mg/kg siCdh5 administration. LNPs containing 10-50% EDDPC showed efficacious Cdh5 gene silencing in liver, spleen, kidney, lung, and brain, and LNP containing 20-30% EDPPC showed most potent gene silencing in heart and skeletal muscle. The details of the formulations are given in Table 17. The physicochemical properties of the LNPs are given in Table 18. The results are shown in FIG. 15A-15G.

TABLE 17
Formulations

| | Ionizable | Cationic | Zwitterionic | | | | Lipid/RNA |
|---|---|---|---|---|---|---|---|
| | lipid | lipid | phospho-lipid | Sterol | PEG-lipid | Molar ratio | wt ratio |
| LNP57 | FL-A | — | DSPC | Cholesterol | DMG-mPEG2000 | 50:0:10:38.5:1.5 | 18.2 |
| LNP58 | FL-A | EDPPC | — | Cholesterol | DMG-mPEG2000 | 46.75:5:0:46.75:1.5 | 16.9 |
| LNP59 | FL-A | EDPPC | — | Cholesterol | DMG-mPEG2000 | 44.25:10:0:44.25:1.5 | 16.9 |
| LNP60 | FL-A | EDPPC | — | Cholesterol | DMG-mPEG2000 | 39.25:20:0:39.25:1.5 | 16.9 |
| LNP61 | FL-A | EDPPC | — | Cholesterol | DMG-mPEG2000 | 34.25:30:0:34.25:1.5 | 17.0 |
| LNP62 | FL-A | EDPPC | — | Cholesterol | DMG-mPEG2000 | 29.25:40:0:29:25:1.5 | 16.9 |
| LNP63 | FL-A | EDPPC | — | Cholesterol | DMG-mPEG2000 | 24.25:50:0:24.25:1.5 | 16.8 |

TABLE 18
Physicochemical Properties

| | | Encapsulation | Z- | | | Zeta |
|---|---|---|---|---|---|---|
| | | Efficiency | average | | | Potential |
| | EDPPC % | (% EE) | (nm) | PDI | pKa | (mV) |
| LNP57 | 0 | 96 | 93.7 | 0.04 | 6.75 | −5.4 |
| LNP58 | 5 | 92 | 94.0 | 0.06 | 6.44 | −6.6 |
| LNP59 | 10 | 93 | 99.1 | 0.05 | 6.47 | −3.4 |
| LNP60 | 20 | 95 | 90.7 | 0.05 | 6.38 | 3.6 |
| LNP61 | 30 | 97 | 90.1 | 0.06 | 6.53 | 5.9 |
| LNP62 | 40 | 98 | 91.3 | 0.09 | 6.68 | 7.7 |
| LNP63 | 50 | 98 | 94.3 | 0.08 | 6.56 | 12.0 |

### Example 10: Optimization of DPTAP Ratio (0-50 Mol %)

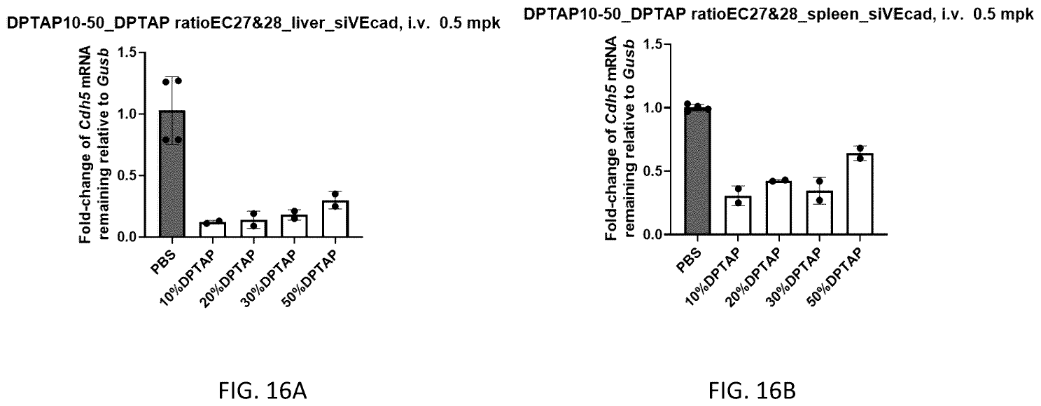

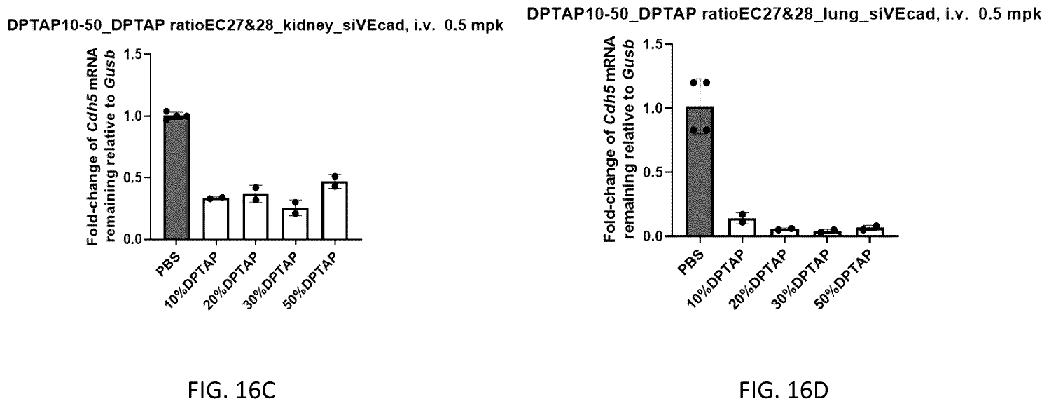

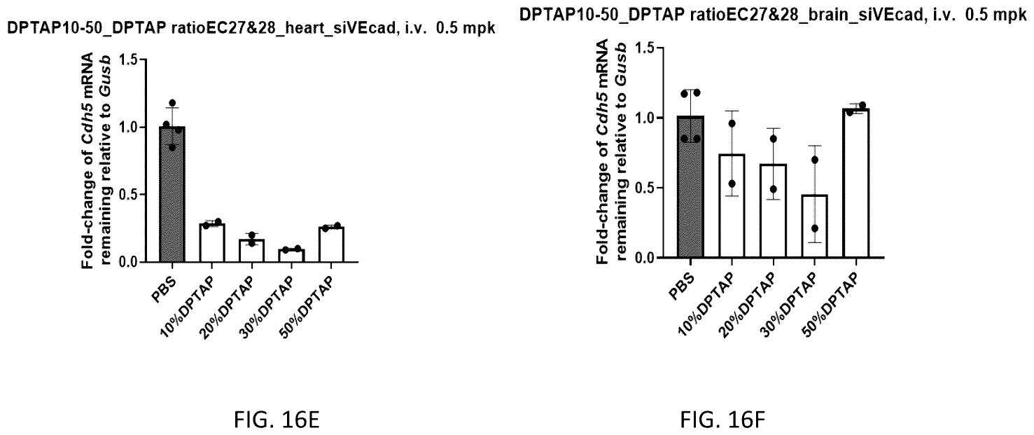

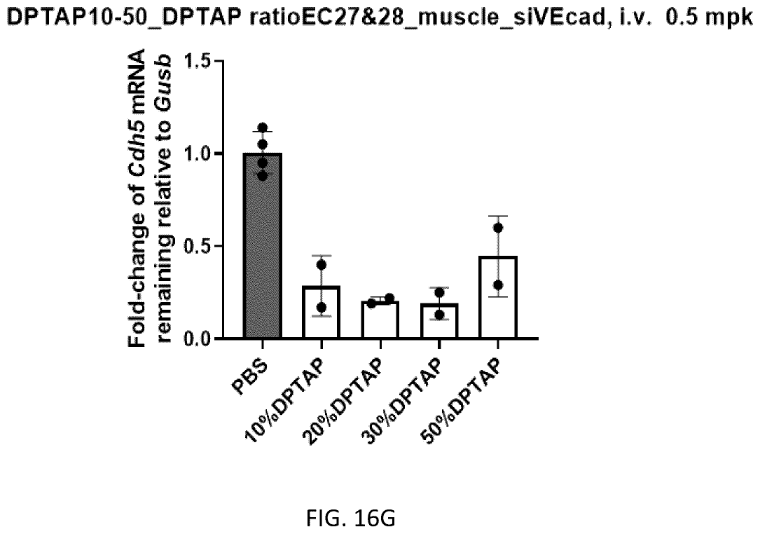

Next, a series of LNPs containing 5-50% DPTAP were prepared and tested in vivo with single 0.5 mg/kg siCdh5 administration. LNPs containing 10-50% DPTAP showed efficacious Cdh5 gene silencing in liver, spleen, kidney, lung, heart, and skeletal muscle. The details of the formulations are given in Table 19. The physicochemical properties of the LNPs are given in Table 20. The results are shown in FIG. 16A-16G.

TABLE 19
Formulations

| | Ionizable | Cationic | | | | Lipid/RNA |
|---|---|---|---|---|---|---|
| | lipid | lipid | Sterol | PEG-lipid | Molar ratio | wt ratio |
| LNP64 | FL-A | DPTAP | Cholesterol | DMG-mPEG2000 | 46.5:5:0:46.5:2 | 20.5 |
| LNP65 | FL-A | DPTAP | Cholesterol | DMG-mPEG2000 | 44:10:0:44:2 | 20.5 |
| LNP66 | FL-A | DPTAP | Cholesterol | DMG-mPEG2000 | 39:20:0:39:2 | 20.6 |
| LNP67 | FL-A | DPTAP | Cholesterol | DMG-mPEG2000 | 34:30:0:34:2 | 20.5 |
| LNP68 | FL-A | DPTAP | Cholesterol | DMG-mPEG2000 | 29:50:0:19:2 | 20.5 |

TABLE 20
Physicochemical Properties

| | | Encapsulation | Z- | | | Zeta |
|---|---|---|---|---|---|---|
| | | Efficiency | average | | | Potential |
| | DPTAP % | (% EE) | (nm) | PDI | pKa | (mV) |
| LNP64 | 5 | 89 | 103.1 | 0.09 | 6.72 | 2.1 |
| LNP65 | 10 | 84 | 88.3 | 0.06 | 6.70 | 1.5 |
| LNP66 | 20 | 89 | 102.4 | 0.10 | 6.27 | 7.6 |
| LNP67 | 30 | 95 | 98.5 | 0.09 | 6.34 | −7.3 |
| LNP68 | 50 | 99 | 133.9 | 0.19 | 6.42 | 4.7 |

### Example 11: Optimization of DC-6-14 Ratio (0-50 Mol %)

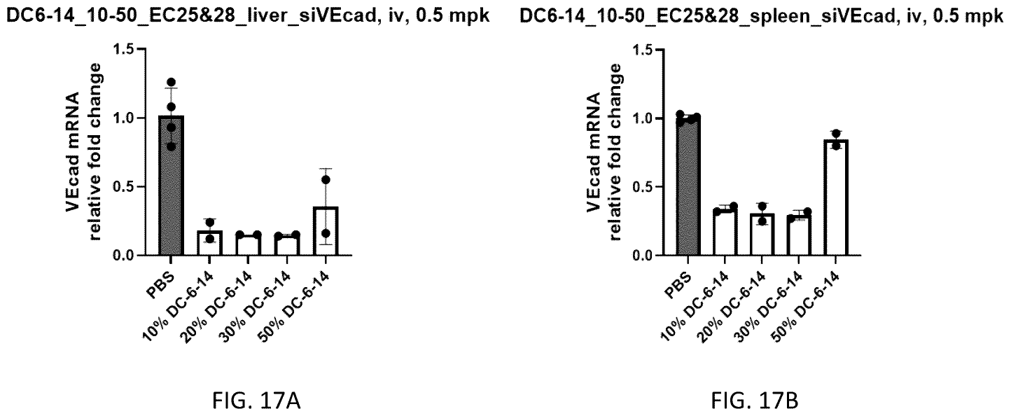

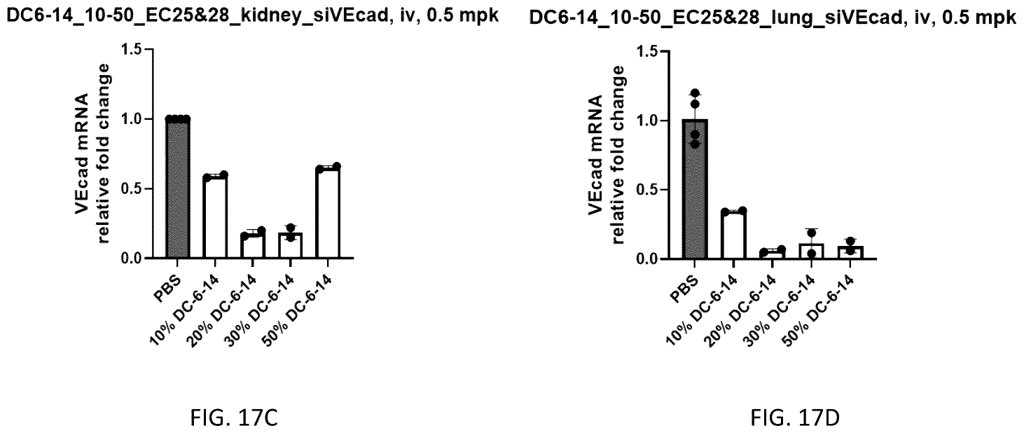

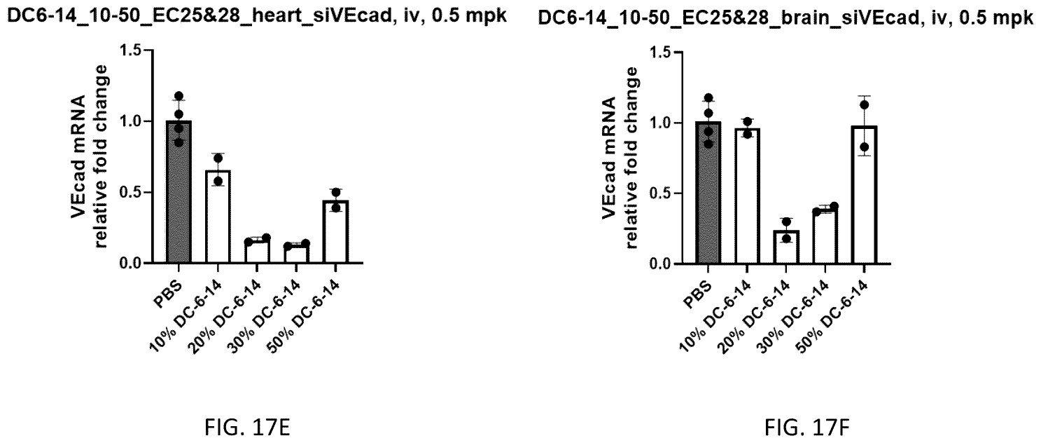

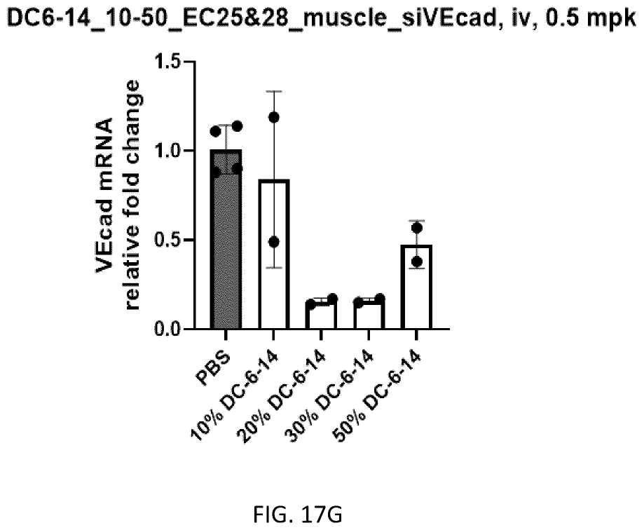

Next, a series of LNPs were prepared containing 5-50% DC-6-14 and tested in vivo with a single 0.5 mg/kg siCdh5 administration. LNPs containing 10-50% DPTAP showed efficacious Cdh5 gene silencing in liver, spleen, kidney, lung, heart, and skeletal muscle. The details of the formulations are given in Table 21. The physicochemical properties of the LNPs are given in Table 22. The results are shown in FIG. 17A-17G.

TABLE 21
Formulations

| | Ionizable | Cationic | | | | Lipid/RNA |
|---|---|---|---|---|---|---|
| | lipid | lipid | Sterol | PEG-lipid | Molar ratio | wt ratio |
| LNP69 | FL-A | DC-6-14 | Cholesterol | DMG-mPEG2000 | 46.5:5:0:46.5:2 | 20.7 |
| LNP70 | FL-A | DC-6-14 | Cholesterol | DMG-mPEG2000 | 46.5:10:0:46.5:2 | 20.7 |
| LNP71 | FL-A | DC-6-14 | Cholesterol | DMG-mPEG2000 | 39:20:0:39:2 | 20.7 |
| LNP72 | FL-A | DC-6-14 | Cholesterol | DMG-mPEG2000 | 34:30:0:34:2 | 20.6 |
| LNP73 | FL-A | DC-6-14 | Cholesterol | DMG-mPEG2000 | 29:50:0:19:2 | 20.8 |

TABLE 22
Physicochemical Properties

| | | Encapsulation | Z- | | | Zeta |
|---|---|---|---|---|---|---|
| | | Efficiency | average | | | Potential |
| | DC-6-14% | (% EE) | (nm) | PDI | pKa | (mV) |
| LNP69 | 5 | 83 | 108.9 | 0.09 | 6.86 | −2.4 |
| LNP70 | 10 | 82 | 92.1 | 0.05 | 6.69 | −1.0 |
| LNP71 | 20 | 90 | 115.3 | 0.09 | 6.14 | 7.0 |
| LNP72 | 30 | 91 | 133.0 | 0.09 | 6.31 | 2.8 |
| LNP73 | 50 | 97 | 112.2 | 0.12 | 6.67 | 5.7 |

### Example 12: 50% DOTAP with Lipids of Formula I or III

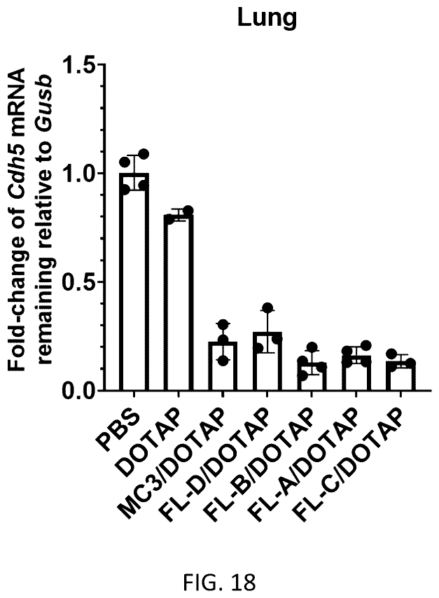

To examine whether 50% DOTAP incorporation into liver-targeting LNPs comprising lipids of formula I or III redirects to the lung, five LNPs with different ionizable lipids were prepared (MC3 as a positive control), along with one LNP without ionizable lipid as a negative control, and their lung endothelial delivery efficiency in vivo was tested using a single 0.4 mg/kg siCdh5 intravenous administration. Three days post administration, the lungs were harvested and Cdh5 mRNA was quantified by RT-qPCR. All the LNPs containing an ionizable lipid selected from FL-A, FL-B, FL-C, and FL-D in addition to 50% DOTAP showed 80-90% Cdh5 gene silencing, indicating that these novel formulations can be used for RNA delivery to the lung. The details of the formulations are given in Table 23. The physicochemical properties of the LNPs are given in Table 24. The results are shown in FIG. 18.

TABLE 23
Formulations

| | Ionizable | Cationic | Zwitterionic | | | | Lipid/RNA |
|---|---|---|---|---|---|---|---|
| | lipid | lipid | phospholipid | Sterol | PEG-lipid | Molar ratio | wt ratio |
| LNP74 | MC3 | DOTAP | DSPC | Cholesterol | DMG-mPEG2000 | 25:50:5:19.25:0.75 | 20.0 |
| LNP75 | FL-D | DOTAP | DSPC | Cholesterol | DMG-mPEG2000 | 25:50:5:19.25:0.75 | 14.9 |
| LNP76 | FL-B | DOTAP | DSPC | Cholesterol | DMG-mPEG2000 | 25:50:5:19.25:0.75 | 15.4 |
| LNP77 | FL-A | DOTAP | DSPC | Cholesterol | DMG-mPEG2000 | 25:50:5:19.25:0.75 | 17.1 |
| LNP78 | FL-C | DOTAP | DSPC | Cholesterol | DMG-mPEG2000 | 25:50:5:19.25:0.75 | 18.5 |
| LNP79 | — | DOTAP | DSPC | Cholesterol | DMG-mPEG2000 | 0:50:10:38.5:1.5 | 17.6 |

TABLE 24
Physicochemical Properties

| | Ionizable | Encapsulation | Z-average | |
|---|---|---|---|---|
| | lipid | Efficiency (% EE) | (nm) | PDI |
| LNP74 | MC3 | 99 | 77.1 | 0.10 |
| LNP75 | FL-D | 99 | 95.5 | 0.07 |
| LNP76 | FL-B | 99 | 74.0 | 0.07 |
| LNP77 | FL-A | 99 | 90.1 | 0.09 |
| LNP78 | FL-C | 99 | 90.3 | 0.06 |
| LNP79 | — | 99 | 86.2 | 0.17 |

### Example 13: 50% DOTAP with Different PEG Lipids

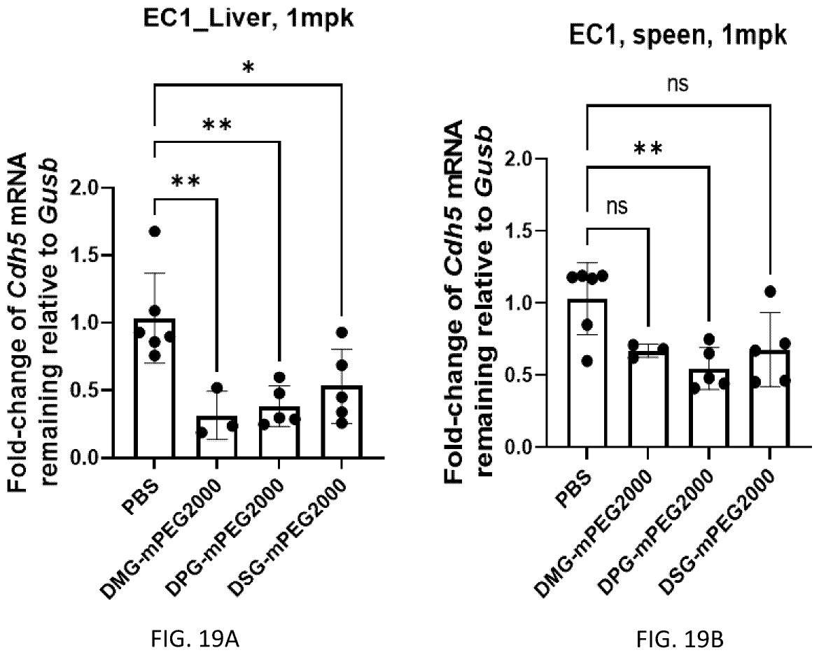

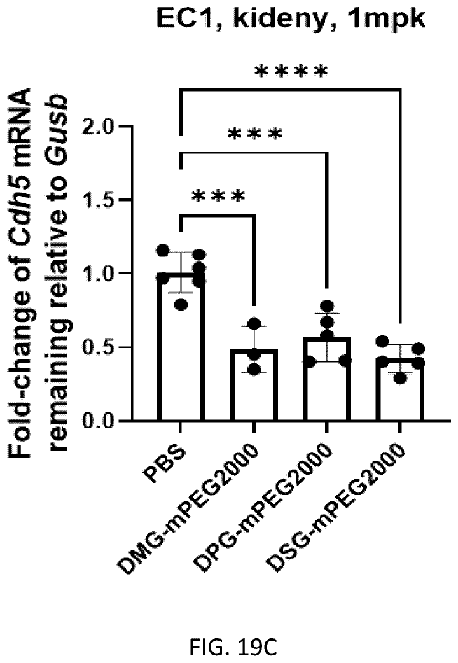

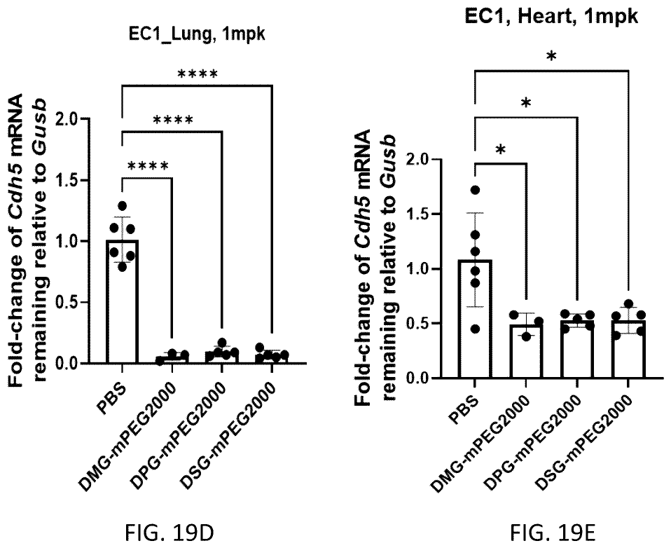

To examine whether alkyl chain length of PEG-lipid affects tissue tropism of LNPs containing 50% DOTAP, three LNPs with different PEG lipids were prepared and tested for their endothelial delivery efficiency in vivo using a single 1.0 mg/kg siCdh5 intravenous administration. Three days post administration, the tissues were harvested and Cdh5 mRNA was quantified by RT-qPCR. All the LNPs showed comparable Cdh5 gene silencing in all the tissues collected. The details of the formulations are given in Table 25. The physicochemical properties of the LNPs are given in Table 26. The results are shown in FIG. 19A-19E.

TABLE 25
Formulations

| | Ionizable | Cationic | Zwitterionic | | | | Lipid/RNA |
|---|---|---|---|---|---|---|---|
| | lipid | lipid | phospholipid | Sterol | PEG-lipid | Molar ratio | wt ratio |
| LNP80 | FL-A | DOTAP | DSPC | Cholesterol | DMG-mPEG2000 | 25:50:5:18.5:1.5 | 17.5 |
| LNP81 | FL-A | DOTAP | DSPC | Cholesterol | DPG-mPEG2000 | 25:50:5:18.5:1.5 | 17.5 |
| LNP82 | FL-A | DOTAP | DSPC | Cholesterol | DSG-mPEG2000 | 25:50:5:18.5:1.5 | 17.5 |

TABLE 26
Physicochemical Properties

| | | Encapsulation | | |
|---|---|---|---|---|
| | | Efficiency | | |
| | PEG-lipid | (% EE) | Z-average (nm) | PDI |
| LNP80 | DMG- | 100 | 81.6 | 0.13 |
| | mPEG2000 | | | |
| LNP81 | DPG- | 100 | 71.9 | 0.15 |
| | mPEG2000 | | | |
| LNP82 | DSG- | 100 | 69.1 | 0.13 |
| | mPEG2000 | | | |

### Example 14: 20% EDOPC with Different Ionizable Lipids

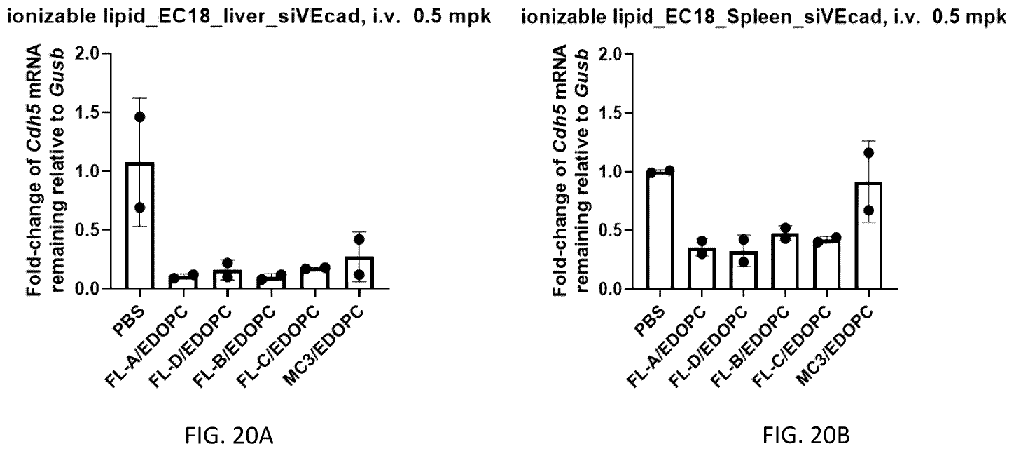

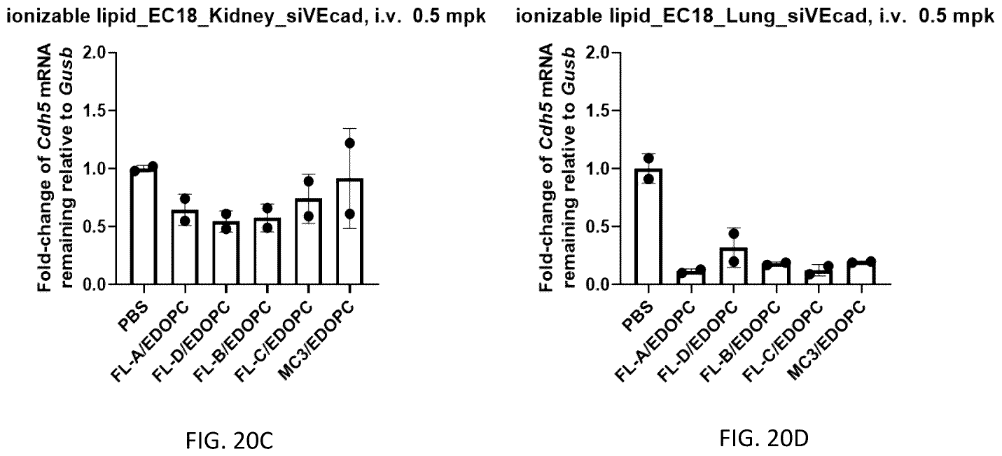

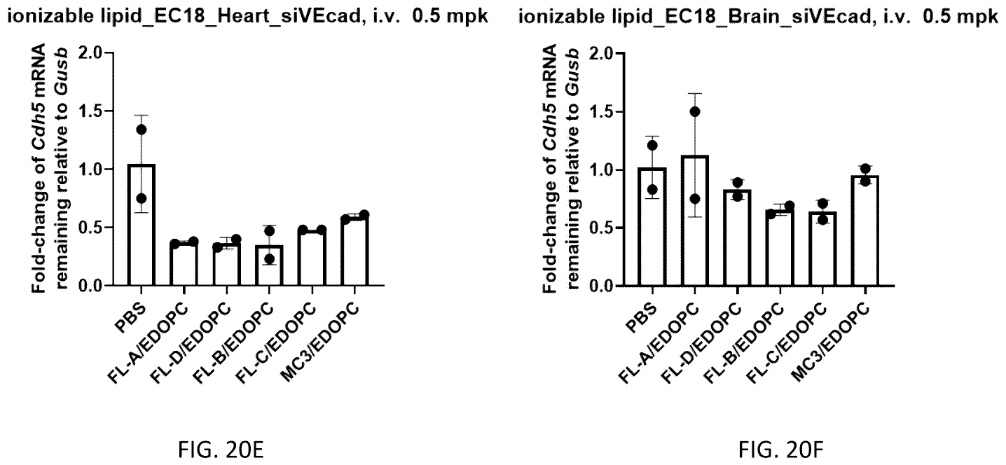

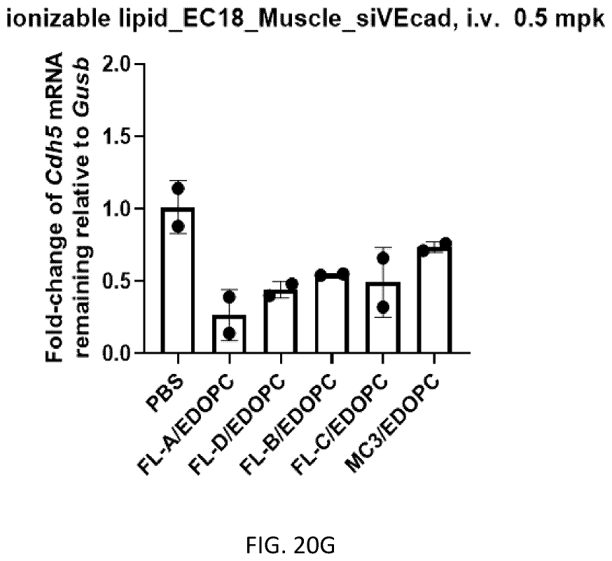

To examine the generalizability of EDOPC for extrahepatic delivery, various ionizable lipids were formulated with 20% EDOPC. LNPs with five different ionizable lipids were prepared and tested for their endothelial delivery efficiency in vivo using a single 0.5 mg/kg siCdh5 intravenous administration. Three days post administration, the tissues were harvested and Cdh5 mRNA was quantified by RT-qPCR. All the LNPs containing 20% EDOPC showed significant Cdh5 gene silencing in all the tissues tested, indicating that these novel formulations can be used for RNA delivery to the endothelial RNA delivery in various tissues. The details of the formulations are given in Table 27. The physicochemical properties of the LNPs are given in Table 28. The results are shown in FIG. 20A-20G.

TABLE 27
Formulations

| | Ionizable | Cationic | | | | Lipid/RNA |
|---|---|---|---|---|---|---|
| | lipid | lipid | Sterol | PEG-lipid | Molar ratio | wt ratio |
| LNP83 | FL-D | EDOPC | Cholesterol | DMG-mPEG2000 | 50:20:28.5:1.5 | 16.5 |
| LNP84 | FL-B | EDOPC | Cholesterol | DMG-mPEG2000 | 50:20:28.5:1.5 | 16.9 |
| LNP85 | FL-A | EDOPC | Cholesterol | DMG-mPEG2000 | 50:20:28.5:1.5 | 18.0 |
| LNP86 | FL-C | EDOPC | Cholesterol | DMG-mPEG2000 | 50:20:28.5:1.5 | 18.9 |
| LNP87 | MC3 | EDOPC | Cholesterol | DMG-mPEG2000 | 50:20:28.5:1.5 | 19.9 |

TABLE 28
Physicochemical Properties

| | Ionizable | Encapsulation | Z-average | | |
|---|---|---|---|---|---|
| | lipid | Efficiency (% EE) | (nm) | PDI | pKa |
| LNP83 | FL-D | 96 | 91.8 | 0.13 | 6.91 |
| LNP84 | FL-B | 97 | 83.8 | 0.10 | 6.85 |
| LNP85 | FL-A | 99 | 78.5 | 0.09 | 6.83 |
| LNP86 | FL-C | 97 | 141.1 | 0.21 | 7.01 |
| LNP87 | MC3 | 98 | 68.8 | 0.12 | 6.73 |

### Example 15:18.5% EDOPC with Different PEG Lipids

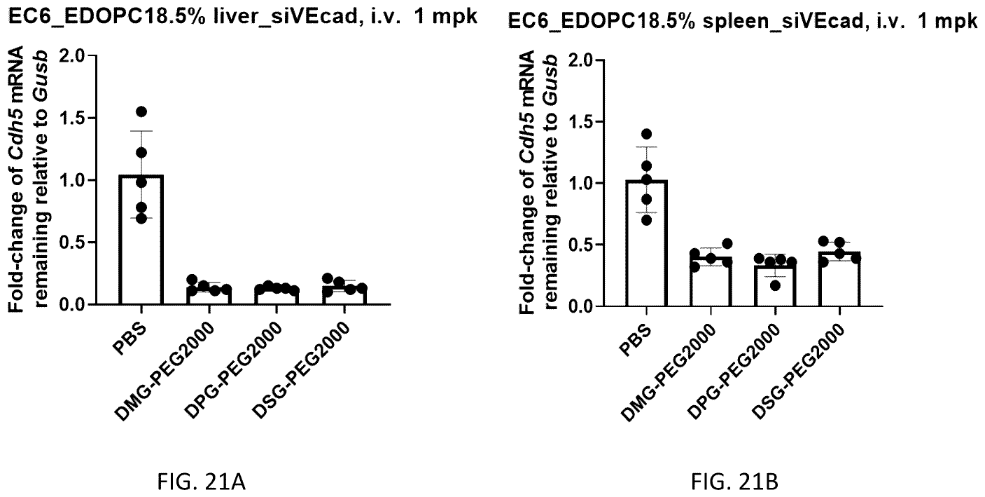

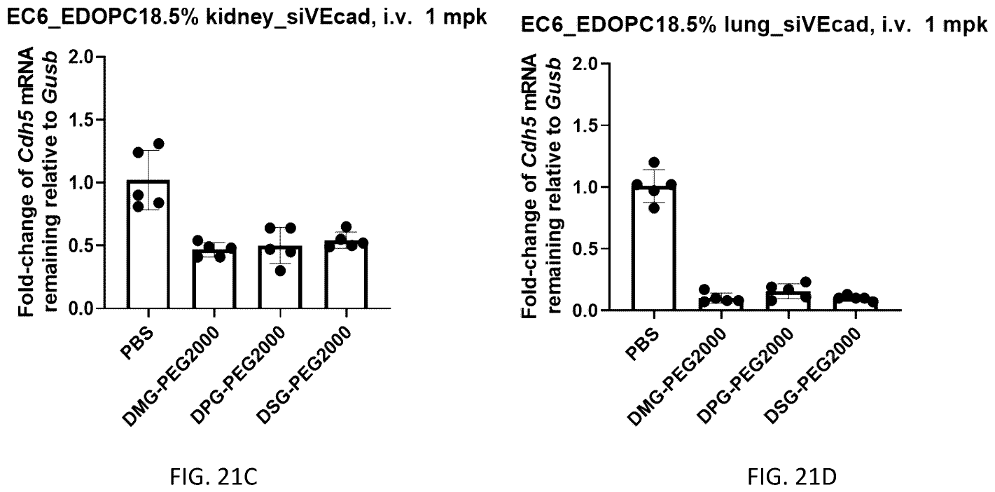

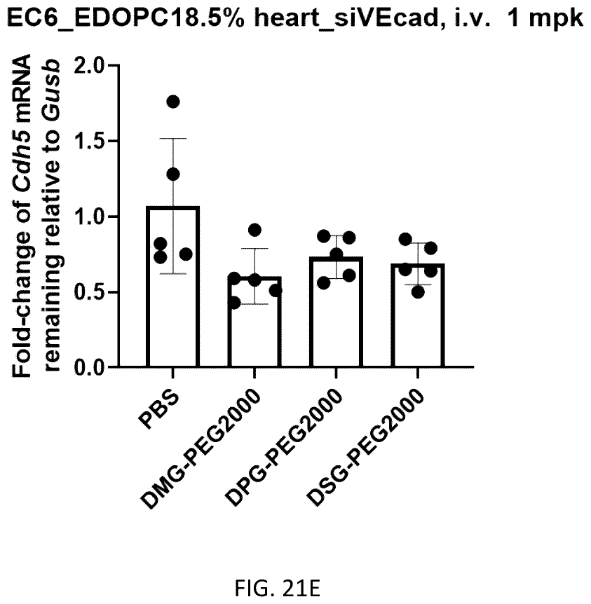

To examine whether alkyl chain length of PEG-lipid affects tissue tropism of LNPs containing 18.5% EDOPC, three LNPs with different PEG lipids were prepared and tested for their endothelial delivery efficiency in vivo using a single 1.0 mg/kg siCdh5 intravenous administration. Three days post administration, the tissues were harvested and Cdh5 mRNA was quantified by RT-qPCR. All the LNPs showed comparable Cdh5 gene silencing in all the tissues collected. The details of the formulations are given in Table 29. The physicochemical properties of the LNPs are given in Table 30. The results are shown in FIG. 21A-21E.

TABLE 29
Formulations

| | Ionizable | Cationic | | | | Lipid/RNA |
|---|---|---|---|---|---|---|
| | lipid | lipid | Sterol | PEG-lipid | Molar ratio | wt ratio |
| LNP88 | FL-A | EDOPC | Cholesterol | DMG-mPEG2000 | 50:18.5:30:1.5 | 17.8 |
| LNP89 | FL-A | EDOPC | Cholesterol | DPG-mPEG2000 | 50:18.5:30:1.5 | 17.8 |
| LNP90 | FL-A | EDOPC | Cholesterol | DSG-mPEG2000 | 50:18.5:30:1.5 | 17.8 |

TABLE 30
Physicochemical Properties

| | Encapsulation | Z-average | |
|---|---|---|---|
| | Efficiency (% EE) | (nm) | PDI |
| LNP88 | 98 | 102.8 | 0.05 |
| LNP89 | 99 | 95.2 | 0.06 |
| LNP90 | 99 | 102.3 | 0.08 |

### Example 16: 10% DPTAP with Different Ionizable Lipids

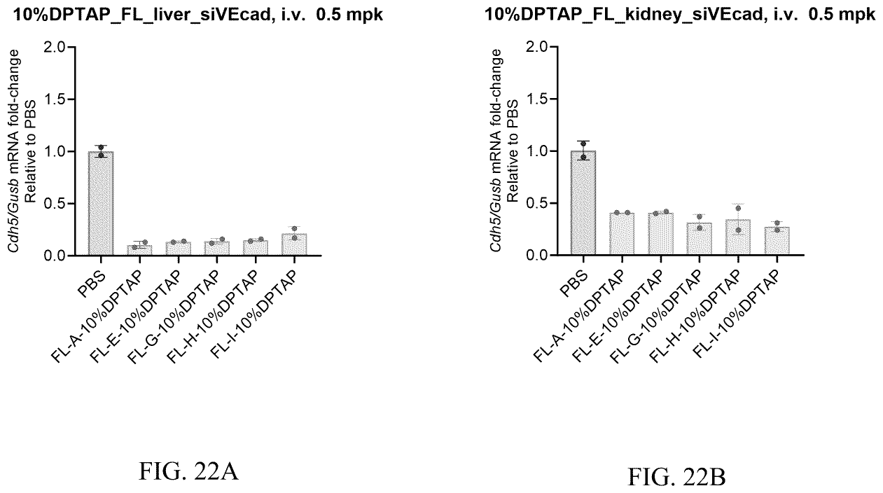

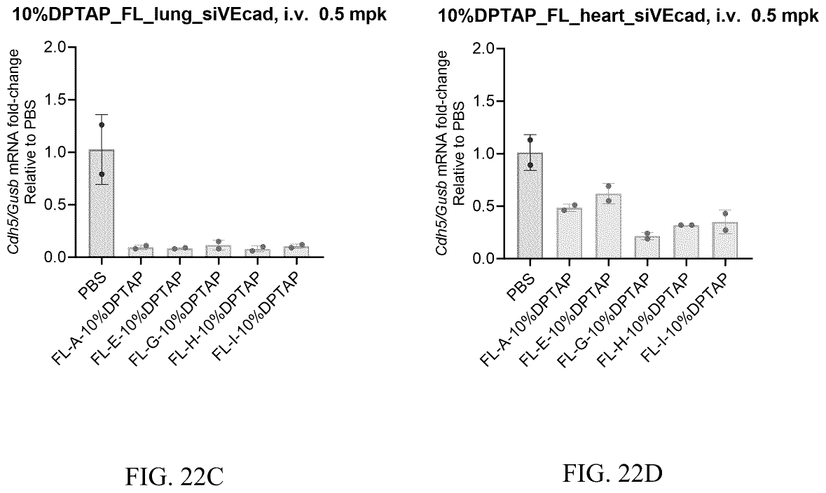

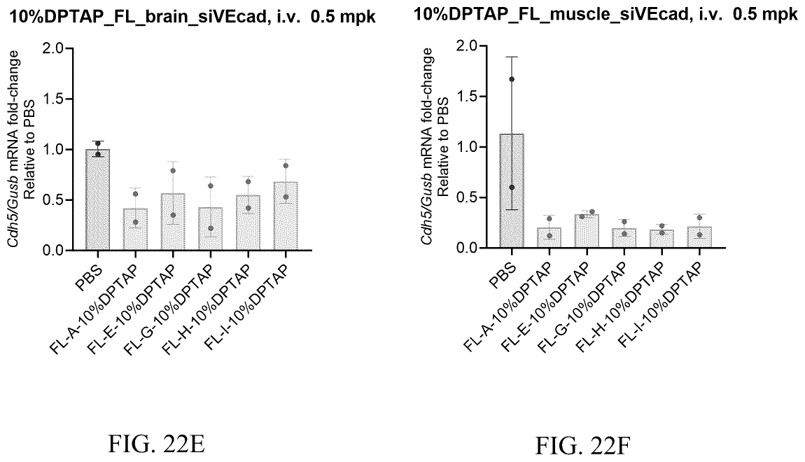

To examine the generalizability of DPTAP for extrahepatic delivery, various ionizable lipids were formulated with 10% DPTAP. LNPs with six different ionizable lipids were prepared and tested for their endothelial delivery efficiency in vivo using a single 0.5 mg/kg siCdh5 intravenous administration. Three days post administration, the tissues were harvested and Cdh5 mRNA was quantified by RT-qPCR. All the LNPs containing 10% DPTAP showed significant Cdh5 gene silencing in all the tissues tested, indicating that these novel formulations can be used for RNA delivery to the endothelial RNA delivery in various tissues. The details of the formulations are given in Table 31. The physicochemical properties of the LNPs are given in Table 32. The results are shown in FIG. 22A-22F.

TABLE 31
Formulations

| | Ionizable | Cationic | | | | Lipid/RNA |
|---|---|---|---|---|---|---|
| | lipid | lipid | Sterol | PEG-lipid | Molar ratio | wt ratio |
| LNP91 | FL-A | DPTAP | Cholesterol | DMG-mPEG2000 | 50:10:38:2 | 16.6 |
| LNP92 | FL-E | DPTAP | Cholesterol | DMG-mPEG2000 | 50:10:38:2 | 15.1 |
| LNP93 | FL-F | DPTAP | Cholesterol | DMG-mPEG2000 | 50:10:38:2 | 15.6 |
| LNP94 | FL-G | DPTAP | Cholesterol | DMG-mPEG2000 | 50:10:38:2 | 15.7 |
| LNP95 | FL-H | DPTAP | Cholesterol | DMG-mPEG2000 | 50:10:38:2 | 15.2 |
| LNP96 | FL-I | DPTAP | Cholesterol | DMG-mPEG2000 | 50:10:38:2 | 16.6 |

TABLE 32
Physicochemical Properties

| | Ionizable | Encapsulation | | |
|---|---|---|---|---|
| | lipid | Efficiency (% EE) | Z-average (nm) | PDI |
| LNP91 | FL-A | 95 | 114.0 | 0.06 |
| LNP92 | FL-E | 95 | 102.1 | 0.12 |
| LNP93 | FL-F | 93 | 117.9 | 0.09 |
| LNP94 | FL-G | 94 | 124.4 | 0.07 |
| LNP95 | FL-H | 92 | 90.8 | 0.09 |
| LNP96 | FL-I | 92 | 110.6 | 0.10 |

1. A composition comprising a plurality of lipid nanoparticles, wherein the lipid nanoparticles comprise an agent, an ionizable lipid and a fully saturated cationic lipid, wherein said lipid nanoparticle does not contain a zwitterionic phospholipid.

2. The composition of claim 1, wherein the lipid nanoparticles have an apparent ionization constant (pKa) between about 4.5 and about 7.

3.-5. (canceled)

6. The composition of any one of claim 1, wherein the cationic lipid is less than about 50 mol percent of the lipid nanoparticle.

7.-9. (canceled)

10. The composition of claim 1, further comprising a sterol.

11. The composition of claim 1, further comprising a PEG-lipid.

12.-20. (canceled)

21. The composition of claim 1, wherein the ionizable lipid is a lipid according to formula I or II or a pharmaceutically acceptable salt thereof: 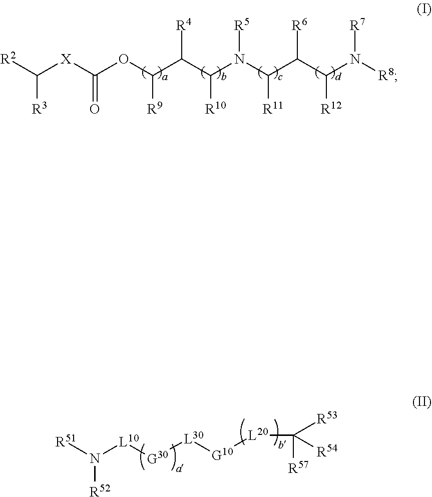
wherein
X is —NR¹— or —O—;
R¹ is a hydrogen atom, a hydrocarbon group having 6 to 24 atoms, or R²¹-L¹-R²²—;
R²¹ is a hydrocarbon group having 1 to 24 carbon atoms; 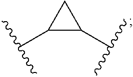 [SMILES: *C1CC1*]
L¹ is —O(CO)O—, —O(CO)—, —(CO)O—, —O—, or
R²² is a divalent hydrocarbon linking group having 1 to 18 carbon atoms;
R² and R³ are each independently a hydrogen atom, a hydrocarbon group having 3 to 24 carbon atoms, or R³¹-L¹-R³²—;
R³¹ is a hydrocarbon group having 1 to 24 carbon atoms;
L² is —O(CO)O—, —O(CO)—, —(CO)O—, —O—, or 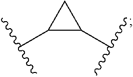 [SMILES: *C1CC1*]
R³² is a divalent hydrocarbon linking group having 1 to 18 carbon atoms;
R⁴, R⁵, R⁶, R⁷, R⁸, R⁹, R¹⁰, R¹¹, and R¹² are each independently a hydrogen atom or an alkyl group having 1 to 18 carbon atoms optionally substituted with R³³, where one or more pairs of groups selected from R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸ may be linked to each other to form a 4- to 7-membered ring of carbon and nitrogen atoms which may contain an oxygen atom;
R³³ is a hydroxyl group, a carboxyl group, —NR⁴⁵R⁴⁶, —O(CO)O—R⁴¹, —O(CO)—R⁴², —(CO)O—R⁴³, —O—R⁴⁴, or an aryl or heteroaryl group optionally substituted with R³⁴;
R³⁴ is an alkyl group having 1 to 18 carbon atoms, a hydroxyl group, a carboxyl group, —NR⁴⁵R⁴⁶, —O(CO)O—R⁴¹, —O(CO)—R⁴², —(CO)O—R⁴³, or —O—R⁴⁴;
R⁴¹, R⁴², R⁴³, R⁴⁴, R⁴⁵, and R⁴⁶ are each independently a hydrocarbon group having 1 to 18 carbon atoms;
a, b, c, and d are each independently an integer from 0 to 3, where a+b is 1 or more and c+d is 1 or more;
R⁵¹ and R⁵² are each independently an alkyl group having 1 to 18 carbon atoms optionally substituted with R³⁵;
R³⁵ is a hydroxyl group, -G²⁰-CH(R⁵⁵)(R⁵⁶), —N(R⁵⁸)(R⁵⁹), or -G²⁰-R⁶⁰;
G²⁰ is —(CO)O— or —O(CO)—;
R⁵⁵ and R⁵⁶ are each independently hydrogen or an alkyl group having 1 to 18 carbon atoms;
R⁵⁸ and R⁵⁹ are each independently hydrogen or a cycloalkyl group having 3 to 6 carbon atoms, optionally substituted with R³⁶;
R⁶⁰ is alkyl group having 1 to 18 carbon atoms or a cycloalkyl group having 3 to 6 carbon atoms;
R³⁶ is —N(R⁶¹)(R⁶²), or -G²⁰-R⁶⁵;
R⁶¹ and R⁶² are each independently hydrogen or a cycloalkyl group having 3 to 6 carbon atoms;
R⁶⁵ is an alkyl group having 1 to 18 carbon atoms, -L⁴⁰-CH(R⁶⁶)(R⁶⁷), or -G²⁰-R⁶⁶,
L⁴⁰ is a divalent alkyl group containing 1 to 6 carbon atoms;
R⁶⁶ and R⁶⁷ are each independently an alkyl group containing 1 to 10 carbon atoms or an alkoxy group containing 1 to 10 carbon atoms;
L¹⁰ is a divalent alkyl group containing 1 to 10 carbon atoms;
G³⁰ is —S(CO)N(R⁶⁴)—;
R⁶⁴ is -L³⁰-G²⁰-CH(R⁵⁵)(R⁵⁶);
a′ is 0 or 1;
G¹⁰ is G²⁰, —O(CO)O—, or —N(R⁶³) C(O)—;
R⁶³ is an alkyl group containing 1 to 18 carbon atoms;
L²⁰ is a divalent alkyl group containing 1 to 6 carbon atoms;
b′ is 0 or 1;
R⁵³, R⁵⁴, and R⁵⁷ are each independently hydrogen or an alkyl group containing 1 to 18 carbon atoms optionally substituted with R³⁶; and
L³⁰ is a single bond or an alkyl group containing 1 to 18 carbon atoms.

22. The composition of claim 1, wherein the ionizable lipid is a lipid according to formula VII or a pharmaceutically acceptable salt thereof: 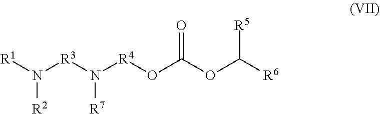
wherein
R¹ and R² are each independently a hydrocarbon group having 1 to 18 carbon atoms, and R³ is a hydrocarbon group having 2 to 8 carbon atoms, wherein the hydrocarbon groups represented by R¹, R², and R³ are each independently optionally substituted with one or more substituents selected from —OH, COOH, —NR⁵¹R⁵², —OC(O)O—R⁵³, —C(O)O—R⁵⁴, —OC(O)—R⁵⁵, and —O—R⁵⁶;
R⁴ is a hydrocarbon group having 1 to 8 carbon atoms;
R⁵ and R⁶ are each independently a hydrocarbon group having 1 to 8 carbon atoms or —R⁸-L¹-R⁹, excluding a case where both R⁵ and R⁶ are hydrocarbon groups having 1 to 8 carbon atoms;
R⁷ is —R¹⁰-L²-R¹¹-L³-R¹²;
R⁵¹ and R⁵² are each independently a hydrocarbon group having 1 to 8 carbon atoms;
R⁵³, R⁵⁴, R⁵⁵, and R⁵⁶ are each independently a hydrocarbon group having 1 to 24 carbon atoms;
the hydrocarbon groups represented by R⁵³, R⁵⁴, R⁵⁵, and R⁵⁶ are each independently optionally substituted with an aryl group having 6 to 20 carbon atoms or —S—R⁵⁸, wherein the aryl group having 6 to 20 carbon atoms is optionally substituted with —OH, COOH, —NR⁵¹R⁵², —OC(O)O—R⁵³, —C(O)O—R⁵⁴, —OC(O)—R⁵⁵, —O—R⁵⁶, or —R⁵⁹—R⁵⁷, wherein
R⁵⁸ and R⁵⁹ are each independently a hydrocarbon group having 1 to 12 carbon atoms;
R⁵⁷ is —OH, COOH, —NR⁶¹R⁶², —OC(O)O—R⁶³, —C(O)O—R⁶⁴, —OC(O)—R⁶⁵, or —O—R⁶⁶;
R⁶¹ and R⁶² are each independently a hydrocarbon group having 1 to 8 carbon atoms;
R⁶³, R⁶⁴, R⁶⁵, and R⁶⁶ are each independently a hydrocarbon group having 1 to 24 carbon atoms;
the hydrocarbon groups represented by R⁶³, R⁶⁴, R⁶⁵, and R⁶⁶ are each independently optionally substituted with an aryl group having 6 to 20 carbon atoms or —S—R⁶⁸ wherein the aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or —R⁶⁹—R⁶⁷,
R⁶⁸ and R⁶⁹ are each independently a hydrocarbon group having 1 to 12 carbon atoms;
R⁶⁷ is —OH, COOH, —NR⁶¹R⁶², —OC(O)O—R⁶³, —C(O)O—R⁶⁴, —OC(O)—R⁶⁵, or —O—R⁶⁶;
L¹, L², and L³ are each independently —OC(O)O—, —C(O)O—, —OC(O)—, or —O—;
R⁸ is a hydrocarbon group having 1 to 12 carbon atoms;
R⁹ is a hydrocarbon group having 1 to 24 carbon atoms;
R¹⁰ is a hydrocarbon group having 1 to 8 carbon atoms;
R¹¹ is a hydrocarbon group having 1 to 24 carbon atoms;
R¹² is a hydrocarbon group having 1 to 24 carbon atoms;
the hydrocarbon groups represented by R⁹ and R¹² are each independently optionally substituted with an aryl group, —OC(O)O—R⁵³, —C(O)O—R⁵⁴, —OC(O)—R⁵⁵, or —S—R⁵⁸; and
the hydrocarbon groups represented by R¹¹ are each independently optionally substituted with —OC(O)O—R⁵³, —C(O)O—R⁵⁴, or —OC(O)—R⁵⁵.

23. The composition of claim 1, wherein the ionizable lipid is a lipid according to formula VIII or a pharmaceutically acceptable salt thereof: 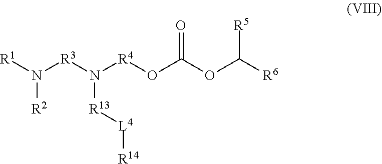
wherein
R¹ and R² are each independently a hydrocarbon group having 1 to 18 carbon atoms, and R³ is a hydrocarbon group having 2 to 8 carbon atoms, wherein the hydrocarbon groups represented by R¹, R², and R³ are each independently optionally substituted with —OH, COOH, —NR⁵¹R⁵², —OC(O)O—R⁵³, —C(O)O—R⁵⁴, —OC(O)—R⁵⁵, or —O—R⁵⁶;
R⁴ is a hydrocarbon group having 1 to 8 carbon atoms;
R⁵ and R⁶ are each independently a hydrocarbon group having 1 to 8 carbon atoms or —R⁸-L¹-R⁹, excluding a case where both R⁵ and R⁶ are hydrocarbon groups having 1 to 8 carbon atoms;
L¹ and L⁴ are each independently —OC(O)O—, —C(O)O—, —OC(O)—, or —O—;
R⁸ is a hydrocarbon group having 1 to 12 carbon atoms;
R⁹ is a hydrocarbon group having 1 to 24 carbon atoms, wherein the hydrocarbon group represented by R⁹ is optionally substituted with an aryl group, —OC(O)O—R⁵³, —C(O)O—R⁵⁴, —OC(O)—R⁵⁵, or —S—R⁵⁸;
R⁵¹ and R⁵² are each independently a hydrocarbon group having 1 to 8 carbon atoms;
R⁵³, R⁵⁴, R⁵⁵, and R⁵⁶ are each independently a hydrocarbon group having 1 to 24 carbon atoms;
the hydrocarbon groups represented by R⁵³, R⁵⁴, R⁵⁵, and R⁵⁶ are each independently optionally substituted with an aryl group having 6 to 20 carbon atoms or —S—R⁵⁸, wherein the aryl group having 6 to 20 carbon atoms is optionally substituted with —OH, COOH, —NR⁵¹R⁵², —OC(O)O—R⁵³, —C(O)O—R⁵⁴, —OC(O)—R⁵⁵, —O—R⁵⁶, or —R⁵⁹—R⁵⁷;
R⁵⁸ and R⁵⁹ are each independently a hydrocarbon group having 1 to 12 carbon atoms;
R⁵⁷ is —OH, COOH, —NR⁵¹R⁵², —OC(O)O—R⁵³, —C(O)O—R⁵⁴, —OC(O)—R⁵⁵, or —O—R⁵⁶;
R¹³ is a hydrocarbon group having 1 to 8 carbon atoms;
R¹⁴ is —R¹⁵-L⁵-R¹⁶, wherein R¹⁵ is a hydrocarbon group having 1 to 24 carbon atoms, L⁵ is —OC(O)O—, —C(O)O—, —OC(O)—, or —O—, and R¹⁶ is a hydrocarbon group having 1 to 24 carbon atoms;
the hydrocarbon group having 1 to 24 carbon atoms represented by R¹⁵ is optionally substituted with —OC(O)O—R⁵³, —C(O)O—R⁵⁴, or —OC(O)—R⁵⁵; and
the hydrocarbon group having 1 to 24 carbon atoms represented by R¹⁶ is optionally substituted with an aryl group having 6 to 20 carbon atoms, —OC(O)O—R⁵³, —C(O)O—R⁵⁴, —OC(O)—R⁵⁵ or —S—R⁵⁸.

24.-28. (canceled)

29. The composition of claim 1, wherein the cationic lipid is a lipid according to formula IV: 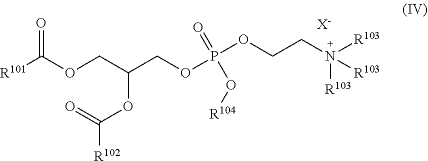
wherein:
R¹⁰¹ and R¹⁰² are each independently optionally substituted (C₈-C₂₄) alkyl or optionally substituted (C₈-C₂₄) alkenyl;
R¹⁰³ is independently in each instance optionally substituted (C₁-C₆) alkyl;
R¹⁰⁴ is optionally substituted (C₁-C₆) alkyl; and
X⁻ is a monovalent anion.

30-31. (canceled)

32. The composition of claim 1, wherein the cationic lipid is a lipid according to formula V: 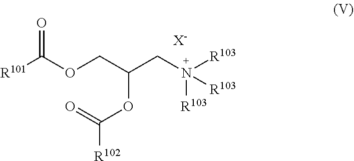
wherein:
R¹⁰¹ and R¹⁰² are each independently optionally substituted (C₈-C₂₄) alkyl or optionally substituted (C₈-C₂₄) alkenyl;
R¹⁰³ is independently in each instance optionally substituted (C₁-C₆) alkyl; and
X⁻ is a monovalent anion.

33-34. (canceled)

35. The composition of claim 1, wherein the cationic lipid is a lipid according to formula VI: 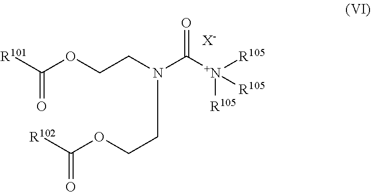
wherein:
R¹⁰¹ and R¹⁰² are each independently optionally substituted (C₈-C₂₄) alkyl or optionally substituted (C₈-C₂₄) alkenyl;
R¹⁰⁵ are each independently (C₁-C₆) alkyl; and
X⁻ is a monovalent anion.

36-38. (canceled)

39. A composition comprising a plurality of lipid nanoparticles, wherein the lipid nanoparticles comprise an agent, an ionizable lipid, and a cationic lipid, wherein the ionizable lipid is a lipid according to formula I or a pharmaceutically acceptable salt thereof: 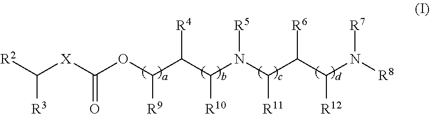
wherein
X is —NR¹— or —O—;
R¹ is a hydrogen atom, a hydrocarbon group having 6 to 24 atoms, or R²¹-L¹-R²²—;
R²¹ is a hydrocarbon group having 1 to 24 carbon atoms;
L¹ is —O(CO)O—, —O(CO)—, —(CO)O—, —O—, or 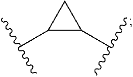 [SMILES: *C1CC1*]
R²² is a divalent hydrocarbon linking group having 1 to 18 carbon atoms;
R² and R³ are each independently a hydrogen atom, a hydrocarbon group having 3 to 24 carbon atoms, or R³¹-L¹-R³²—;
R³¹ is a hydrocarbon group having 1 to 24 carbon atoms;
L² is —O(CO)O—, —O(CO)—, —(CO)O—, —O—, or 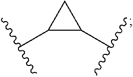 [SMILES: *C1CC1*]
R³² is a divalent hydrocarbon linking group having 1 to 18 carbon atoms;
R⁴, R⁵, R⁶, R⁷, R⁸, R⁹, R¹⁰, R¹¹, and R¹² are each independently a hydrogen atom or an alkyl group having 1 to 18 carbon atoms optionally substituted with R³³, where one or more pairs of groups selected from R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸ may be linked to each other to form a 4- to 7-membered ring of carbon and nitrogen atoms which may contain an oxygen atom;
R³³ is a hydroxyl group, a carboxyl group, —NR⁴⁵R⁴⁶, —O(CO)O—R⁴¹, —O(CO)—R⁴², —(CO)O—R⁴³, —O—R⁴⁴, or an aryl or heteroaryl group optionally substituted with R³⁴,
R³⁴ is an alkyl group having 1 to 18 carbon atoms, a hydroxyl group, a carboxyl group, —NR⁴⁵R⁴⁶, —O(CO)O—R⁴¹, —O(CO)—R⁴², —(CO)O—R⁴³, or —O—R⁴⁴;
R⁴¹, R⁴², R⁴³, R⁴⁴, R⁴⁵, and R⁴⁶ are each independently a hydrocarbon group having 1 to 18 carbon atoms; and
a, b, c, and d are each independently an integer from 0 to 3, where a+b is 1 or more and c+d is 1 or more.

40-41. (canceled)

42. A composition comprising a plurality of lipid nanoparticles, wherein the lipid nanoparticles comprise an agent, an ionizable lipid, and a cationic lipid, wherein the ionizable lipid is a lipid according to formula VII or a pharmaceutically acceptable salt thereof: 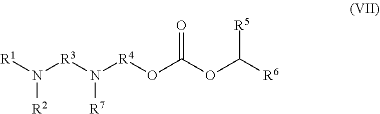
wherein
R¹ and R² are each independently a hydrocarbon group having 1 to 18 carbon atoms, and R³ is a hydrocarbon group having 2 to 8 carbon atoms, wherein the hydrocarbon groups represented by R¹, R², and R³ are each independently optionally substituted with one or more substituents selected from —OH, COOH, —NR⁵¹R⁵², —OC(O)O—R⁵³, —C(O)O—R⁵⁴, —OC(O)—R⁵⁵, and —O—R⁵⁶;
R⁴ is a hydrocarbon group having 1 to 8 carbon atoms;
R⁵ and R⁶ are each independently a hydrocarbon group having 1 to 8 carbon atoms or —R⁸-L¹-R⁹, excluding a case where both R⁵ and R⁶ are hydrocarbon groups having 1 to 8 carbon atoms;
R⁷ is —R¹⁰-L²-R¹¹-L³-R¹²;
R⁵¹ and R⁵² are each independently a hydrocarbon group having 1 to 8 carbon atoms;
R⁵³, R⁵⁴, R⁵⁵, and R⁵⁶ are each independently a hydrocarbon group having 1 to 24 carbon atoms;
the hydrocarbon groups represented by R⁵³, R⁵⁴, R⁵⁵, and R⁵⁶ are each independently optionally substituted with an aryl group having 6 to 20 carbon atoms or —S—R⁵⁸, wherein the aryl group having 6 to 20 carbon atoms is optionally substituted with —OH, COOH, —NR⁵¹R⁵², —OC(O)O—R⁵³, —C(O)O—R⁵⁴, —OC(O)—R⁵⁵, —O—R⁵⁶, or —R⁵⁹—R⁵⁷ wherein
R⁵⁸ and R⁵⁹ are each independently a hydrocarbon group having 1 to 12 carbon atoms;
R⁵⁷ is —OH, COOH, —NR⁶¹R⁶², —OC(O)O—R⁶³, —C(O)O—R⁶⁴, —OC(O)—R⁶⁵, or —O—R⁶⁶;
R⁶¹ and R⁶² are each independently a hydrocarbon group having 1 to 8 carbon atoms;
R⁶³, R⁶⁴, R⁶⁵, and R⁶⁶ are each independently a hydrocarbon group having 1 to 24 carbon atoms;
the hydrocarbon groups represented by R⁶³, R⁶⁴, R⁶⁵, and R⁶⁶ are each independently optionally substituted with an aryl group having 6 to 20 carbon atoms or —S—R⁶⁸ wherein the aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or —R⁶⁹—R⁶⁷,
R⁶⁸ and R⁶⁹ are each independently a hydrocarbon group having 1 to 12 carbon atoms;
R⁶⁷ is —OH, COOH, —NR⁶¹R⁶², —OC(O)O—R⁶³, —C(O)O—R⁶⁴, —OC(O)—R⁶⁵, or —O—R⁶⁶;
L¹, L², and L³ are each independently —OC(O)O—, —C(O)O—, —OC(O)—, or —O—;
R⁸ is a hydrocarbon group having 1 to 12 carbon atoms;
R⁹ is a hydrocarbon group having 1 to 24 carbon atoms;
R¹⁰ is a hydrocarbon group having 1 to 8 carbon atoms;
R¹¹ is a hydrocarbon group having 1 to 24 carbon atoms;
R¹² is a hydrocarbon group having 1 to 24 carbon atoms;
the hydrocarbon groups represented by R⁹ and R¹² are each independently optionally substituted with an aryl group, —OC(O)O—R⁵³, —C(O)O—R⁵⁴, —OC(O)—R⁵⁵, or —S—R⁵⁸; and
the hydrocarbon groups represented by R¹¹ are each independently optionally substituted with —OC(O)O—R⁵³, —C(O)O—R⁵⁴, or —OC(O)—R⁵⁵.

43. (canceled)

44. A composition comprising a plurality of lipid nanoparticles, wherein the lipid nanoparticles comprise an agent, an ionizable lipid, and at least one of EDPPC and DPTAP, wherein the ionizable lipid is a lipid according to formula I or II or a pharmaceutically acceptable salt thereof: 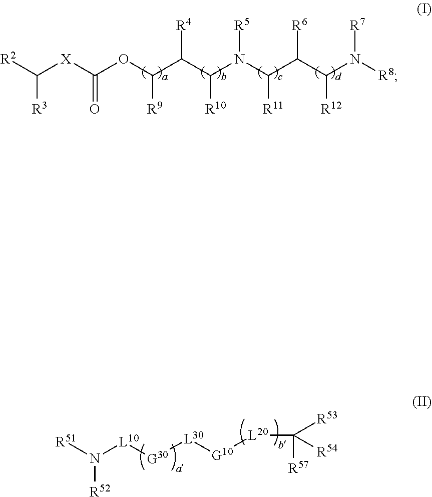
wherein
X is —NR¹— or —O—;
R¹ is a hydrogen atom, a hydrocarbon group having 6 to 24 atoms, or R²¹-L¹-R²²—;
R²¹ is a hydrocarbon group having 1 to 24 carbon atoms;
L¹ is —O(CO)O—, —O(CO)—, —(CO)O—, —O—, or 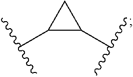 [SMILES: *C1CC1*]
R²² is a divalent hydrocarbon linking group having 1 to 18 carbon atoms;
R² and R³ are each independently a hydrogen atom, a hydrocarbon group having 3 to 24 carbon atoms, or R³¹-L¹-R³²—;
R³¹ is a hydrocarbon group having 1 to 24 carbon atoms;
L² is —O(CO)O—, —O(CO)—, —(CO)O—, —O—, or 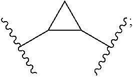 [SMILES: *C1CC1*]
R³² is a divalent hydrocarbon linking group having 1 to 18 carbon atoms;
R⁴, R⁵, R⁶, R⁷, R⁸, R⁹, R¹⁰, R¹¹, and R¹² are each independently a hydrogen atom or an alkyl group having 1 to 18 carbon atoms optionally substituted with R³³, where one or more pairs of groups selected from R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸ may be linked to each other to form a 4- to 7-membered ring of carbon and nitrogen atoms which may contain an oxygen atom;
R³³ is a hydroxyl group, a carboxyl group, —NR⁴⁵R⁴⁶, —O(CO)O—R⁴¹, —O(CO)—R⁴², —(CO)O—R⁴³, —O—R⁴⁴, or an aryl or heteroaryl group optionally substituted with R³⁴;
R³⁴ is an alkyl group having 1 to 18 carbon atoms, a hydroxyl group, a carboxyl group, —NR⁴⁵R⁴⁶, —O(CO)O—R⁴¹, —O(CO)—R⁴², —(CO)O—R⁴³, or —O—R⁴⁴;
R⁴¹, R⁴², R⁴³, R⁴⁴, R⁴⁵, and R⁴⁶ are each independently a hydrocarbon group having 1 to 18 carbon atoms;
a, b, c, and d are each independently an integer from 0 to 3, where a+b is 1 or more and c+d is 1 or more;
R⁵¹ and R⁵² are each independently an alkyl group having 1 to 18 carbon atoms optionally substituted with R³⁵,
R³⁵ is a hydroxyl group, -G²⁰-CH(R⁵⁵)(R⁵⁶), —N(R⁵⁸)(R⁵⁹), or -G²⁰-R⁶⁰;
G²⁰ is —(CO)O— or —O(CO)—;
R⁵⁵ and R⁵⁶ are each independently hydrogen or an alkyl group having 1 to 18 carbon atoms;
R⁵⁸ and R⁵⁹ are each independently hydrogen or a cycloalkyl group having 3 to 6 carbon atoms, optionally substituted with R³⁶;
R⁶⁰ is alkyl group having 1 to 18 carbon atoms or a cycloalkyl group having 3 to 6 carbon atoms;
R³⁶ is —N(R⁶¹)(R⁶²), or -G²⁰-R⁶⁵;
R⁶¹ and R⁶² are each independently hydrogen or a cycloalkyl group having 3 to 6 carbon atoms;
R⁶⁵ is an alkyl group having 1 to 18 carbon atoms, -L⁴⁰-CH(R⁶⁶)(R⁶⁷), or -G²⁰-R⁶⁶;
L⁴⁰ is a divalent alkyl group containing 1 to 6 carbon atoms;
R⁶⁶ and R⁶⁷ are each independently an alkyl group containing 1 to 10 carbon atoms or an alkoxy group containing 1 to 10 carbon atoms;
L¹⁰ is a divalent alkyl group containing 1 to 10 carbon atoms;
G³⁰ is —S(CO)N(R⁶⁴)—;
R⁶⁴ is -L³⁰-G²⁰-CH(R⁵⁵)(R⁵⁶);
a′ is 0 or 1;
G¹⁰ is G²⁰, —O(CO)O—, or —N(R⁶³)C(O)—;
R⁶³ is an alkyl group containing 1 to 18 carbon atoms;
L²⁰ is a divalent alkyl group containing 1 to 6 carbon atoms;
b′ is 0 or 1;
R⁵³, R⁵⁴, and R⁵⁷ are each independently hydrogen or an alkyl group containing 1 to 18 carbon atoms optionally substituted with R³⁶; and
L³⁰ is a single bond or an alkyl group containing 1 to 18 carbon atoms.

45. The composition of claim 1, wherein the agent is a bioactive agent.

46-49. (canceled)

50. The composition of claim 1, wherein the agent is a nucleic acid.

51-53. (canceled)

54. A method of delivering an agent to one or more of the brain, heart, muscles, and kidneys of a subject in need thereof, comprising systemically administering to the subject a composition comprising a plurality of lipid nanoparticles, wherein the lipid nanoparticles comprise the agent, an ionizable lipid, and a fully saturated cationic lipid.

55-68. (canceled)

69. A method of delivering an agent to a subject in need thereof, comprising administering to the subject a composition of claim 1.

70. A method of treating a subject having a disease, disorder or condition beneficially treated by an agent, comprising administering to the subject a therapeutically effective amount of a composition of claim 1.


SEQUENCE LISTING (2 sequences)

SEQ ID NO: 1 - RNA, 21 nt, synthetic construct
  source 1..21: mol_type = other RNA
  modified_base 1..2: mod_base = cm
  modified_base 13: mod_base = cm
  modified_base 14: mod_base = um
  modified_base 18..19: mod_base = um
  modified_base 20: mod_base = OTHER; note = thymine
  modified_base 21: mod_base = OTHER; note = thymine
nnaaaagagaganngganntt

SEQ ID NO: 2 - RNA, 21 nt, synthetic construct
  source 1..21: mol_type = other RNA
  modified_base 5: mod_base = cm
  modified_base 20: mod_base = OTHER; note = thymine
  modified_base 21: mod_base = OTHER; note = thymine
aatcnagtctctcttttggtt